(19) United States
(12) Patent Application Publication
(10) Pub. No.: US 2018/0127770 A1
(43) Pub. Date: May 10, 2018

(54) XA1-MEDIATED RESISTANCE TO TALE-CONTAINING BACTERIA

(71) Applicant: Iowa State University Research Foundation, Inc., Ames, IA (US)

(72) Inventors: Bing Yang, Ames, IA (US); Bo Liu, Ames, IA (US); Gong-you Chen, Ames, IA (US); Zhiyuan Ji, Ames, IA (US)

(21) Appl. No.: 15/645,072

(22) Filed: Jul. 10, 2017

Related U.S. Application Data
(60) Provisional application No. 62/359,824, filed on Jul. 8, 2016.

(51) Int. Cl. C12N 15/82; C07K 14/415

(52) U.S. Cl. CPC C12N 15/8281; C07K 14/415

(57) ABSTRACT

The present invention generally provides methods to generate broad-spectrum resistance to Xanthomonas pathogenic bacteria in plants. The invention relates to nucleic acid sequences identified, which are associated with broad spectrum disease resistance including Xa1, an NBS-LRR (Nucleotide Binding Site-Leucine-Rich Repeats) type R gene in rice (SEQ ID NOS: 1 and 2) and Xa1 homolog gene, Xa2, (SEQ ID NOS: 3 and 4) and homologs thereof. Further, novel iTALE (interfering transcription activator-like effectors), have also been identified, for example, iTAL3a (SEQ ID NOS: 5 and 6) and iTAL3b (SEQ ID NOS: 7 and 8) and homologs thereof. Modulation of these proteins can improve disease resistance in plants.

### CROSS-REFERENCE TO RELATED APPLICATIONS

This application claims priority under 35 U.S.C. § 119 to provisional application Ser. No. 62/359,824, filed Jul. 8, 2016, herein incorporated by reference in its entirety.

### GRANT REFERENCE

This invention was made with government support under the US National Science Foundation research grants IOS-1238189 and IOS-1258103. The government has certain rights in the invention.

### FIELD OF THE INVENTION

The present invention relates generally to plant molecular biology and genetic approaches for engineering enhanced and broad-spectrum resistance to bacterial diseases in plants. Disclosed herein are methods of producing genetically manipulated plants with increased disease resistance, particularly integration of exogenous sequences and/or modified target gene sequences, which confer disease resistance to Xanthomonas pathogenic bacteria but retain normal plant development, polynucleotides for engineering the same, and genetically manipulated plants and seeds generated therefrom.

### BACKGROUND OF THE INVENTION

Plant diseases are largely a consequence of molecular interactions between pathogens and their host plants. Significant yield loss in crop production can result when molecular battles are won by the pathogens. Bacterial pathogens depend in part on the type III secretion system to translocate effector proteins into host cells, where they exert a number of effects that help the pathogen to survive and to escape an immune response (2). In response, plants use diverse resistance (R) genes to recognize the cognate bacterial type III effectors in a gene-for-gene fashion, resulting in cultivar/race specific disease resistance that prevents a state of disease susceptibility in plants (3). Bacteria, in turn, diversify or inactivate the effector genes to evade the R gene recognition or evolve new effectors to suppress the resistance triggered by other distinct type III effectors (4, 5).

TALEs (transcription activator-like effectors) represent the largest type III effector family that are highly conserved at the nucleotide and amino acid levels (6), and are distinguishable by the varying number of central repeats of 34 amino acids and composition of the variable 12th and 13th amino acids of each repeat. TALEs also contain the characteristic nuclear localization motifs and transcription activation domain at their carboxyl termini (7). The repeat number and composition determine the specificity of each TAL effector for its DNA recognition in the promoter of host gene, a feature that has spawned the development of TALE-based biotechnologies including TALENs (TALE nucleases) for genome editing (8).

TALEs play an important role in the pathogenesis of some Xanthomonas bacteria (7), including X. oryzae pv. oryzae (Xoo) and X. oryzae pv. oryzicola (Xoc), the causal agents of bacterial blight and leaf streak, respectively, in rice (9, 10). Similar proteins can be found in the pathogenic bacterium Ralstonia solanacearum and Burkholderia rhizoxinica, as well as yet unidentified marine microorganisms. Bacterial TALEs target host genes of susceptibility (S gene) in a sequence specific manner, resulting in enhanced bacterial growth and development of disease symptom (11). To counteract such virulence strategy, host plants diversify the TALE binding elements in the promoters of S genes, resulting in recessive R genes (12). In addition, plants have also evolved so-called “executor” R genes to lure TAL effectors into triggering resistance in a way that the pathogens direct expression of S genes (13). Finally, unlike plants that involve transcription activation of R or S genes by TALEs, in one case, tomato uses the NBS-LRR type R gene Bs4 to activate resistance corresponding to AvrBs4, a TALE whose transcriptional functionality is not required for Bs4 resistance activation as severely truncated AvrBs4 derivatives also trigger resistance (14).

There remains a need to identify novel molecular factors which contribute to plant pathogenesis and resistance. Extensive efforts have been made to identify protein-coding genes of non-coding RNAs involved in host/microbe interactions but the possible role of pseudogenes in these interactions have yet to be elucidated (15, 16). Pseudogenes are genomic loci that resemble known functional gens but possess deletions/insertions, premature stop codon and frameshift mutations, resulting in genes non-transcribed, noncoding RNAs and truncated proteins if transcribed (17, 18).

Furthermore, there is a need to develop methods of generating disease resistance in plants, and in particular, to develop methods of generating broad-spectrum resistance to bacterial blight, enhanced resistance to bacterial leaf streak, and resistance to related diseases caused by TALE secreting pathogens.

### SUMMARY OF THE INVENTION

The present invention generally provides methods to generate broad-spectrum resistance to Xanthomonas pathogenic bacteria, in particular, resistance to bacterial blight disease, and enhanced resistance to bacterial leaf streak in plants. Thus, in one aspect, this invention relates to nucleic acid sequences identified, which are associated with broad spectrum disease resistance including Xa1, an NBS-LRR (Nucleotide Binding Site-Leucine-Rich Repeats) type R gene in rice (SEQ ID NOS: 1 and 2) and Xa1 homolog gene, Xa2, (SEQ ID NOS: 3 and 4) and homologs thereof. Further, novel iTALE (interfering transcription activator-like effectors), have also been identified, for example, iTAL3a (SEQ ID NOS: 5 and 6) and iTAL3b (SEQ ID NOS: 7 and 8) and homologs thereof.

In another aspect of the present invention, expression cassettes and transformation vectors comprising the identified and isolated nucleotide sequences are disclosed. The transformation vectors can be used to transform plants to modulate Xanthomonas resistance genes in transformed cells. Transformed cells as well as regenerated transgenic plants and seeds containing and expressing the isolated and identified DNA sequences and protein products are also provided.

Therefore, in one aspect, the present invention relates to an isolated and identified nucleic acid comprising an isolated polynucleotide sequence encoding a Xanthomonas resistance gene product that confers improved bacterial blight disease and/or bacterial leaf streak resistance. The methods of the invention are practiced with an isolated or recombinant polynucleotide comprising a member selected from the group consisting of: (a) a polynucleotide, or a complement thereof, comprising, e.g., at least about 70%, at least about 75%, at least about 80%, at least about 85%, at least about 90%, at least about 95%, at least about 99%, about 99.5% or more sequence identity to SEQ ID NO: 1, 3, 5, or 7, or a subsequence thereof, or a conservative variation thereof, (b) a polynucleotide, or a complement thereof, encoding a polypeptide sequence of SEQ ID NO: 2, 4, 6, or 8, or a subsequence thereof, or a conservative variation thereof, (c) a polynucleotide, or a complement thereof, that hybridizes under stringent conditions over substantially the entire length of a polynucleotide subsequence comprising at least 100 contiguous nucleotides of SEQ ID NO: 1, 3, 5 or 7, or that hybridizes to a polynucleotide sequence of (a) or (b); and, (d) a polynucleotide that is at least about 85% identical to a polynucleotide sequence of (a), (b) or (c). In at least some embodiments the polynucleotide includes at least one base change so as not to be the genomic sequence. In certain embodiments, the polynucleotide or polypeptide includes one or more base changes to that the sequence is not the naturally occurring sequence.

Furthermore, it is within the scope of the present invention to inhibit or provide antagonists of the novel iTALE (interfering transcription activator-like effectors), for example, iTAL3a (SEQ ID NOS: 5 and 6) and iTAL3b (SEQ ID NOS: 7 and 8) and homologs thereof. Accordingly, nucleotide sequences, polypeptide sequences and fragments thereof are provided which enhance resistance to Xanthomonas and other TALE secreting pathogens. The sequences reported herein are non-limiting examples of potential coding sequences of these genes.

In another aspect, the present invention relates to a recombinant expression cassette comprising a nucleic acid as described, supra. Additionally, the present invention relates to a vector containing the recombinant expression cassette. Further, the vector containing the recombinant expression cassette can facilitate the transcription and translation of the nucleic acid in a host cell. The present invention also relates to host cells able to express the polynucleotide of the present invention. A number of host cells could be used, such as but not limited to, microbial, mammalian, plant, or insect. Thus the invention is also directed to transgenic cells, containing the nucleic acids of the present invention as well as cells, plants, tissue cultures and ultimately lines derived therefrom.

This invention also provides an isolated polypeptide comprising (a) a polypeptide comprising at least 90% or 95% sequence identity to SEQ ID NO: 2, 4, 6, or 8 or fragment thereof (b) a polypeptide encoded by a nucleic acid of the present invention or fragment thereof; and (c) a polypeptide comprising a Xanthomonas resistance activity and comprising conserved structural domain motifs of the same.

Another aspect of the invention provides genetically manipulated disease resistant plants and seed of said plants. Another aspect of the invention comprises progeny plants, or seeds, or regenerable parts of plants and seeds of the genetically manipulated plants.

Another aspect of the invention, disclosed herein are methods of breeding plants of the invention comprising crossing a plant of the invention with a second plant to yield a progeny with enhanced resistance to Xanthomonas pathogenic bacteria, wherein at least partial disease resistance is introgressed from the plant of the invention into the second plant.

The plants in accordance with the present invention, in a non-limiting example, may be rice, tomato, citrus, wheat, cotton, pepper, beans, cucumber, cabbage, barley, oats and corn.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### DESCRIPTION OF THE FIGURES

FIG. 1. TALE (transcription activator-like effectors) gene cluster deletions in PXO99. (A) All 19 TALE genes of PXO99 are present in 9 clusters (cluster 8 is a duplication of cluster 7), and each cluster consists of one to five members. Name of constructs for deletion, order of sequential deletions, known TALE genes and their targets (resistance or susceptible genes in host rice) are indicated above or below the arrow for each TALE gene. (B) Southern blotting analysis the TALE gene cluster mutants. After 8 rounds of sequential deletions, eight TALE mutants (PA to PH) including the TALE-free mutant PH were obtained. DNA ladder was denoted at the rightside and TALE genes corresponding to the bands were marked at the left side.

FIG. 2: iTALE genes Tal3a and Tal3b interfere with Xa1-resistance. a, Gene structures of Tal3a and Tal3b relative to pthXo1. Number of nucleotides (nt) is denoted above each domain. Straight gray Lines represent nt deletions in Tal3a and Tal3b relative topthXo1. b, Disease reactions of IRBB1 and IR24. Strains are indicated above the leaves. Brown coloration indicates HR, and clearing spots indicate disease reaction. c, Lesion lengths in IR24 and IRBB1 caused by Xoo strains as measured at 12 day post inoculation (DPI). d, Lesion lengths in four independent Xa1 transgenic lines at 12 DPI. e, Xa1 transgenic line #17 showed HR (brown coloration) to ΔTal3 but disease reactions to ΔTal3 complementing strains and PXO99. Photos were taken at 3 DPI. Error bars indicate SD

FIG. 3. Sequence information of Tal3a and Tal3b. (A) The central repeats of predicted Tal3a and Tal3b in order of RVD (repeat variable di-amino acids). Underlined RVDs represent a repeat with a unique length of 28 amino acids compared to normal 34 amino acids. An asterisk (*) denotes the last half repeat. (B) Partial sequences of Tal3a and the predicted truncated effector relative topthXo1 (SEQ ID NOs: 9-11). (C) Partial sequences of Tal3b and the predicted truncated effector relative topthXo1 (SEQ ID NOs: 12-17). (D) Predicted amino acid sequences of the C-termini of Tal3a and Tal3b compared to PthXo1 (SEQ ID NOs: 18-20). Underlined sequences are identical for three TALEs; letters in red are predicted nuclear localization signals; bold letters are C-terminal transcription activation domain in PthXo1 but truncated in Tal3a and Tal3b.

FIG. 4. iTALE genes Tal3a and Tal3b were expressed in bacteria. RNA from two PXO99 cultures (as indicated above each lane) was used for cDNA synthesis and PCR with gene specific primers. 16S rRNA gene expression was used as an internal control.

FIG. 5. Western blotting analysis of Tal3 and Tal3b. Bacterial extract was separated through gel electrophoresis and blotted/probed with the monoclonal antibody against the epitope FLAG.

FIG. 6. Western blotting analysis of Tal3a and its repeat deletions.

Bacterial extract was separated through gel electrophoresis and blotted/probed with monoclonal antibody against epitope FLAG.

FIG. 7. Unique domains in Tal3a are required for its suppressive activity. a, Lesion lengths in IRBB1 and IR24 caused by Xoo strains as indicated below each column at 14 DPI. Δ1-15, Δ1-10 and Δ1-2 represent Tal3a with the N-terminal 15, 10 and 2 repeats deleted, respectively. b, Lesion lengths in Kitaake (WT) and Xa1 transgenic plants as assessed similarly in (a) but at 12 DPI. c, Both Tal3a N-terminal and C-terminal structures are required for suppression of Xa1-mediated HR triggered by ΔTal3. Xo1N-Tal3aRC is a Tal3a variant containing PthXo1 N-terminus and Tal3a central repetitive and C-terminal domains. Tal3aNR-Xo1C is a hybrid of Tal3a N-terminal and repetitive domains and PthXo1 C-terminus. Photos were taken at 3 DPI. Error bars indicate SD.

FIG. 8. TAL effectors containing repeats of various TAL effector genes in Tal3a scaffold function as resistance suppressors. a, Disease reactions of IR24 and IRBB1 in response to various Xoo strains. Photos were taken 3 DPI. Clearing coloration indicates disease reaction and brown coloration denotes HR. b, Lesion lengths caused by different strains in IR24 and IRBB1. Error bars represent SD.

FIG. 9. N-terminal domain of Tal3a is required for its ability to suppress resistance to ΔTal3 in IRBB1. The Xoo strains were indicated below each column. avrXa7N-Tal3aRC is a chimeric gene of AvrXa7 N-terminus coding region and Tal3a central and C-terminal domain coding regions. Lesion lengths were measured at 14 DPI.

FIG. 10. Nuclear localization motifs in Tal3a and Tal3b are required for their suppressive activities. a, Amino acids at the NLS and replacements are in red and underlined (SEQ ID NOs: 21-26). b, NLS motifs in Tal3a and Tal3b are functional to direct the GFP-tagged effectors into nuclei of rice protoplasts. c, NLS motifs are required for Tal3a and Tal3b to interfere with the Tal3 triggered resistance in IRBB1. Lesion lengths of rice leaves caused by different strains as indicated below each column and measured at 12 DPI. Error bars indicate SD.

FIG. 11. HR to various full-length TALEs in Xa1-transgenic plants.

Leaves of Xa1 transgenic Kitaake were inoculated with PH strains containing individual TALE genes. PH is a PXO99 TALE-free mutant. pthXo1, Tal4 and Tal9d are three plasmid-borne TALE genes from PXO99. Tal3aFL and Tal3bFL are avrXa7 variants swapped with Tal3a and Tal3b repetitive domains, resulting in the restructured full-length versions of Tal3a and Tal3b, respectively. Tal3aNR-Xo1C consists of Tal3a N-terminal and repetitive domains and PthXo1 C-terminus. Xo1N-Tal3aRC is a Tal3a variant containing PthXo1 N-terminus and Tal3a central repetitive and C-terminal domains. Photos were taken at 3 DPI.

FIG. 12. Full-length C-terminus of TALE enabled Tal3a and Tal3b to trigger HR specifically in IRBB1. The Xoo strains were indicated above the leaves. PH is the TALE gene free of PXO99. Photos were taken at 3 DPI.

FIG. 13. Nuclear localization motifs in PthXo1 and AvrXa7 are required for activation of Xa1 resistance. a, Amino acids at the NLS and replacements in PthXo1 and its derivatives are in red and underlined (SEQ ID NOs: 27-33). Sequences for AvrXa7 were as described in a prior study (ref. 1). b, Disease reactions in Xa1 containing plants to various Xoo strains as indicated above the leaves. Photos were taken 3 DPI.

FIG. 14. Overexpressed Xa1 confers resistance to ΔTal3 and iTALE gene

Tal3a suppresses the resistance. Four lines of transgenic Kitaake expressing Xa1 under the rice ubiquitin promoter were resistant to ΔTal3 strain but susceptible to the complementing strain ΔTal3 with Tal3a. The non-transgenic plants (CK) were susceptible to both ΔTal3 and ΔTal3/Tal3a. Lesion lengths were measured 12 DPI. Bars indicate SD.

FIG. 15. Rice defense genes in IRBB1 are induced by TALE and the gene induction is suppressed by Tal3a. Three-weeks-old plants of IRBB1 were inoculated with water (wounding), PXO99, ΔTal3 and ΔTal3/Tal3a, and total RNA was isolated at 24 h after inoculation. cDNA prepared from the samples was subjected to quantitative RT-PCR using primers specific to Xa1 (a), peroxidase PXO22.3 (Os07g48020) (b), PBZ (probenazole-inducible gene, Os12g36880) (c) and PR1 (pathogenesis-related gene, Os07g03730) (d). Gene-specific primers for the rice actin gene (Os03g50885) were used for control. The expression level relative to that of non-inoculated leaves is presented in average threshold cycle (Ct) using the 2-ΔΔCt method (ref. 2).

FIG. 16. Unrooted phylogenic tree of 18 iTALE genes from Xoo (n=4) and Xoc (n=14). Bootstrap values shown at nodes were obtained from 1,000 trials, and branch lengths correspond to the divergence of DNA sequences, as indicated by the relative scale. Neighbor-Joining method (Software Mega 6, http://www.megasoftware.net) was used for the tree.

FIG. 17. Alignment of repeats of 18 iTALEs as represented by 12th and 13th amino acids (RVD) of each repeat. The single amino acid code is used for each amino residue. An asterisk (*) denotes the missing 13th amino acid. Underlined RVD represents a repeat with a deletion of 6 amino acids (23th to 28th). (A) and (B) represent type A and type B effectors, respectively (SEQ ID NOs: 19, 20).

FIG. 18. iTALEs enable IRBB1-incompatible strain T7174 compatible to IRBB1. Xoo strains with the plasmid-borne Tal3a and Tal3b from PXO99 and Tal6a from T7174 in the scaffold of Tal3a were inoculated in IR24 (T7174 compatible) and IRBB1. Lesion lengths were measured 12 days post inoculation. Error bars are SD.

FIG. 19. A and B Suppressive activity of iTALE genes from Xoo and Xoc. a, iTALE genes from the Xoo PXO86 and Xoc RS105 and BXOR1 overcome Xa1 resistance triggered by ΔTal3. b, Inactivation of iTALE gene Tal5e enables the mutant (ΔTal5e) to trigger resistance in IRBB1. c, iTALE genes from Xoo PXO86 and PXO99 restore the Xoc mutant (ΔTal5e) ability to cause disease in IRBB1. Photos were taken at 4 DPI.

FIG. 20. iTALEs from Xoo and Xoc suppress ΔTal3 triggered resistance in IRBB1. Five-week old IR24 and IRBB1 plants were inoculated with strains as indicated below each column using leaf-tip clipping method. Lesion lengths were measured at 13 DPI.

Error bars indicate SD.

FIG. 21. Suppressive activity of the Xoo iTALE genes in Xoc. iTALE genes from PXO86 and PXO99 of Xoo restore the Xoc mutant (ΔTal5e) ability to cause disease in IRBB1 in term of lesion length measured at 7 DPI. Error bars represent SD.

FIGS. 22A and B. Transgenic wheat (T1 plants) containing rice disease resistance gene Xa1 confers resistance to wheat bacterial blight, caused by Xanthomonas translucens a. Gene construct of Xa1 for wheat transformation. b. Transgenic seedlings (20-days old) were infiltrated with bacterial inoculum and photographed 4 days after inoculation. Please note the water soaking spots were confined at the inoculation spots in transgenic plants while in wild type plant water soaking spread far beyond the inoculation spots.

### DETAILED DESCRIPTION OF THE INVENTION

The present invention now will be described more fully with reference to the accompanying examples. The invention may be embodied in many different forms and should not be construed as limited to the embodiments set forth in this application; rather, these embodiments are provided so that this disclosure will satisfy applicable legal requirements.

Many modifications and other embodiments of the invention will come to mind to one skilled in the art to which this invention pertains, having the benefit of the teachings presented in the descriptions and the drawings herein. As a result, it is to be understood that the invention is not to be limited to the specific embodiments disclosed and that modifications and other embodiments are intended to be included within the scope of the appended claims. Although specific terms are used in the specification, they are used in a generic and descriptive sense only and not for purposes of limitation.

### General

In order to provide a clear and consistent understanding of the specification and the claims, including the scope given to such terms, the following definitions are provided. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, e.g., Sambrook et al. MOLECULAR CLONING: A LABORATORY MANUAL, 2d ed., Cold Spring Harbor Laboratory Press, 1989; 3d ed., 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

Unless otherwise specified, “a,” “an,” “the,” and “at least one” are used interchangeably and mean one or more than one.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific or conformation specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule.

A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids and includes hypervariable diresidues at positions 12 and/or 13 referred to as the Repeat Variable Diresidue (RVD) involved in DNA-binding specificity. TALE repeats exhibit at least some sequence homology with other TALE repeat sequences within a naturally occurring TALE protein. See, e.g., U.S. Pat. No. 8,586,526.

Zinc finger binding and TALE domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. No. 8,586,526, U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970WO 01/88197 and WO 02/099084.

As used herein, “vector” includes reference to a nucleic acid used in transfection of a host cell and into which can be inserted a polynucleotide. Vectors are often replicons. Expression vectors permit transcription of a nucleic acid inserted therein.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

A “homologous”, “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. GenBank® is the recognized United States-NIH genetic sequence database, comprising an annotated collection of publicly available DNA sequences, and which further incorporates submissions from the European Molecular Biology Laboratory (EMBL) and the DNA DataBank of Japan (DDBJ), see Nucleic Acids Research, January 2013, v 41(D1) D36-42 for discussion. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, functional activity, protein binding or nucleotide binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   See also, Creighton (1984) Proteins W.H. Freeman and Company.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, that uses a “donor” molecule to template repair of a “target” molecule (i.e., the one that experienced the double-strand break), and is variously known as “non-crossover gene conversion” or “short gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possess catalytic activity for DNA cleavage. A cleavage domain can be contained in a single polypeptide chain or cleavage activity can result from the association of two (or more) polypeptides.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528, 2008/0131962 and 2011/0201055, incorporated herein by reference in their entireties.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes. “Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of H1 is generally associated with the linker DNA. For purposes of the present disclosure, the term “chromatin” is meant to encompass all types of cellular nucleoprotein, both prokaryotic and eukaryotic. Cellular chromatin includes both chromosomal and episomal chromatin.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the EcoRI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present in cells only during the early stages of development of a flower is an exogenous molecule with respect to the cells of a fully developed flower. Similarly, a molecule induced by heat shock is an exogenous molecule with respect to a non-heat-shocked cell. An exogenous molecule can comprise, for example, a coding sequence for any polypeptide or fragment thereof, a functioning version of a malfunctioning endogenous molecule or a malfunctioning version of a normally-functioning endogenous molecule. Additionally, an exogenous molecule can comprise a coding sequence from another species that is an ortholog of an endogenous gene in the host cell.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases. Thus, the term includes “transgenes” or “genes of interest” which are exogenous sequences introduced into a plant cell.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, protoplast transformation, silicon carbide (e.g., WHISKERS™), Agrobacterium-mediated transformation, lipid-mediated transfer (i.e., liposomes, including neutral and cationic lipids), electroporation, direct injection, cell fusion, particle bombardment (e.g., using a “gene gun”), calcium phosphate co-precipitation, DEAE-dextran-mediated transfer and viral vector-mediated transfer.

By contrast, an “endogenous” molecule is one that is normally present in a particular cell at a particular develop-mental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein, the term “product of an exogenous nucleic acid” includes both polynucleotide and polypeptide products, for example, transcription products (polynucleotides such as RNA) and translation products (polypeptides).

A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. Transgenic progeny having the same nucleus with either heterozygous or homozygous chromosomes for the recombinant DNA are said to represent the same transgenic event. Once a transgene for a trait has been introduced into a plant, that gene can be introduced into any plant sexually compatible with the first plant by crossing, without the need for directly transforming the second plant. The heterologous DNA and flanking genomic sequence adjacent to the inserted DNA will be transferred to progeny when the event is used in a breeding program and the enhanced trait resulting from incorporation of the heterologous DNA into the plant genome will be maintained in progeny that receive the heterologous DNA.

The term “event” also refers to the presence of DNA from the original transformant, comprising the inserted DNA and flanking genomic sequence immediately adjacent to the inserted DNA, in a progeny that receives inserted DNA including the transgene of interest as the result of a sexual cross of one parental line that includes the inserted DNA (e.g., the original transformant and progeny resulting from selfing) and a parental line that does not contain the inserted DNA. The term “progeny” denotes the offspring of any generation of a parent plant prepared in accordance with the present invention. A transgenic “event” may thus be of any generation. The term “event” refers to the original transformant and progeny of the transformant that include the heterologous DNA. The term “event” also refers to progeny produced by a sexual outcross between the transformant and another variety that include the heterologous DNA. Even after repeated back crossing to a recurrent parent, the inserted DNA and flanking DNA from the transformed parent is present in the progeny of the cross at the same chromosomal location.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene or gene product. Modulation of expression can include, but is not limited to, gene activation and gene repression and/or activation or repression of the gene product.

As used herein, the terms “resistance protein”, and “resistance gene” shall include any amino acid sequence or nucleotide sequence, respectively, which retain one or more of the properties of proteins listed herein in general. Such proteins may include Xa1 (SEQ ID NOS: 1 and 2), Xa2, (SEQ ID NOS: 3 and 4), iTAL3a (SEQ ID NOS: 5 and 6), iTAL3b (SEQ ID NOS: 7 and 8) and any conservatively modified variants, fragments, and homologs or full length sequences incorporating the same which retain the related resistance activity described herein. Resistance proteins are capable of suppressing, controlling, and/or preventing invasion by the pathogenic organism. A resistance protein will reduce the disease symptoms (i.e., leaf blight and/or leaf streak) resulting from pathogen challenge in a previously susceptible plant by at least about 2%, including but not limited to, about 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater. In particular embodiments, the disease symptoms resulting from pathogen challenge are reduced by resistance protein by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens. Resistance may vary from a slight increase in tolerance to the effects of the pathogen (e.g., partial inhibition) to total resistance such that the plant is unaffected by the presence of the pathogen. An increased level of resistance against a particular pathogen or against a wider spectrum of pathogens may both constitute complete resistance or improved resistance.

“Pathogen resistance”, “disease resistance” or “Xanthomanas resistance” is intended to mean that the plant avoids the disease symptoms that are the outcome of plant-pathogen interactions. That is, pathogens are prevented from causing plant diseases and the associated disease symptoms, or alternatively, the disease symptoms caused by the pathogen are minimized or lessened, such as, for example, the reduction of stress and associated yield loss.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing a resistance polypeptide shows a decrease in tissue necrosis (i.e., lesion diameter) or a decrease in plant death following pathogen challenge when compared to a control plant that was not engineered to express the resistance protein.

Alternatively, antipathogenic activity can be measured by a decrease in pathogen biomass. For example, a plant expressing a pathogen resistance protein is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference. According to the invention, the term “increased resistance” (against Xanthomonas.) is understood to mean that the genetically manipulated plants, or plant cells, according to the invention are less vigorously, and/or less frequently, affected by Xanthomonas than non-transformed wild type plants, or plant cells, which were otherwise treated in the same way (such as climate and cultivation conditions, pathogen type, etc.). According to the invention, the term “wild type” is to be understood as the respective non-genetically modified parent organism. The penetration efficiency as well as the rate of papillae formation offer a possibility to quantify the reaction of the plant to the pathogen infestation (see examples). The term “increased resistance” also comprises what is known as transient pathogen resistance, i.e. the transgenic plants, or plant cells, according to the invention have an increased pathogen resistance as compared to the respective wild type only for a limited period of time.

As used herein, “gene editing,” “gene edited” “genetically edited” and “gene editing effectors” refer to the use of naturally occurring or artificially engineered nucleases, also referred to as “molecular scissors.” The nucleases create specific double-stranded break (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganuclease re-engineered as homing endonucleases. The terms also include the use of transgenic procedures and techniques, including, for example, where the change is relatively small and/or does not introduce DNA from a foreign species. The terms “genetic manipulation” and “genetically manipulated” include gene editing techniques, as well as and/or in addition to other techniques and processes that alter or modify the nucleotide sequence of a gene or gene, or modify or alter the expression of a gene or genes.

As used herein “homing DNA technology” or “homing technology” covers any mechanisms that allow a specified molecule to be targeted to a specified DNA sequence including Zinc Finger (ZF) proteins, Transcription Activator-Like Effectors (TALEs) meganucleases, and the CRISPR/Cas9 system.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

As used herein, the term “plant” can include reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. Particularly preferred plants include rice, tomato, citrus, wheat, cotton, pepper, beans, cucumber, cabbage, barley, oats and corn.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. For instance, a coding region present in a wild-type microbe and encoding a Cas9 polypeptide is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide disclosed herein through the use of standard genetic and/or recombinant methodologies well known to one skilled in the art. A polynucleotide disclosed herein may be included in a suitable vector.

As used herein, “genetically modified” with reference to a cell, callus, tissue, plant, or animal which has been altered “by the hand of man.” A genetically modified cell, callus, tissue, plant, or animal has had an exogenous polynucleotide introduced thereto and includes progeny cells derived therefrom. Genetically modified, also refers to a cell callus, tissue, plant or animal that has been genetically manipulated such that endogenous nucleotides have been altered to include a mutation, such as a deletion, an insertion, a transition, a transversion, or a combination thereof, such as by gene editing. For instance, an endogenous coding region could be deleted. Such mutations may result in a polypeptide having a different amino acid sequence than was encoded by the endogenous polynucleotide. Another example of a genetically modified cell, callus, tissue, plant, or animal is one having an altered regulatory sequence, such as a promoter, to result in increased or decreased expression of an operably linked endogenous coding region.

It is also to be understood that two different transgenic and/or genetically manipulated plants can be mated to produce offspring that contain two independently segregating added, exogenous genes. Selecting of appropriate progeny can produce plants that are homozygous for both added, exogenous and/or modified genes. Alternatively, inbred lines containing the individual exogenous genes may be crossed to produce hybrid seed that is heterozygous for each gene, and useful for production of hybrid plants that exhibit multiple beneficial phenotypes as the result of expression of each of the exogenous genes. Descriptions of breeding methods that are commonly used for different traits and crops can be found in various references, e.g., Allard, “Principles of Plant Breeding,” John Wiley & Sons, NY, U. of CA, Davis, Calif., 50-98, 1960; Simmonds, “Principles of Crop Improvement,” Longman, Inc., NY, 369-399, 1979; Sneep and Hendriksen, “Plant Breeding Perspectives,” Wageningen (ed), Center for Agricultural Publishing and Documentation, 1979.

### Nucleic Acids

The present invention provides, inter alia, isolated nucleic acids of RNA, DNA, homologs, paralogs and orthologs and/or chimeras thereof, comprising an Xanthomonas resistance polynucleotide or protein encoded thereby. This includes naturally occurring as well as synthetic variants and homologs of the sequences.

Sequences homologous, i.e., that share significant sequence identity or similarity, to those provided herein derived from maize, rice or from other plants of choice, are also an aspect of the invention. Homologous sequences can be derived from any plant including monocots and dicots and in particular agriculturally important plant species, including but not limited to, crops such as soybean, wheat, corn (maize), potato, cotton, rape, oilseed rape (including canola), sunflower, alfalfa, clover, sugarcane, and turf, or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassava, turnip, radish, yam, and sweet potato; and beans. The homologous sequences may also be derived from woody species, such pine, poplar and eucalyptus, or mint or other labiates. In addition, homologous sequences may be derived from plants that are evolutionarily-related to crop plants, but which may not have yet been used as crop plants. Examples include deadly nightshade (Atropa belladona), related to tomato; jimson weed (Datura strommium), related to peyote; and teosinte (Zea species), related to corn (maize).

### Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may result in two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360).

For example, a clade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) Plant J. 16: 433-442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001), in Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence).

### Variant Nucleotide Sequences in the Non-Coding Regions

The Xanthomonas resistance nucleotide sequences are used to generate variant nucleotide sequences having the nucleotide sequence of the 5′-untranslated region, 3′-untranslated region, or promoter region that is approximately 70%, 75%, and 80%, 85%, 90% and 95% identical to the original nucleotide sequence. These variants are then associated with natural variation in the germplasm for component traits related to pathogen resistance. The associated variants are used as marker haplotypes to select for the desirable traits.

### Variant Amino Acid Sequences of Polypeptides

Variant amino acid sequences of the Xanthomonas resistance polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined herein is followed. Variants having about 70%, 75%, 80%, 85%, 90% and 95% nucleic acid sequence identity are generated using this method. These variants are then associated with natural variation in the germplasm for component traits related to pathogen resistance. The associated variants are used as marker haplotypes to select for the desirable traits.

The present invention also includes polynucleotides optimized for expression in different organisms. For example, for expression of the polynucleotide in a particular plant, the sequence can be altered to account for specific codon.

The Xanthomonas resistance nucleic acids which may be used for the present invention comprise isolated Xanthomonas resistance polynucleotides which are inclusive of:

(a) a polynucleotide encoding an Xanthomonas resistance polypeptide and conservatively modified and polymorphic variants thereof;
(b) a polynucleotide having at least 70% sequence identity with polynucleotides of (a) or (b);
(c) Complementary sequences of polynucleotides of (a) or (b).

In certain embodiments the nucleic acids includes at least one base substitution so that they do not recite naturally occurring nucleic acid sequences.

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRT□GAL, pNEO□GAL, pRS403, pRS404, pRS405, pRS406, pRS413, pRS414, pRS415, pRS416, lambda MOSSlox, and lambda MOSElox. Optional vectors for the present invention, include but are not limited to, lambda ZAP II, and pGEX. For a description of various nucleic acids see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90 9; the phosphodiester method of Brown, et al., (1979) Meth. Enzymol. 68:109 51; the diethylphosphoramidite method of Beaucage, et al., (1981) Tetra. Letts. 22(20): 1859 62; the solid phase phosphoramidite triester method described by Beaucage, et al., supra, e.g., using an automated synthesizer, e.g., as described in Needham VanDevanter, et al., (1984) Nucleic Acids Res. 12:6159 68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G> 7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in rice. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395); or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Sequence Shuffling

The present invention provides methods for sequence shuffling using polynucleotides of the present invention, and compositions resulting therefrom. Sequence shuffling is described in PCT publication No. 96/19256. See also, Zhang, et al., (1997) Proc. Natl. Acad. Sci. USA 94:4504-9; and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be an altered Km and/or Kcat over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

### Promoters, Terminators, Introns

A plant promoter fragment can be employed which will direct expression of a polynucleotide of the present disclosure in essentially all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters may be “inducible” promoters. Environmental conditions that may affect transcription by inducible promoters include pathogen attack, anaerobic conditions or the presence of light. Examples of inducible promoters are the Adhl promoter, which is inducible by hypoxia or cold stress, the Hsp70 promoter, which is inducible by heat stress and the PPDK promoter, which is inducible by light. Diurnal promoters that are active at different times during the circadian rhythm are also known (US Patent Application Publication Number 2011/0167517, incorporated herein by reference). Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations. If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adhl-S intron 1, 2 and 6, the Bronze-1 intron are known in the art. See generally, The Maize Handbook, Chapter 116, Freeling and Walbot, eds., Springer, New York (1994).

### Signal Peptide Sequences

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PR1b (Lind, et al., (1992) Plant Mol. Biol. 18:47-53) or the barley alpha amylase (BAA) (Rahmatullah, et al., (1989) Plant Mol. Biol. 12:119) or signal peptides which target proteins to the plastids such as that of rapeseed enoyl-Acp reductase (Verwaert, et al., (1994) Plant Mol. Biol. 26:189-202) are useful in the disclosure.

### Markers

The vector comprising the sequences from a polynucleotide of the present disclosure will typically comprise a marker gene, which confers a selectable phenotype on plant cells. The selectable marker gene may encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance. Also useful are genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

Constructs described herein may comprise a polynucleotide of interest encoding a reporter or marker product. Examples of suitable reporter polynucleotides known in the art can be found in, for example, Jefferson, et al., (1991) in Plant Molecular Biology Manual, ed. Gelvin, et al., (Kluwer Academic Publishers), pp. 1-33; DeWet, et al. (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330. In certain embodiments, the polynucleotide of interest encodes a selectable reporter. These can include polynucleotides that confer antibiotic resistance or resistance to herbicides. Examples of suitable selectable marker polynucleotides include, but are not limited to, genes encoding resistance to chloramphenicol, methotrexate, hygromycin, streptomycin, spectinomycin, bleomycin, sulfonamide, bromoxynil, glyphosate and phosphinothricin.

In some embodiments, the expression cassettes disclosed herein comprise a polynucleotide of interest encoding scorable or screenable markers, where presence of the polynucleotide produces a measurable product. Examples include a .beta.-glucuronidase, or uidA gene (GUS), which encodes an enzyme for which various chromogenic substrates are known (for example, U.S. Pat. Nos. 5,268,463 and 5,599,670); chloramphenicol acetyl transferase and alkaline phosphatase. Other screenable markers include the anthocyanin/flavonoid polynucleotides including, for example, a R-locus polynucleotide, which encodes a product that regulates the production of anthocyanin pigments (red color) in plant tissues, the genes which control biosynthesis of flavonoid pigments, such as the maize C1 and C2, the B gene, the p1 gene and the bronze locus genes, among others. Further examples of suitable markers encoded by polynucleotides of interest include the cyan fluorescent protein (CYP) gene, the yellow fluorescent protein gene, a lux gene, which encodes a luciferase, the presence of which may be detected using, for example, X-ray film, scintillation counting, fluorescent spectrophotometry, low-light video cameras, photon counting cameras or multiwell luminometry, a green fluorescent protein (GFP) and DsRed2 (Clontechniques, 2001) where plant cells transformed with the marker gene are red in color, and thus visually selectable. Additional examples include a p-lactamase gene encoding an enzyme for which various chromogenic substrates are known (e.g., PADAC, a chromogenic cephalosporin), a xylE gene encoding a catechol dioxygenase that can convert chromogenic catechols, an .alpha.-amylase gene and a tyrosinase gene encoding an enzyme capable of oxidizing tyrosine to DOPA and dopaquinone, which in turn condenses to form the easily detectable compound melanin.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as .beta.-galactosidase and fluorescent proteins such as green fluorescent protein (GFP) (Su, et al., (2004) Biotechnol Bioeng 85:610-9 and Fetter, et al., (2004) Plant Cell 16:215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the compositions and methods disclosed herein.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present invention, one may express a protein of the present invention in a recombinantly engineered cell such as bacteria, yeast, insect, mammalian, or preferably plant cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation, and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

In additional embodiments, enhancer elements may be introduced which increase expression of the polynucleotides of the invention.

One of skill would recognize that modifications could be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Expression in Eukaryotes

A variety of eukaryotic expression systems such as yeast, insect cell lines, plant and mammalian cells, are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, et al., (1982) Methods in Yeast Genetics, Cold Spring Harbor Laboratory is a well-recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired. A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HAS tk promoter or pgk (phosphoglycerate kinase) promoter), an enhancer (Queen, et al., (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7th ed., 1992).

Appropriate vectors for expressing proteins of the present invention in insect cells are usually derived from the SF9 baculovirus. Suitable insect cell lines include mosquito larvae, silkworm, armyworm, moth, and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague et al., J. Virol. 45:773 81 (1983)). Additionally, gene sequences to control replication in the host cell may be incorporated into the vector such as those found in bovine papilloma virus type vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the Xanthomonas resistance gene placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert an Xanthomonas resistancepolynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment. Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e. monocot or dicot, targeted for gene modification. Suitable methods of transforming plant cells include microinjection (Crossway, et al., (1986) Biotechniques 4:320-334; and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (see, for example, Sanford, et al., U.S. Pat. No. 4,945,050; WO 91/10725; and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. O. L. Gamborg & G. C. Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839; and Gordon-Kamm, et al., (1990) Plant Cell 2:603-618 (maize); Hooydaas-Van Slogteren & Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255; and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra; and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993; and Simpson, et al., (1986) Plant Mol. Biol. 6:403-15 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present invention including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application No. 604 662 A1 discloses a method for transforming monocots using Agrobacterium. European Application No. 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731; and Christou, et al., (1987) Proc. Natl. Acad. Sci. USA 84:3962. Direct uptake of DNA into protoplasts using CaCl2 precipitation, polyvinyl alcohol, or poly-L-ornithine has also been reported. See, e.g., Hain, et al., (1985) Mol. Gen. Genet. 199:161; and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505; and Spencer, et al., (1994) Plant Mol. Biol. 24:51-61.

### Reducing the Activity of an Xanthomonas Resistance Polypeptide

In certain embodiments the invention may include modulation of the Xanthomonas resistance gene to reduce or eliminate the activity of an Xanthomonas resistance polypeptide, perhaps during certain developmental stages or tissues etc., by transforming a plant cell with an expression cassette that expresses a polynucleotide that inhibits the expression of the Xanthomonas resistance polypeptide. The polynucleotide may inhibit the expression of the Xanthomonas resistance polypeptide directly, by preventing transcription or translation of the Xanthomonas resistance messenger RNA, or indirectly, by encoding a polypeptide that inhibits the transcription or translation of an Xanthomonas resistance gene encoding an Xanthomonas resistance polypeptide. Methods for inhibiting or eliminating the expression of a gene in a plant are well known in the art, and any such method may be used in the present invention to inhibit the expression of the Xanthomonas resistance polypeptide. Many methods may be used to reduce or eliminate the activity of an Xanthomonas resistance polypeptide. In addition, more than one method may be used to reduce the activity of a single Xanthomonas resistance polypeptide.

### 1. Polynucleotide-Based Methods:

In some embodiments of the present invention, a plant is transformed with an expression cassette that is capable of expressing a polynucleotide that inhibits the expression of an Xanthomonas resistance polypeptide of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one Xanthomonas resistance polypeptide is an expression cassette capable of producing an RNA molecule that inhibits the transcription and/or translation of at least one Xanthomonas resistance polypeptide of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of an Xanthomonas resistance polypeptide are given below.

i. Sense Suppression/Cosuppression

In some embodiments of the invention, inhibition of the expression of an Xanthomonas resistance polypeptide may be obtained by sense suppression or cosuppression. For cosuppression, an expression cassette is designed to express an RNA molecule corresponding to all or part of a messenger RNA encoding an Xanthomonas resistance polypeptide in the “sense” orientation. Over expression of the RNA molecule can result in reduced expression of the native gene. Accordingly, multiple plant lines transformed with the cosuppression expression cassette are screened to identify those that show the greatest inhibition of Xanthomonas resistance polypeptide expression.

The polynucleotide used for cosuppression may correspond to all or part of the sequence encoding the Xanthomonas resistance polypeptide, all or part of the 5′ and/or 3′ untranslated region of an Xanthomonas resistance polypeptide transcript, or all or part of both the coding sequence and the untranslated regions of a transcript encoding an Xanthomonas resistance polypeptide. In some embodiments where the polynucleotide comprises all or part of the coding region for the Xanthomonas resistance polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763; and U.S. Pat. Nos. 5,034,323, 5,283,184, and 5,942,657; each of which is herein incorporated by reference. The efficiency of cosuppression may be increased by including a poly-dT region in the expression cassette at a position 3′ to the sense sequence and 5′ of the polyadenylation signal. See, U.S. Patent Publication No. 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323; herein incorporated by reference.

ii. Antisense Suppression

In some embodiments of the invention, inhibition of the expression of the Xanthomonas resistance polypeptide may be obtained by antisense suppression. For antisense suppression, the expression cassette is designed to express an RNA molecule complementary to all or part of a messenger RNA encoding the Xanthomonas resistance polypeptide. Over expression of the antisense RNA molecule can result in reduced expression of the native gene. Accordingly, multiple plant lines transformed with the antisense suppression expression cassette are screened to identify those that show the greatest inhibition Xanthomonas resistance polypeptide expression.

The polynucleotide for use in antisense suppression may correspond to all or part of the complement of the sequence encoding the Xanthomonas resistance polypeptide, all or part of the complement of the 5′ and/or 3′ untranslated region of the Xanthomonas resistance Xanthomonas resistance transcript, or all or part of the complement of both the coding sequence and the untranslated regions of a transcript encoding the Xanthomonas resistance polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. Patent Publication No. 20020048814, herein incorporated by reference.

iii. Double-Stranded RNA Interference

In some embodiments of the invention, inhibition of the expression of an Xanthomonas resistance polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of Xanthomonas resistance polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743, and WO 99/49029, WO 99/53050, WO 99/61631, and WO 00/49035; each of which is herein incorporated by reference.

iv. Hairpin RNA Interference and Intron-Containing Hairpin RNA Interference

In some embodiments of the invention, inhibition of the expression of an Xanthomonas resistance polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731; and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7, and U.S. Patent Publication No. 2003/0175965; each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295, and U.S. Patent Publication No. 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the Xanthomonas resistance polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362, and U.S. Pat. No. 6,635,805, each of which is herein incorporated by reference.

vi. Ribozymes

In some embodiments, the polynucleotide expressed by the expression cassette of the invention is catalytic RNA or has ribozyme activity specific for the messenger RNA of the Xanthomonas resistance polypeptide. Thus, the polynucleotide causes the degradation of the endogenous messenger RNA, resulting in reduced expression of the Xanthomonas resistance polypeptide. This method is described, for example, in U.S. Pat. No. 4,987,071, herein incorporated by reference.

vii. Small Interfering RNA or Micro RNA

In some embodiments of the invention, inhibition of the expression of Xanthomonas resistance polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference. For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of Xanthomonas resistance expression, the 22-nucleotide sequence is selected from an Xanthomonas resistance transcript sequence and contains 22 nucleotides of said Xanthomonas resistance sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

### 2. Polypeptide-Based Inhibition of Gene Expression

In one embodiment, the polynucleotide encodes a zinc finger protein that binds to a gene encoding an Xanthomonas resistance polypeptide, resulting in reduced expression of the gene. In particular embodiments, the zinc finger protein binds to a regulatory region of an Xanthomonas resistance gene. In other embodiments, the zinc finger protein binds to a messenger RNA encoding an Xanthomonas resistance polypeptide and prevents its translation.

Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 2003/0037355; each of which is herein incorporated by reference.

### 3. Polypeptide-Based Inhibition of Protein Activity

In some embodiments of the invention, the polynucleotide encodes an antibody that binds to at least one Xanthomonas resistance polypeptide, and reduces the activity of the Xanthomonas resistance polypeptide. In another embodiment, the binding of the antibody results in increased turnover of the antibody-Xanthomonas resistance complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

### 4. Gene Disruption

In some embodiments of the present invention, the activity of an Xanthomonas resistance polypeptide may be reduced or eliminated by disrupting the gene encoding the Xanthomonas resistance polypeptide. The gene encoding the Xanthomonas resistance polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have desired traits.

i. Transposon Tagging

In one embodiment of the invention, transposon tagging is used to reduce or eliminate the Xanthomonas resistance activity of one or more Xanthomonas resistance polypeptides. Transposon tagging comprises inserting a transposon within an endogenous Xanthomonas resistance gene to reduce or eliminate expression of the Xanthomonas resistance polypeptide. “Xanthomonas resistance gene” is intended to mean the gene that encodes an Xanthomonas resistance polypeptide.

In this embodiment, the expression of one or more Xanthomonas resistance polypeptides is reduced or eliminated by inserting a transposon within a regulatory region or coding region of the gene encoding the Xanthomonas resistance polypeptide. A transposon that is within an exon, intron, 5′ or 3′ untranslated sequence, a promoter, or any other regulatory sequence of an Xanthomonas resistance gene may be used to reduce or eliminate the expression and/or activity of the encoded Xanthomonas resistance polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540; and U.S. Pat. No. 5,962,764; each of which is herein incorporated by reference.

ii. Mutant Plants with Reduced Activity

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874; and Quesada, et al., (2000) Genetics 154:421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See, McCallum, et al., (2000) Nat. Biotechnol. 18:455-457, herein incorporated by reference.

Mutations that impact gene expression or that interfere with the function of the encoded protein are well known in the art. Insertional mutations in gene exons usually result in null-mutants. Mutations in conserved residues are particularly effective in inhibiting the activity of the encoded protein. Conserved residues of plant Xanthomonas resistance polypeptides suitable for mutagenesis with the goal to eliminate Xanthomonas resistance activity have been described. Such mutants can be isolated according to well-known procedures, and mutations in different Xanthomonas resistance loci can be stacked by genetic crossing. See, for example, Gruis, et al., (2002) Plant Cell 14:2863-2882.

In another embodiment of this invention, dominant mutants can be used to trigger RNA silencing due to gene inversion and recombination of a duplicated gene locus. See, for example, Kusaba, et al., (2003) Plant Cell 15:1455-1467.

The invention encompasses additional methods for reducing or eliminating the activity of one or more Xanthomonas resistance polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham, et al., (1999) Proc. Natl. Acad. Sci. USA 96:8774-8778; each of which is herein incorporated by reference.

The methods of the invention provides for improved plant performance such as stress tolerance, biomass accumulation or grain yield. This performance may be demonstrated in a number of ways including the following.

### Method of Use for Xanthomonas Resistance Polynucleotides, Expression Cassettes, and Additional Polynucleotides

The nucleotides, expression cassettes and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

In certain embodiments the nucleic acid sequences of the present invention can be used in combination (“stacked”) with other polynucleotide sequences of interest in order to create plants with a desired phenotype. The combinations generated can include multiple copies of any one or more of the polynucleotides of interest. The polynucleotides of the present invention may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,049); barley high lysine (Williamson, et al., (1987) Eur. J. Biochem. 165:99-106; and WO 98/20122); and high methionine proteins (Pedersen, et al., (1986) J. Biol. Chem. 261:6279; Kirihara, et al., (1988) Gene 71:359; and Musumura, et al., (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,736,514; 5,723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see U.S. Pat. No. 5,583,210), stalk strength, flowering time, or transformation technology traits such as cell cycle regulation or gene targeting (e.g., WO 99/61619; WO 00/17364; WO 99/25821), the disclosures of which are herein incorporated by reference.

In one embodiment, sequences of interest improve plant growth and/or crop yields.

For example, sequences of interest include agronomically important genes that result in improved primary or lateral root systems. Such genes include, but are not limited to, nutrient/water transporters and growth induces. Examples of such genes, include but are not limited to, maize plasma membrane H+-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359; both of which are herein incorporated by reference); and rice (Musumura, et al., (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser, et al., (1986) Gene 48:109); and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

### Genome Editing and Induced Mutagenesis

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein is generated using “custom” meganucleases produced to modify plant genomes (see, e.g., WO 2009/114321; Gao, et al., (2010) Plant Journal 1:176-187). Other site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41.

“TILLING” or “Targeting Induced Local Lesions IN Genomics” refers to a mutagenesis technology useful to generate and/or identify and to eventually isolate mutagenised variants of a particular nucleic acid with modulated expression and/or activity (McCallum, et al., (2000), Plant Physiology 123:439-442; McCallum, et al., (2000) Nature Biotechnology 18:455-457 and Colbert, et al., (2001) Plant Physiology 126:480-484). TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes.

TILLING also allows selection of plants carrying mutant variants. These mutant variants may exhibit modified expression, either in strength or in location or in timing (if the mutations affect the promoter, for example). These mutant variants may exhibit higher or lower activity than that exhibited by the gene in its natural form. TILLING combines high-density mutagenesis with high-throughput screening methods. The steps typically followed in TILLING are: (a) EMS mutagenesis (Redei and Koncz, (1992) In Methods in Arabidopsis Research, Koncz, et al., eds. Singapore, World Scientific Publishing Co, pp. 16-82; Feldmann, et al., (1994) In Arabidopsis. Meyerowitz and Somerville, eds, Cold Spring Harbor Laboratory Press, Cold Spring Harbor, N.Y., pp 137-172; Lightner and Caspar, (1998) In Methods on Molecular Biology 82:91-104; Martinez-Zapater and Salinas, eds, Humana Press, Totowa, N.J.); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (U.S. Pat. No. 8,071,840). Other mutagenic methods can also be employed to introduce mutations in a disclosed gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Embodiments of the disclosure reflect the determination that the genotype of an organism can be modified to contain dominant suppressor alleles or transgene constructs that suppress (i.e., reduce, but not ablate) the activity of a gene, wherein the phenotype of the organism is not substantially affected.

Hybrid seed production requires elimination or inactivation of pollen produced by the female parent. Incomplete removal or inactivation of the pollen provides the potential for selfing, raising the risk that inadvertently self-pollinated seed will unintentionally be harvested and packaged with hybrid seed. Once the seed is planted, the selfed plants can be identified and selected; the selfed plants are genetically equivalent to the female inbred line used to produce the hybrid. Typically, the selfed plants are identified and selected based on their decreased vigor relative to the hybrid plants. For example, female selfed plants of e are identified by their less vigorous appearance for vegetative and/or reproductive characteristics, including shorter plant height, small ear size, ear and kernel shape, cob color or other characteristics. Selfed lines also can be identified using molecular marker analyses (see, e.g., Smith and Wych, (1995) Seed Sci. Technol. 14:1-8). Using such methods, the homozygosity of the self-pollinated line can be verified by analyzing allelic composition at various loci in the genome.

Because hybrid plants are important and valuable field crops, plant breeders are continually working to develop high-yielding hybrids that are agronomically sound based on stable inbred lines. The availability of such hybrids allows a maximum amount of crop to be produced with the inputs used, while minimizing susceptibility to pests and environmental stresses. To accomplish this goal, the plant breeder must develop superior inbred parental lines for producing hybrids by identifying and selecting genetically unique individuals that occur in a segregating population. The present disclosure contributes to this goal, for example by providing plants that, when crossed, generate male sterile progeny, which can be used as female parental plants for generating hybrid plants.

A large number of genes have been identified as being tassel preferred in their expression pattern using traditional methods and more recent high-throughput methods. The correlation of function of these genes with important biochemical or developmental processes that ultimately lead to functional pollen is arduous when approaches are limited to classical forward or reverse genetic mutational analysis. As disclosed herein, suppression approaches provide an alternative rapid means to identify genes that are directly related to pollen development.

Promoters useful for expressing a nucleic acid molecule of interest can be any of a range of naturally-occurring promoters known to be operative in plants or animals, as desired. Promoters that direct expression in cells of male or female reproductive organs of a plant are useful for generating a transgenic plant or breeding pair of plants of the disclosure. The promoters useful in the present disclosure can include constitutive promoters, which generally are active in most or all tissues of a plant; inducible promoters, which generally are inactive or exhibit a low basal level of expression and can be induced to a relatively high activity upon contact of cells with an appropriate inducing agent; tissue-specific (or tissue-preferred) promoters, which generally are expressed in only one or a few particular cell types (e.g., plant anther cells) and developmental- or stage-specific promoters, which are active only during a defined period during the growth or development of a plant. Often promoters can be modified, if necessary, to vary the expression level. Certain embodiments comprise promoters exogenous to the species being manipulated. For example, the Ms45 gene introduced into ms45ms45 maize germplasm may be driven by a promoter isolated from another plant species; a hairpin construct may then be designed to target the exogenous plant promoter, reducing the possibility of hairpin interaction with non-target, endogenous promoters.

Exemplary constitutive promoters include the 35S cauliflower mosaic virus (CaMV) promoter promoter (Odell, et al., (1985) Nature 313:810-812), the maize ubiquitin promoter (Christensen, et al., (1989) Plant Mol. Biol. 12:619-632 and Christensen, et al., (1992) Plant Mol. Biol. 18:675-689); the core promoter of the Rsyn7 promoter and other constitutive promoters disclosed in WO 1999/43838 and U.S. Pat. No. 6,072,050; rice actin (McElroy, et al., (1990) Plant Cell 2:163-171); pEMU (Last, et al., (1991) Theor. Appl. Genet. 81:581-588); MAS (Velten, et al., (1984) EMBO J. 3:2723-2730); ALS promoter (U.S. Pat. No. 5,659,026); rice actin promoter (U.S. Pat. No. 5,641,876; WO 2000/70067), maize histone promoter (Brignon, et al., (1993) Plant Mol Bio 22(6):1007-1015; Rasco-Gaunt, et al., (2003) Plant Cell Rep. 21(6):569-576) and the like. Other constitutive promoters include, for example, those described in U.S. Pat. Nos. 5,608,144 and 6,177,611 and PCT Publication Number WO 2003/102198.

Tissue-specific, tissue-preferred or stage-specific regulatory elements further include, for example, the AGL8/FRUITFULL regulatory element, which is activated upon floral induction (Hempel, et al., (1997) Development 124:3845-3853); root-specific regulatory elements such as the regulatory elements from the RCP1 gene and the LRP1 gene (Tsugeki and Fedoroff, (1999) Proc. Natl. Acad., USA 96:12941-12946; Smith and Fedoroff, (1995) Plant Cell 7:735-745); flower-specific regulatory elements such as the regulatory elements from the LEAFY gene and the APETALA1 gene (Blazquez, et al., (1997) Development 124:3835-3844; Hempel, et al., supra, 1997); seed-specific regulatory elements such as the regulatory element from the oleosin gene (Plant, et al., (1994) Plant Mol. Biol. 25:193-205) and dehiscence zone specific regulatory element. Additional tissue-specific or stage-specific regulatory elements include the Zn13 promoter, which is a pollen-specific promoter (Hamilton, et al., (1992) Plant Mol. Biol. 18:211-218); the UNUSUAL FLORAL ORGANS (UFO) promoter, which is active in apical shoot meristem; the promoter active in shoot meristems (Atanassova, et al., (1992) Plant J. 2:291), the cdc2 promoter and cyc07 promoter (see, for example, Ito, et al., (1994) Plant Mol. Biol. 24:863-878; Martinez, et al., (1992) Proc. Natl. Acad. Sci., USA 89:7360); the meristematic-preferred meri-5 and H3 promoters (Medford, et al., (1991) Plant Cell 3:359; Terada, et al., (1993) Plant J. 3:241); meristematic and phloem-preferred promoters of Myb-related genes in barley (Wissenbach, et al., (1993) Plant J. 4:411); Arabidopsis cyc3aAt and cyc1At (Shaul, et al., (1996) Proc. Natl. Acad. Sci. 93:4868-4872); C. roseus cyclins CYS and CYM (Ito, et al., (1997) Plant J. 11:983-992); and 5 Nicotiana CyclinB1 (Trehin, et al., (1997) Plant Mol. Biol. 35:667-672); the promoter of the APETALA3 gene, which is active in floral meristems (Jack, et al., (1994) Cell 76:703; Hempel, et al., supra, 1997); a promoter of an agamous-like (AGL) family member, for example, AGL8, which is active in shoot meristem upon the transition to flowering (Hempel, et al., supra, 1997); floral abscission zone promoters; L1-specific promoters; the ripening-enhanced tomato polygalacturonase promoter (Nicholass, et al., (1995) Plant Mol. Biol. 28:423-435), the E8 promoter (Deikman, et al., (1992) Plant Physiol. 100:2013-2017) and the fruit-specific 2A1 promoter, U2 and U5 snRNA promoters from maize, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD zein protein, and the like. Additional tissue-specific promoters can be isolated using well known methods (see, e.g., U.S. Pat. No. 5,589,379). Shoot-preferred promoters include shoot meristem-preferred promoters such as promoters disclosed in Weigel, et al., (1992) Cell 69:843-859.

Use in Breeding Methods

The transformed plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This disclosure encompasses methods for producing a plant by crossing a first parent plant with a second parent plant wherein one or both of the parent plants is a transformed plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Transgenic plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

This invention can be better understood by reference to the following non-limiting examples. It will be appreciated by those skilled in the art that other embodiments of the invention may be practiced without departing from the spirit and the scope of the invention as herein disclosed and claimed.

### Examples

Rice plants that overexpress Xanthomonas resistance an AP2 like transcription factor exhibit significantly increased biomass and seed yield compared to wild-type plants, and this is especially true when grown under stressful conditions. This was derived through the analysis of a rice T-DNA insertion mutant possessing increased biomass and seed yield compared to wild-type plants. The presence of the T-DNA insertion was tracked across multiple generations, while recording biomass measurements to support the coloration of the insertion and the phenotype. Of the generations grown the mutant experienced as high as a 7.4-fold increase in biomass and a simultaneous 3.6-fold increase in seed yield compared to segregating wild-type plants. The insertion mutants also experience a delay in flowering time by an average of 16 days compared to wild-type plants, increasing their vegetative stage significantly which contributes to increased biomass. Insertion mutants also possess longer and wider leaves, and increased tiller girth compared to wild-type plants.

The insertion caused a mutagenic event that altered the expression and/or function of a nearby gene or genes. Further investigation via RT-PCR supported this claim, as the expression level of one of the neighboring genes is significantly up-regulated with the presence of the T-DNA insertion. This particular gene is a transcription factor belonging to a large superfamily of genes in plants. Further phenotypic investigation suggests that the degree of the phenotype (i.e. the increase in biomass) is influenced by abiotic and/or biotic stress. Plants placed under drought, pH, and salt stress are more successful in accumulating biomass, and producing seed with the over-expression of this particular transcription factor than wild type controls.

This transcription factor appears to have little to no expression in wild-type plants based on our preliminary analysis.

### Use in Breeding Methods

The transformed plants of the invention may be used in a plant breeding program. The goal of plant breeding is to combine, in a single variety or hybrid, various desirable traits. For field crops, these traits may include, for example, resistance to diseases and insects, tolerance to heat and drought, reduced time to crop maturity, greater yield, and better agronomic quality. With mechanical harvesting of many crops, uniformity of plant characteristics such as germination and stand establishment, growth rate, maturity, and plant height is desirable. Traditional plant breeding is an important tool in developing new and improved commercial crops. This invention encompasses methods for producing a plant by crossing a first parent plant with a second parent plant wherein one or both of the parent plants is a transformed plant according to the invention displaying Xanthomonas resistance as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids, and transformation. Often combinations of these techniques are used.

The development of hybrids in a plant breeding program requires, in general, the development of homozygous inbred lines, the crossing of these lines, and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed maize plant to an elite inbred line, and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross, or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly uniform; and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Transgenic plants of the present invention may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B)×(C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims. Thus, many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims.

### Example 1

The continuing battle between pathogens and their hosts has led to incredibly diverse virulence mechanisms in pathogens and counteracting defense mechanisms in their hosts. Pathogenic microbes and their host plants have followed a ‘zigzag’ course co-evolving new virulence strategies in pathogens and counteracting resistance mechanisms in hosts (1). Herein, Applicants identify a novel virulence mechanism by two bacterial pathogens that use iTALEs (interfering transcription activator-like effectors) to overcome disease resistance. iTALEs are truncated TALE variants expressed from the neglected “pseudogenes” in Xanthomonas oryzae pv. oryzae (Xoo) and X. oryzae pv. oryzicola (Xoc). Xa1, a NBS-LRR resistance gene, confers resistance against Xoo and Xoc by recognizing their multiple copies of TALEs with required nuclear localization of proteins. Xa1 homolog gene, Xa2, similarly functions to provide broad-spectrum resistance. However, iTALEs prevalent in the majority of pathogen isolates interfere with Xa1-mediated resistance and effectively limit the otherwise broad resistance. iTALEs require unique N-termini, truncated C-termini, and nuclear localization motifs for their suppressive activities. These findings reveal the pathogen ability to convert the type III effectors from resistance-trigger to resistance-interferer in microbe/host interaction and elucidate novel targets for disease intervention.

### Material and Methods:

### Plant Material, Bacterial Strains, Medium, and Growth Conditions

Seeds of all rice varieties were kindly provided by the International Rice Research Institute, the U.S. National Small Grains Collection, and collaborators. All plants were grown in growth chambers with photoperiod of 12 hr, temperature of 28° C. daytime and 26° C. at night. Escherichia coli strains were grown on Luria-Bertani medium supplemented with appropriate antibiotics at 37° C. All Xanthomonas oryzae pv. oryzae (Xoo) and X. o. pv. oryzicola (Xoc) strains were grown at 28° C. in nutrient broth with agar (NA) (1% polypeptone, 0.5% yeast extract, 1% sucrose, and 1.5% agar), nutrient broth without agar (NB), NA without sucrose (NAN), NA with 10% sucrose (NAS) or TSA (10 g/L tryptone, 10 g/L sucrose, 1 g/L glutamic acid). Antibiotics were used at the following concentration if required: cephalexin, 10 μg/ml; kanamycin, 25 μg/ml; ampicillin, 100 μg/ml and spectinomycin, 100 μg/ml. Strains and plasmids used in this study are listed in Table 1.

TABLE 1
Bacterial strains used in this study

| | | Reference/ |
|---|---|---|
| Strains or Plasmids | Relevant characteristics | Source |
| Strains | | |
| Xanthomonas | | |
| oryzae pv. oryzae | | |
| PXO99 | Philippine race 6 | (4) |
| PA | Tal7 and Tal8 cluster knock-out mutant of PXO99 | This study |
| PB | Tal3 cluster knock-out mutant of PA | This study |
| PC | Tal2 cluster knock-out mutant of PB | This study |
| PD | Tal9 cluster knock-out mutant of PC | This study |
| PE | Tal5 cluster knock-out mutant of PD | This study |
| PF | Tal1 cluster knock-out mutant of PE | This study |
| PG | Tal6 cluster knock-out mutant of PF | This study |
| PH | Tal4 cluster knock-out mutant of PG | This study |
| ΔTal3 | Tal3 cluster knock-out mutant of PXO99 | This study |
| Xanthomonas | | |
| oryzae pv. oryzicola | | |
| RS105 | Chinese strain | (5) |
| ΔTal5e | Tal5e knock-out mutant of RS105 | This study |
| Plasmids | | |
| pHZWpthXo1 | pHM1 expressing pthXo1 under lacZ promoter in pZW | (6) |
| pHZWtal3aC | pHM1 expressing 6126bp ClaI fragment isolated from | This study |
| | PXO99 containing tal3a in pBluescript KS(−) | |
| pHZWtal3bC | pHM1 expressing 4547bp ClaI fragment isolated from | This study |
| | PXO99^{A} containing tal3b in pBluescript KS(−) | |
| pHZWtal3aF | pHM1 expressing tal3a under lacZ promoter with FLAG | This study |
| | in pZW | |
| pHZWtal3bF | pHM1 expressing tal3a under lacZ promoter with FLAG | This study |
| | tag in pZW | |
| pHZWΔ1-15 | tal3a repeat-deletion derivative in pHZW | This study |
| pHZWΔ1-10 | tal3a repeat-deletion derivative in pHZW | This study |
| pHZWΔ1-2 | tal3a repeat-deletion derivative in pHZW | This study |
| pHZWtal3aFL | Full-length tal3a in pHZW | This study |
| pHZWtal3aM | tal3a with its NLS mutated in pHZW | This study |
| pHZWtal3aSV | tal3a with its NLS mutated and additional SV40 NLS | This study |
| pHZWtal3bFL | Full-length tal3b in pHZW | This study |
| pHZWtal3bM | tal3b with its NLS mutated in pHZW | This study |
| pHZWtal3bSV | tal3b with its NLS mutated and additional SV40 NLS | This study |
| pHMZWpthXo1M | PthXo1 with NLS mutated | This study |
| pHMZWpthXo1SV | PthXo1 with NLS mutated and additional SV40 NLS | This study |
| pHMZWavrXa7 | AvrXa7 from pZWavrXa7 in pHM1 | (1) |
| pHMZWavrXa7M | AvrXa7 with NLS mutated | (1) |
| pHMZWavrXa7SV | AvrXa7 with NLS mutated and additional SV40 NLS | (1) |
| pHZWXo1N-Tal3aRC | tal3a variant containing PthXo1 N-terminus in pHZW | This study |
| pHZWTal3aNR-Xo1C | tal3a variant containing PthXo1 C-terminus in pHZW | This study |
| pHZWtal4 | pHM1 expressing tal4 from PXO99 in pZW | This study |
| pHZWtal9d | pHM1 expressing tal9d from PXO99 in pZW | This study |
| pHZWtal3_PXO86 | pHM1 expressing tal3 from PXO86 in pZW | This study |
| pHZWtal6_PXO86 | pHM1 expressing tal6 from PXO86 in pZW | This study |
| pHZWtal5e_RS105 | pHM1 expressing tal5e from RS105 in pZW | This study |
| pHM1tal12_BXOR1 | pHM1 expressing tal12 from BXOR1 | This study |
| pHM1tal11h_BXOR1 | pHM1 expressing tal11h from BXOR1 | This study |

### TALE Gene Cluster Deletion

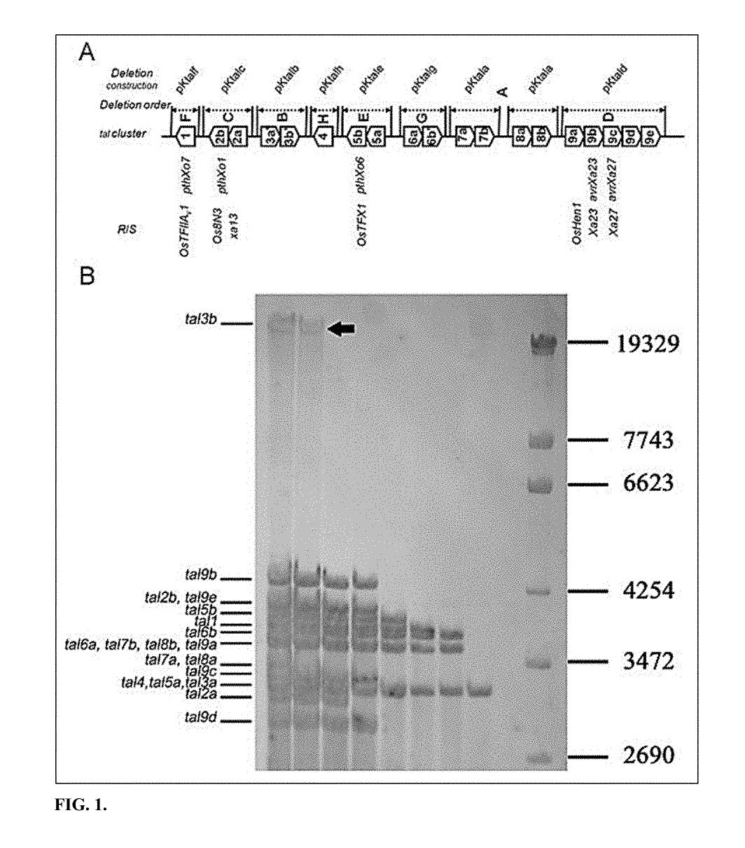

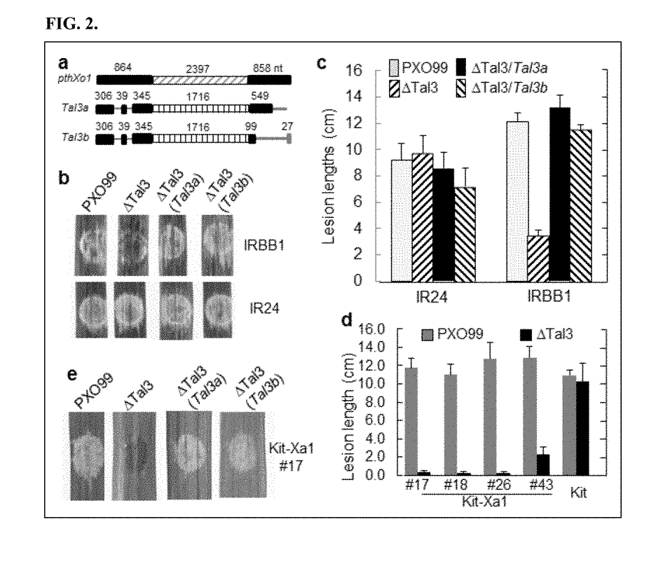

Suicide vector pKMS1 was used to generate PXO99 gene cluster deletion mutants using a method as described (1). Nine clusters of TALE genes were sequentially deleted from PXO99 as indicated in FIG. 1. Because DNA sequences of TALE genes are nearly identical, unique sequences flanking each TALE cluster were chosen for knockouts. Based on the PXO99 genome sequence (NCBI accession, CP000967), two pairs of primers, ΦF1/ΦR1 and ΦF2/ΦR2 (Φ represents a TALE gene cluster), were used to amplify the upstream and downstream regions flanking the target TALE loci by using the PXO99 genomic DNA as the template (primer information is provided in Table 2). The two PCR products for each cluster deletion were restricted accordingly and cloned into the pKMS1 multiple cloning sites and confirmed by sequencing for accuracy. The first round of mutagenesis was carried out on PXO99 with pKtala targeting the duplicated clusters Tal7 and Tal8. Plasmid was transferred into the competent cells of PXO99 through electroporation and the transformants were plated on the kanamycin NA without sucrose plates. Single colonies were transferred to NB without sucrose medium and incubated with shaking for 12 hr at 28° C. Then cells were plated on NA with 10% sucrose. Sucrose tolerant colonies were duplicated on NA and NA with kanamycin plates. The kanamycin sensitive colonies were screened by PCR (using primers Tal7/8F1&Tal7/8R2) and Southern Blot to verify the deletion of the gene clusters 7 and 8 (FIG. 1). The mutant was used for the second round of mutagenesis with construct pKtalb targeting the cluster 3 of pseudogenes. Similarly, sequential deletions were performed to complete the deletions of all 9 TALE gene clusters (FIG. 1).

For Southern Blot, genomic DNAs of PXO99 and its derived TALE mutants were extracted using the AxyPrep Bacterial Genomic DNA Miniprep Kit (Axygen, Hanzhou, China). DNA samples (3 μg) were digested with BamHI at 37° C. for 4 h, separated in 1.2% agarose gel through electrophoresis, and transferred to Hybond N⁺ nylon membranes (Millipore, Billerica, U.S.A). The probe was a DIG-labeled 1368 bp SphI fragment containing the repetitive sequence of avrXa3 (GenBank accession no. AY129298.1). Labeling, hybridization and detection procedures were performed by following the manufacturer's instruction (Roche, Sweden).

Tal5e deletion strain of RS105 was similarly produced. Primers Tal5RSF1&Tal5RSR1 and Tal5RSF2&Tal5RSR2 were used to generate the two homologous fragments for deletion of Tal5e.

### DNA Manipulation and Plasmid Construction

DNA manipulation and PCR were conducted according to standard protocols (2). Plasmids were introduced by electroporation into Xanthomonas and E. coli bacterial cells as described previously (3). Oligonucleotide primers for PCR were synthesized by Invitrogen Biotechnology Co., Ltd. (Shanghai, China) and Integrated DNA Technologies (Coralville, Iowa, USA); PCR was performed with Ex-Taq (TakaRa Biotechnology, Dalian, China) and Phusion High-Fidelity DNA Polymerase (New England BioLabs, Ipswich, Mass., USA).

Construction of genomic libraries for Tal3a, Tal3b and other pseudogenes was completed as follows. Genomic DNA of PXO99 was digested with ClaI and separated in 1% agarose gel. DNA fragments of ˜6.5-4 kb were purified from the agarose gel and ligated into the ClaI restricted pBluescript KS+ (Stratagene, La Jolla, Calif., USA). The ligation reaction was mobilized into E. coli DH5α cells. The library was screened for Tal3a and Tal3b using probe derived from the SphI fragment (repetitive region) of avrXa3. Candidate clones were sequenced for confirmation of Tal3a and Tal3b. To isolate the pseudogenes from PXO86 and RS105, genomic DNA was digested with BamHI and appropriate DNA fragments were purified from the agarose gel. The DNA fragments were subcloned into BamHI-digested pBluescript KS+ and transferred into DH5a cells for screening of positive clones of pseudogenes Tal3 and Tal6 of PXO86 and Tal5e of RS105.

To construct the FLAG epitope tagged Tal3a and Tal3b, primers Tal3aHFF & Tal3aHFR and Tal3aHFF & Tal3bHFR were used to amplify the 3′ regions of Tal3a and Tal3b, respectively. The purified PCR products were first digested using HincII and HindIII and then along with BamHI-HincII fragments of Tal3a and Tal3b, ligated into backbone of pZWavrXa7 (BamHI-HindIII digested), resulting in pZWTal3aF and pZWTal3bF, respectively. Both pZWTal3aF and pZWTal3bF were restricted with HindIII and ligated into pHM1 (HindIII digested) to generate pHZWTal3aF and pHZWTal3bF.

For construction of the internal central repeat deletions, pZWTal3aF was first completely digested with AatII, then partially with MscI, fragments in a range of 200 to 1,800 bp were recovered and ligated back to pZWTal3aF (restricted with MscI-AatII). Clones with various sizes of repeat regions were selected and sequenced to confirm the accuracy of deletions.

For domain swapping of avrXa7, avrXa10 and pthXo1 into Tal3a, the respective SphI central repetitive region of each gene was used to replace the corresponding region of Tal3a, resulting in pZWavrXa7a, pZWavrXa10a and pZWpthXo1a. The resulting plasmids were ligated into pHM1 at the HindIII restriction sites.

The full-length versions of Tal3a and Tal3b were constructed as following. The N-terminal and central repetitive domain coding regions were obtained with PstI and AatII from Tal3a and Tal3b, then swapped into the corresponding region of pZWavrXa7, resulting in pZWTal3aFL and pZWTal3bFL, respectively. The resultant plasmids were individually ligated into pHM1 with HindIII restriction.

The chimeric Tal3a with the N-terminus coding region of pthXo1 was constructed by cloning the BlpI-HindIII fragment from pZWTal3aF into the corresponding region of pZWpthXo1. Similarly, BlpI-HindIII fragment of Tal3a was swapped into pZWavrXa7, resulting in gene encoding N-terminus of AvrXa7 and the repetitive and C-terminal domains of Tal3a. Both pZW versions were subcloned into pHM1 by HindIII restriction.

To construct the nuclear localization signal (NLS) mutant of Tal3a, primers Tal3aM-F1&Tal3aM-R along with pZWTal3aF as template were used for the first round of PCR; the amplicon was used for the second round of PCR with primers Tal3aM-F2&Tal3aM-R. One mutation was incorporated into Tal3a in each round of PCR. The final PCR product was cloned back into pZWTal3aF by EcoRI and HindIII restriction followed by ligation, resulting in pZWTal3aM. Primers Tal3aM-F2&Tal3aMSV-R along pZWTal3aM as template were used to add the SV40 NLS coding sequence into Tal3aM through PCR approach and subsequently cloning through EcoRI/HindIII restriction and ligation. Similarly, Tal3bM (NLS mutant) was constructed. Primers Tal3HincII-F&Tal3bM-R along pZWTal3bF as template were used to incorporate NLS mutant sequence via PCR approach. The PCR amplicon was digested with HincII and HindIII and ligated back into pZWTal3bF, resulting in pZWTal3bM. The addition of SV40 NLS coding sequence was carried out using PCR with primers Tal3HincII-F&Tal3bSV-R plus Tal3bM as template, followed by restriction of HincII/HindIII and DNA ligation, resulting in pZWTal3bSV. The resulting plasmids were sequenced for the accuracy of PCR amplification regions. All pZW versions of plasmids were ligated into pHM1 through HindIII restriction and ligation.

GFP-tagged Tal3a and Tal3b were constructed using PCR approach. Primers GFPKp-F and GFPBam-R along with an eGFP template were used to PCR-amplify the GFP coding region. The PCR product cloned into pGEM-T vector through A/T cloning and sequenced for accuracy. The eGFP coding region was cut out with KpnI and BamHI. The restricted eGFP DNA fragment along with BamHI-HindIII fragments of Tal3aF, Tal3bF and Tal3bM was ligated under the CaMV 35S promoter and Nos terminator in pUC19 (restricted by KpnI and HindIII).

Gene encoding the PthXo1 nuclear localization signal (NLS) mutation was constructed by swapping the whole 3′ region (813 bp) downstream of AatII site in pZWpthXo1 with a gBlock synthesized from the Integrated DNA Technologies (Coralville, Iowa, USA). The gBlock encoding the three NLS mutations was used to replace the corresponding region of pthXo1 at AatII and HindIII sites using Gibson cloning method. Similarly, gBlock encoding the NLS mutations and additional SV40 NLS was swapped into the corresponding region of pthXo1 in pZWpthXo1. The NLS mutation and addition of SV40 NLS for avrXa7 in pZWavrXa7M123 (referred to as avrXa7M) and pZWavrXa7SV40 (referred to as avrXa7SV), respectively, were described (17). The pZW versions of pthXo1 each were subcloned into pHM1 at the HindIII restriction sites.

To clone iTALE genes Tal11h and Tal112 from BXOR1, primers BXOR1F and BXOR1R that are complementary to the flanking regions of both genes were used to amplify the respective fragments from the genomic DNA. The amplicons were cloned into pHM1 (BamHI digested) directly through Gibson cloning. The accuracy of cloning was confirmed via DNA sequencing.

### Transient Gene Expression and Microscopy

The mesophyll protoplasts of rice cultivar Kitaake were isolated and transfected as described (34). Rice protoplasts transfected with eGFP-Tal3a, eGFP-Tal3b and its NLS mutant were observed 36 hours post transfection using a Leica SP5×MP confocal/multiphoton microscope at the ISU Confocal and Multiphoton Facility. Fluorescence images were acquired at 522-572 nm (eGFP) and 358-461 nm (DAPI).

### Genotyping of Xoo Strains for Presence of Pseudogenes

Primers Tal3aF1&Tal3aR1 and Tal3bF1&Tal3bR1 were used along with the genomic DNA of individual strains for detection of the Tal3a and Tal3b type pseudo TALE genes, respectively.

### Reverse Transcription Polymerase Chain Reaction (RT-PCR)

Bacterial RNA was extracted using TRI Reagent Solution (ThermoFisher Scientific, Waltham, Mass., USA) according to the manufacturer's instruction. One microgram of total RNA was treated with DNase I (ThermoFisher Scientific, Waltham, Mass., USA) to eliminate the DNA contamination and used for cDNA synthesis by using iScript cDNA Synthesis kit (Bio-Rad, Hercules, Calif., USA) with random 9-mers following the user's manual. cDNA derived from 50 ng of RNA was used for each reaction of semi-quantitative PCR. Semi-qPCR for Tal3a and Tal3b gene expression in PXO99 was performed by using gene specific primers Tal3aF1&Tal3aR1 and Tal3bF1&Tal3bR1, respectively. Ribosomal 16S RNA expression was used as an internal control with gene specific primers (16SrRNA-F and 16SrRNA-R).

For plant transcript detection, RNA was extracted from leaves inoculated with bacteria as specified in the text. One microgram of total RNA was first treated with DNase I (Invitrogen) and used for cDNA synthesis by using the iScript cDNA Synthesis kit (Bio-Rad). cDNA derived from 0.025 g of total RNA was used for each real-time PCR, which was performed on Strategene's Mx4000 multiplex quantitative PCR system using iQ SYBR green Supermix (Bio-Rad). The gene-specific primer sequences are provided in Table 2. The average threshold cycle (Ct) was used to determine the fold change of gene expression. As an internal control, rice action gene was used. The 2^{ΔΔCt} method was used for relative quantification (35).

TABLE 2
Primers and sequence information.

| Primer name | Primer Sequence (5′ to 3′) |
|---|---|
| Tal1F1 (SEQ ID NO: 34) | ATATATCTAGACGGCAGTGATGGCGAACGGTT |
| Tal1R1 (SEQ ID NO: 35) | ATATAGAGCTCTCGTTGGCCAGGCGCAGCTCG |
| Tal1F2 (SEQ ID NO: 36) | ATATAGAGCTCGCCGGCGACATCGCCCACCGC |
| Tal1R2 (SEQ ID NO: 37) | TATATGTCGACAAAGGTCCGTGCGGCATCTGG |
| Tal2F1 (SEQ ID NO: 38) | TATATCCCGGGATGCTGGCGGCCAGTA |
| Tal2R1 (SEQ ID NO: 39) | TATATAAGCTTCATGCATTCGCCGATT |
| Tal2F2 (SEQ ID NO: 40) | TATATAAGCTTCACTGCCTCCACTGCG |
| Tal2R2 (SEQ ID NO: 41) | TATATGTCGACCCACATCTGCGGCGCA |
| Tal3F1 (SEQ ID NO: 42) | ATACCCGGGCATGGCGGAATCCGGTGCG |
| Tal3R1 (SEQ ID NO: 43) | AATACTAGTAATCTTGAGAAGTTGGCCTG |
| Tal3F2 (SEQ ID NO: 44) | ATAACTAGTTCGCATGATTGATGGAGCTA |
| Tal3R2 (SEQ ID NO: 45) | TTAGCATGCTCGTACGCATGAAGGCTGGA |
| Tal4F1 (SEQ ID NO: 46) | ATATACCCGGGATGCATTTTTTGGCGAAGGGCACT |
| Tal4R1 (SEQ ID NO: 47) | ATATATCTAGAACATCCGCTGGTTGCTGCGGGCCA |
| Tal4F2 (SEQ ID NO: 48) | ATATATCTAGAGTGGACCTGCTCAAGCGAATGATG |
| Tal4R2 (SEQ ID NO: 49) | TATATGTCGACTTCTGGCGCAACTTCGGCCAGGCA |
| Tal5F1 (SEQ ID NO: 50) | ATATACCCGGGAGCAATGGCCGCATGAGCCAGG |
| Tal5R1 (SEQ ID NO: 51) | TATATGCATGCGCCGCCGCAAGCGCCGTCGGCG |
| Tal5F2 (SEQ ID NO: 52) | TGCGCCATGCATGCACTGCCTCCACTGCGGTCA |
| Tal5R2 (SEQ ID NO: 53) | TATATGTCGACCACAATCAATGGCCTGCTGGGC |
| Tal6F1 (SEQ ID NO: 54) | ATATACCCGGGATGGCAATGAGATATGGTTGAACC |
| Tal6R1 (SEQ ID NO: 55) | TATATGGATCCTCACCGCTGAAAGTGCGTGCTAAT |
| Tal6F2 (SEQ ID NO: 56) | ATATAGGATCCGATCCTGGTACGCCCATCGCTGCC |
| Tal6R2 (SEQ ID NO: 57) | TATATGTCGACCGCAGCAAGCAGCGCTTGTGGAC |
| Tal7/8F1 (SEQ ID NO: 58) | GGACCCGGGGTAGGGACCACAGACCGCTAG |
| Tal7/8R1 (SEQ ID NO: 59) | CCAAAGCTTACTGTCGAACGCACCTTCGGT |
| Tal7/8F2 (SEQ ID NO: 60) | TGGAAGCTTGACCTTGATGCGCCTAGCC |
| Tal7/8R2 (SEQ ID NO: 61) | TCCTCTAGACTGAGGCAATAGCTCCATC |
| Tal9F1 (SEQ ID NO: 62) | ATATACCCGGGATGCTCAAGAACGATCGCCTGCTG |
| Tal9R1 (SEQ ID NO: 63) | TATATGCATGCACCCGAATCCTGGGTGACACGGGC |
| Tal9F2 (SEQ ID NO: 64) | ATATAGCATGCATTTTTCACCACTTCTGAGAAGCG |
| Tal9R2 (SEQ ID NO: 65) | TATATGTCGACCCTTGCCGAGAGTTCAAGACCTGG |
| Tal3aHFF (SEQ ID NO: 66) | CGTTGGCCGCGTTGACCAACGACCACCTCGT |
| Tal3aHFR SEQ ID NO: 67) | TATAAGCTTCACTTATCGTCATCGTCCTTGTAATCGGACCGTT |
| | TACGTCTGCTTG |
| Tal3bHFF (SEQ ID NO: 68) | CGTTGGCCGCGTTGACCAACGACCAACTCGT |
| Tal3bHFR (SEQ ID NO: 69) | TATAAGCTTCACTTATCGTCATCGTCCTTGTAATCATCATGCG |
| | ATTTCCTCTTCCTTGAAT |
| Tal5RSF1 (SEQ ID NO: 70) | CGACCCGGGGCACCCGTGTCACG |
| Tal5RSR1 (SEQ ID NO: 71) | ATGGATCCTGGCGCATCGCCATCGCCGCTATGG |
| Tal5RSF2 (SEQ ID NO: 72) | TGGGATCCATCAGGCATACCTCTTTGGAGAA |
| Tal5RSR2 (SEQ ID NO: 73) | ATGTCGACTCATGCTGCACACCAAGCCGTGG |
| BX0R1F (SEQ ID NO: 74) | CGGAGGGGTTGGATCCTACGACACGCATCGGTAGATCTG |
| BX0R1R (SEQ ID NO: 75) | CGAGGGCCCGGGATCCGTCGCTCAGATAGTCCCCCGA |
| Tal3aF1 (SEQ ID NO: 76) | CAGACGTAAACGGTCCT |
| Tal3aR1 (SEQ ID NO: 77) | ACGCTGCCAGGTCGGCAACC |
| Tal3bF1 (SEQ ID NO: 78) | GACGTCCTGCCCCGCATT |
| Tal3bR1 (SEQ ID NO: 79) | GGACGTCGCTCAGATAGTC |
| 16SrRNA-F (SEQ ID NO: 80) | TGGTAGTCCACGCCCTAAACG |
| 16SrRNA-R (SEQ ID NO: 81) | CTGGAAAGTTCCGTGGATGTC |
| Xa1F1 (SEQ ID NO: 82) | TGATTACGAATTCGAGCTAACAACTTTTCTTTTTCTGAATC |
| Xa1R1 (SEQ ID NO: 83) | TCATTACCAAAAGCATGCACTTTAAATAGTGA |
| Xa1F2 (SEQ ID NO: 84) | TGGTCACTATTTAAAGTGCATGCTTTTGGTAA |
| Xa1R2 (SEQ ID NO: 85) | TAGAGGATCCCCGGGTACCGTGACAATGCATTGGAGCGGATT |
| Xa1F3 (SEQ ID NO: 86) | AACTGATTACTCGGTGGCTTG |
| U (SEQ ID NO: 87) | TGTAAAACGACGGCCAGT |
| PR1F (SEQ ID NO: 88) | CGTCTTCATCACCTGCAA |
| PR1R (SEQ ID NO: 89) | TCAGCGTACGATAGTAGTA |
| PBZ-F (SEQ ID NO: 90) | CTCAAGATGATCGAGGAC |
| PBZ-R (SEQ ID NO: 91) | CGTCTTCATCACCTGCAA |
| PDX-F (SEQ ID NO: 92) | ACGACATAAACGGGCCAC |
| PDX-R (SEQ ID NO: 93) | AGGTGCTAATGCCATGGCT |
| Actin-F (SEQ ID NO: 94) | CTCAGCACATTCCAGCAGAT |
| Actin-R (SEQ ID NO: 95) | ACAGATAGGCCGGTTGAAAA |

### Rice Transformation

For construction of Xa1, primers Xa1F1/R1 and Xa1F2/R2 were used to amplify two fragments from Xa1 locus in IRBB1. The two overlapping amplicons were joined and inserted into SacI site in pCAMBIA1300 using Gibson Assembly Master Mix (New England BioLabs, Ipswich, Mass., USA). Xa1 with ubiquitin promoter was constructed with a synthetic DNA fragment (751 bp) of 5′ end and a PCR-amplicon (4664 bp) of 3′ end of Xa1 under the rice ubiquitin 2 promoter in pCAMBIA1300. Both cDNA pUbi:Xa1 and genomic clone p1300-Xa1 were electroporated into Agrobacterium tumefaciens strain EHA 105. Genomic region of Xa2 was PCR-amplified with primers (Xa2F1/R1) and genomic DNA of IRBB2. The amplicon was cloned into pCAMBIA1300 at EcoRI and HinduII through Gibson cloning. Calli from immature embryos of Kitaake were initiated and transformed by using Agrobacterium 5 tumefaciens as described (36). Transgenic plants were genotype with primers (Xa1F3 and U) and (Xa2F and U) located at the 3′ of Xa1 and Xa2 and in backbone of pCAMBIA1300, respectively.

### Disease Assays

Hypersensitive cell death response (HR) and virulence assays were conducted as descried previously (37). Briefly Xoo strains were grown in NB with appropriate antibiotics at 28° C. Bacterial cells were collected from culture by low-speed (4000 rpm) centrifugation, washed twice and suspended in sterile water. The suspensions were adjusted to an optical density of 0.5 at 600 nm, and were used to infiltrate into leaves of rice seedlings (about 3 weeks old) with the needleless syringe to assess the strain ability to trigger HR in plants. The cells of the same concentration were also used to inoculate the fully expanded leaves of adult plants (about 2 months old) using the leaf-tip clipping method to evaluate the strain ability to cause disease or trigger resistance in plants by measuring the lesion lengths. Similarly, inoculum of Xoc was infiltrated into rice leaves using the needleless syringe to measure the rice reactions (susceptible or resistant). One-way analysis of variance (ANOVA) statistical analyses were performed on all measurements. The Tukey honest significant difference test was used for post-ANOVA pair-wise tests for significance, set at 5% (P<0.05).

### Results

### Deletion Mutant of Tal3 Cluster of “Pseudogenes” Triggers Disease Resistance

PXO99, a representative strain of X. o. pv. oryzae, is virulent in a large number of rice varieties and contains nine gene clusters totaling nineteen individual TALE genes, some of which are important pathogenesis factors in bacterial blight of rice (15-18). We generated a series of PXO99 mutant strains that are depleted of different and complete complements of TALE genes by sequentially deleting individual TALE gene clusters (FIG. 1). Disease assays with those mutants on thirty-six rice varieties of different genetic background were performed to assess the pathogenesis role of each gene cluster. In agreement with prior study (17), mutant of PXO99 with deletion of pthXo1-containing cluster lost the ability to cause disease in compatible rice varieties. To our surprise, mutants with deletion of the cluster 3 (Tal3a/Tal3b) started to show resistance in two rice varieties IRBB1 and Kogyoku but not in other rice lines all susceptible to PXO99 (Table 3).

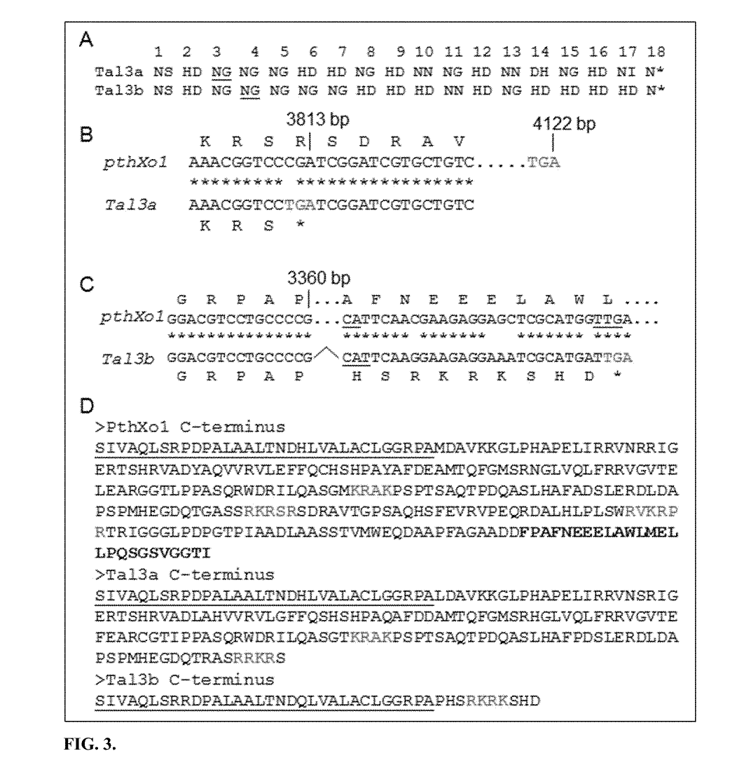

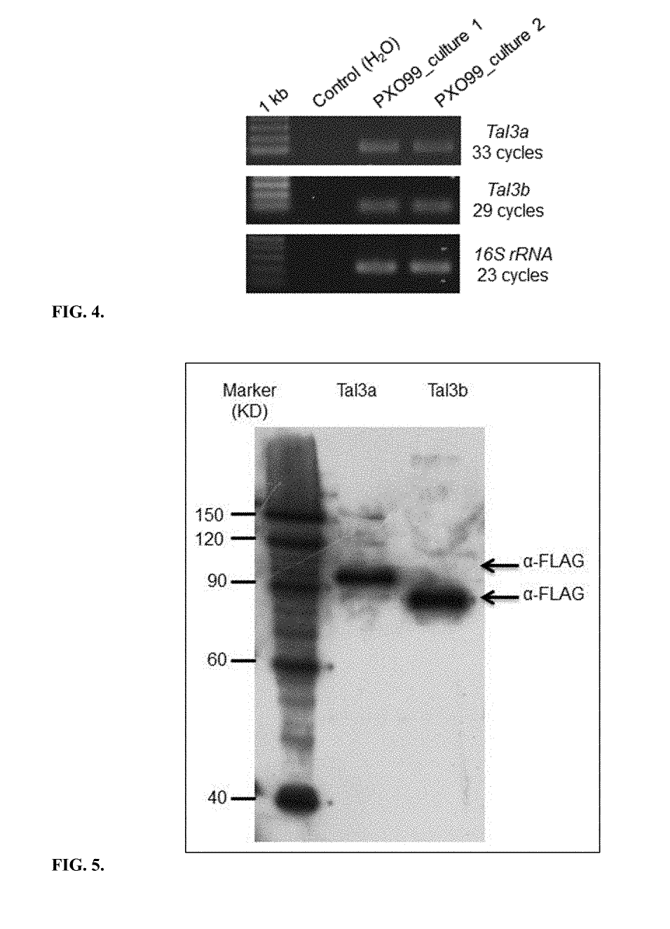

PXO99 genome contains three TALE “pseudogenes” that have been annotated and previously reported (18). Tal6b has a 1-bp insertion at the 97 bp position in the 5′ coding sequence. Tal3a carries a premature stop codon due to a C to T change at the 3013 bp position of the gene, probably encoding a protein with a C-terminal truncation of 103 amino acids; while Tal3b undergoes a large deletion (688 bp) at the 2560 bp position relative to Tal3a and presumably encodes a product with 254 amino acids deleted and 10 additional amino acids due to a frame-shift (18). Both genes contain two deletions (129 bp and 45 bp) within the 5′ regions (FIG. 2A, and FIG. 3). Tal3a and Tal3b, if expressed, are predicted to contain identical N-termini, distinct central repetitive and C-terminal domains; both effectors contain the nuclear localization motifs but lack the transcriptional activation domains (FIG. 3). Indeed, reverse transcription polymerase chain reaction (RT-PCR) on bacterial RNA revealed the expression of both “pseudogenes” (FIG. 4).

iTALE Tal3a and Tal3b are Virulence Factors

A mutant of PXO99 was constructed with only the cluster 3, containing the two TALE “pseudogenes”, deleted (ΔTal3) to assess the role of the “pseudogenes” in pathogenesis with other 17 TALE genes intact. ΔTal3 triggered hypersensitive cell death response (HR) in IRBB1 but not in IR24 when injected directly into the leaf blade (FIG. 2B). Similarly, ΔTal3 was able to cause disease in IR24 but not in IRBB1 on the basis of lesion length when the bacteria were introduced at the leaf tip (FIG. 2C). Tal3a and Tal3b were cloned and introduced, with an added FLAG epitope, individually to ΔTal3. Each clone enabled ΔTal3 to cause disease in IRBB1 comparable to the parent strain PXO99 (FIG. 2B, 2C). Western blotting probed with the anti-FLAG antibody showed the presence of Tal3a and Tal3b in the complementing strains of ΔTal3 (FIG. 5). The results indicate that the TALEs Tal3a and Tal3b expressed from the previously annotated and neglected “pseudogenes” function as TAL effector variants in PXO99 for virulence by interfering with the host resistance in IRBB1. Both effector variants and their relatives are referred to hereinafter as iTALEs (interfering TAL effectors).

iTALEs Interfere with Xa1-Mediated Resistance in Rice

IRBB1 and IR24 are near isogenic rice lines for the R gene Xa1 (19), which was identified as a NBS-LRR type R gene from Kogyoku and IRBB1 with no cognate elicitor (or avirulence) gene identified yet (20). To test if the resistance to ΔTal3 and the suppressive effect of Tal3a and Tal3b were, in fact, specific to Xa1 and not due to another gene in the IRBB1 background, the Xa1 locus was PCR-amplified from IRBB1 and transferred into the rice cultivar Kitaake, which is susceptible to PXO99 and ΔTal3. Xa1 transgenic lines (n=7) were susceptible to PXO99, but were resistant to ΔTal3 in terms of HR and lesion length, and the resistance were reversed by introduction of either Tal3a or Tal3b to ΔTal3 (FIGS. 2D, 2E). The results demonstrate that PXO99 gains virulence by deploying its iTALE genes Tal3a and Tal3b to mask the otherwise resistance in Xa1-containing plants.

Suppression of Xa1-Mediated Resistance by iTALEs Requires their N- and C-Terminal Structures and Nuclear Localization Motifs.

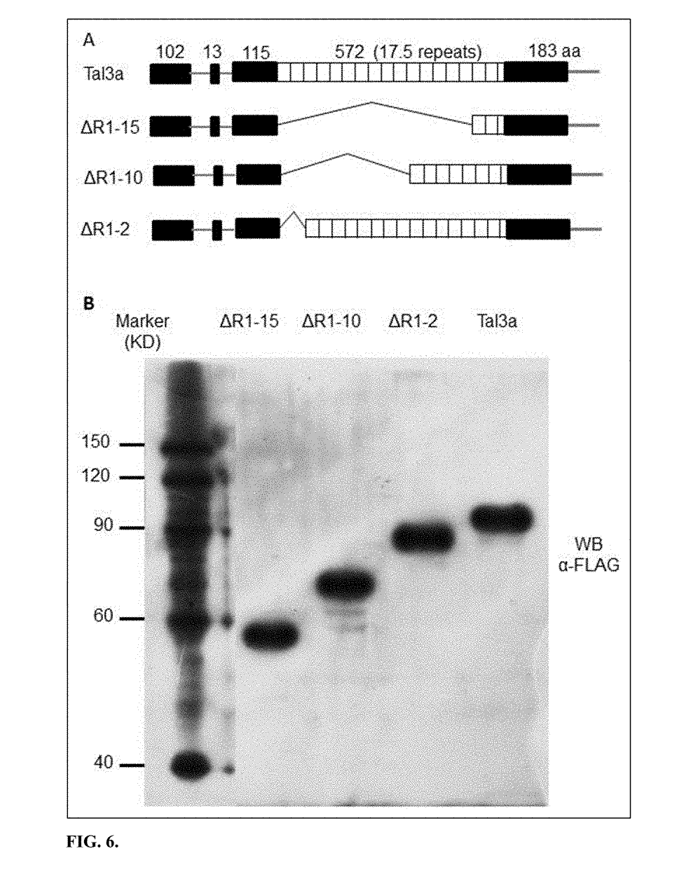

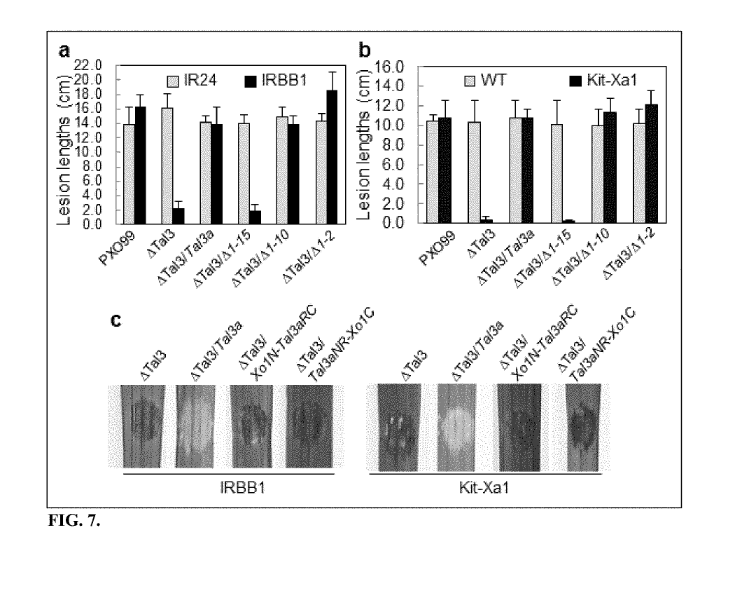

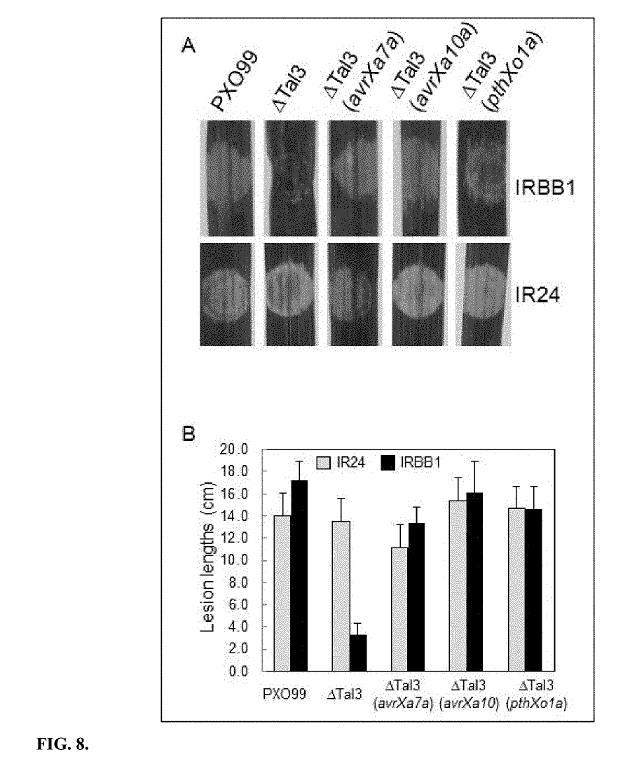

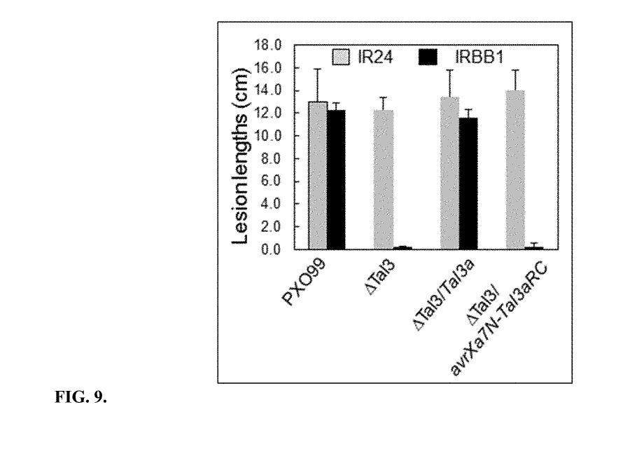

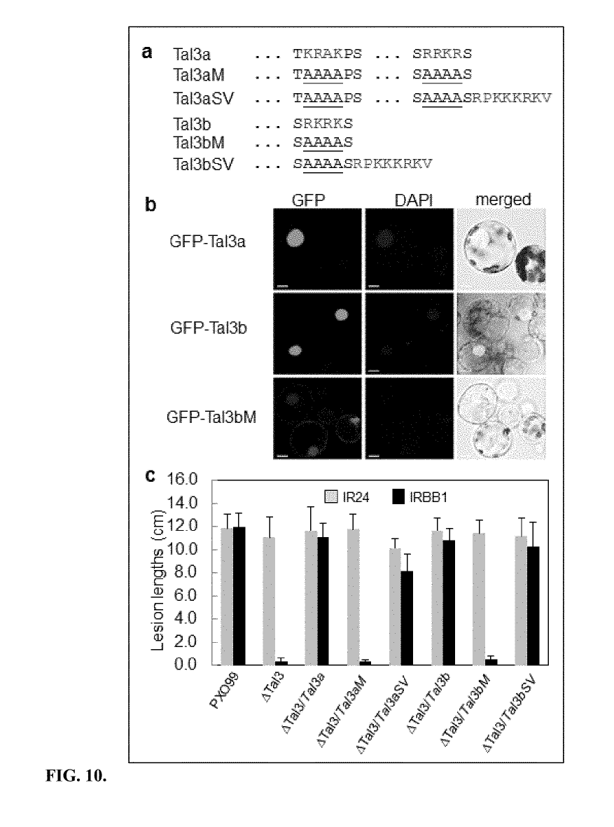

Tal3a was characterized in more detail to determine the requirement of each domain for activity of the iTALEs in Xa1 context. Internal deletions of the central repeats resulted in three Tal3a variants that were expressed at a similar level in bacterial cells (FIG. 6); all except one with 2.5 repeats retained ability to suppress the resistance responses to ΔTal3 in IRBB1 and Xa1 transgenic Kitaake (FIGS. 7A, 7B). Similarly, Tal3a variants swapped with repeat domains from AvrXa7, AvrXa10 and PthXo1 were also able to suppress the resistance responses to ΔTal3 in IRBB1 (FIG. 8). The results suggest the indispensability but not specificity of the repeat domain for suppressive activity of Tal3a. The N-terminus unique in two internal deletions in Tal3a was also tested for its contribution to the suppression. The N-terminus of PthXo1 was swapped with the Tal3a corresponding region, the resultant Tal3a variant containing the N-terminal region of PthXo1 and Tal3a repetitive and C-terminal regions lost the ability to suppress the resistance triggered by ΔTal3 in IRBB1 and Xa1 transgenic plants (FIG. 7C). Similarly, swapping AvrXa7 N-terminus into Tal3a also resulted in the loss of suppressive activity of Tal3a (FIG. 9). Likewise, Tal3a with the full-length C terminal region of PthXo1 due to domain swapping lost its ability to suppress the resistance to ΔTal3 in IRBB1 and Xa1 transgenic plants (FIG. 7C). In their truncated C-termini, Tal3a still retains two nuclear localization signals (NLS) and Tal3b acquires a NLS because of frame shift at its 3′ end (FIG. 3); the NLS motifs were functional in directing the GFP-tagged Tal3a and Tal3b to the nuclei of rice protoplasts (FIGS. 10A, 10B). When tested in plants, Tal3a and Tal3b variants with mutated NLS lost their abilities to suppress the resistance triggered by ΔTal3 in IRBB1; the addition of the SV40 T-antigen NLS restored their activities (FIG. 10C). The results indicate that the unique N- and C-terminal structures of Tal3a and Tal3b are essential for the iTALEs to interfere with the disease resistance controlled by Xa1.

### Xa1 Activates Resistance in Response to Full-Length TALEs

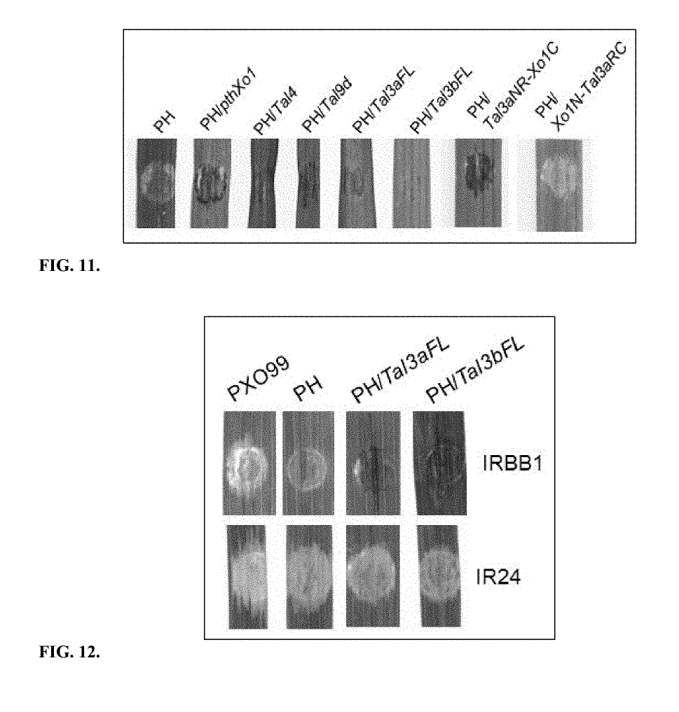

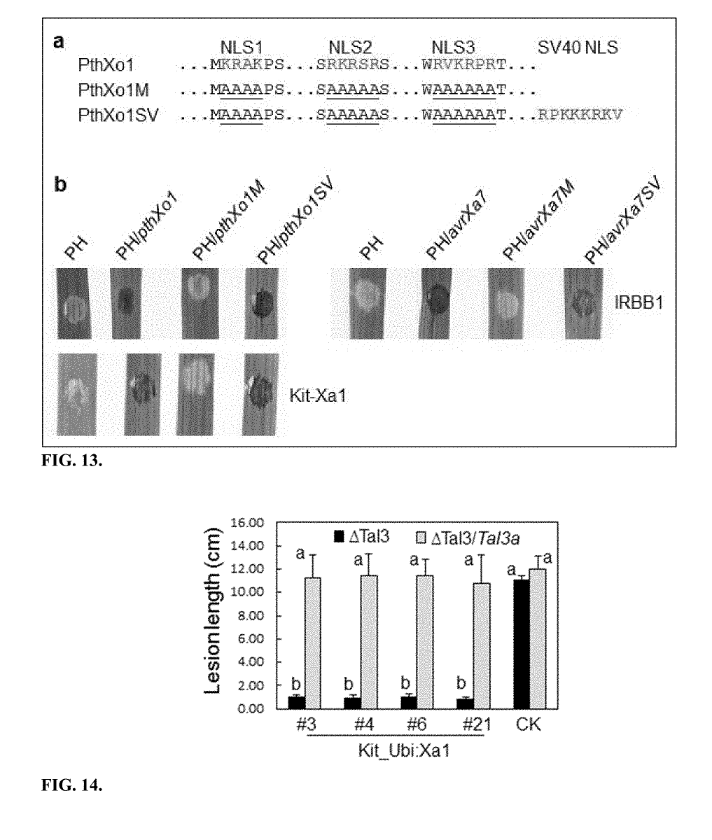

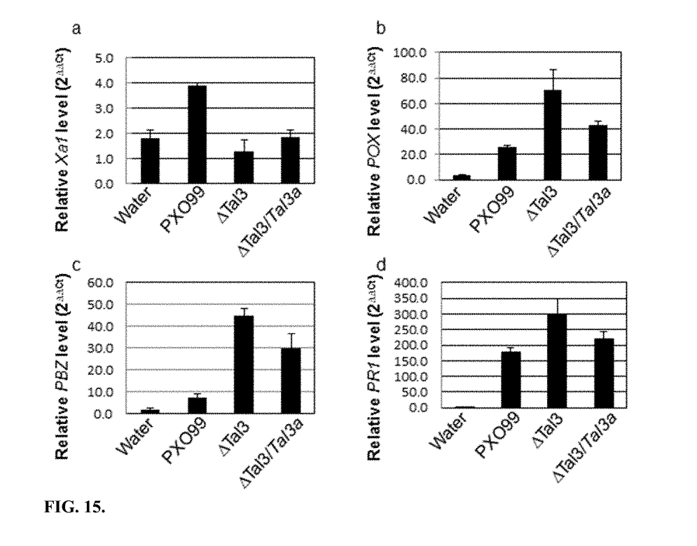

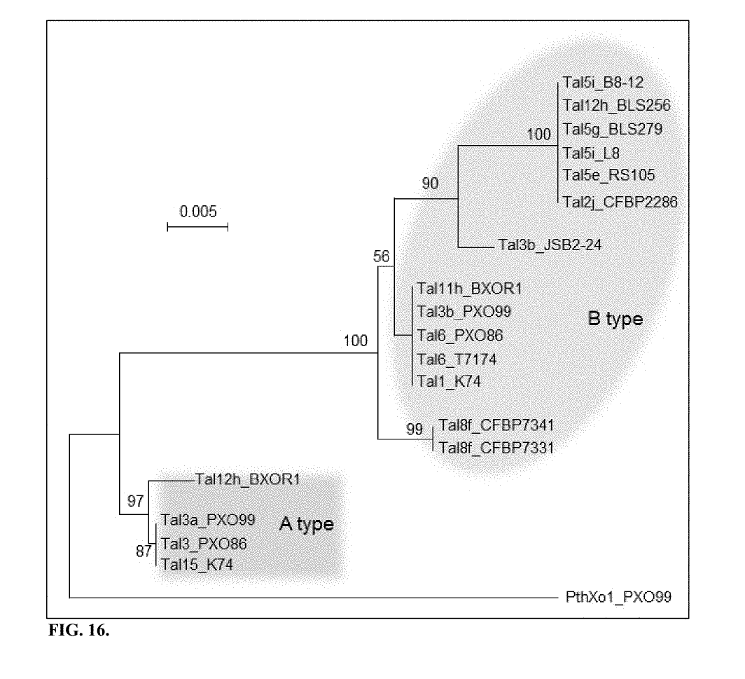

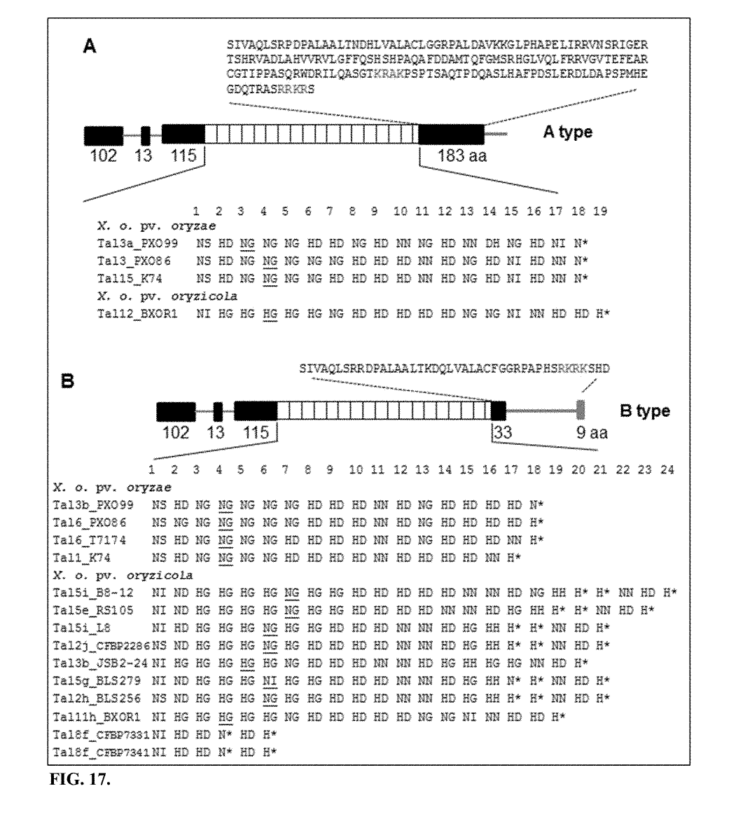

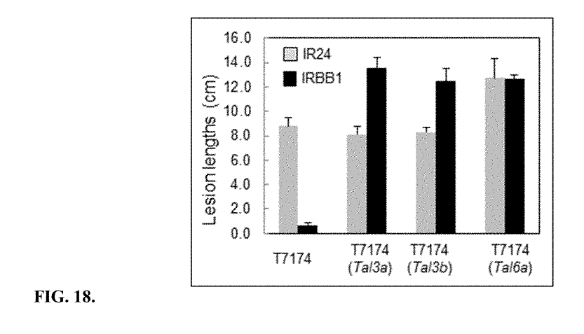

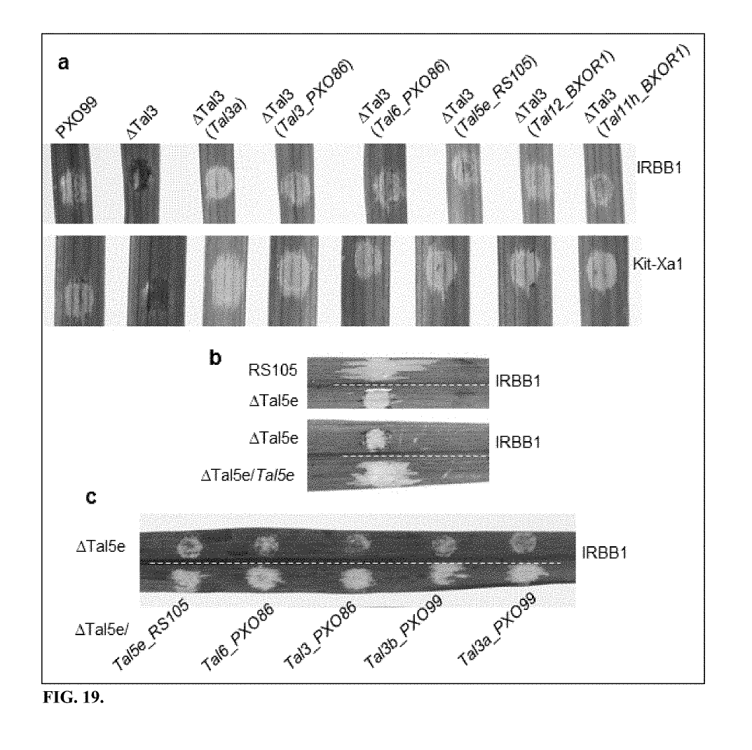

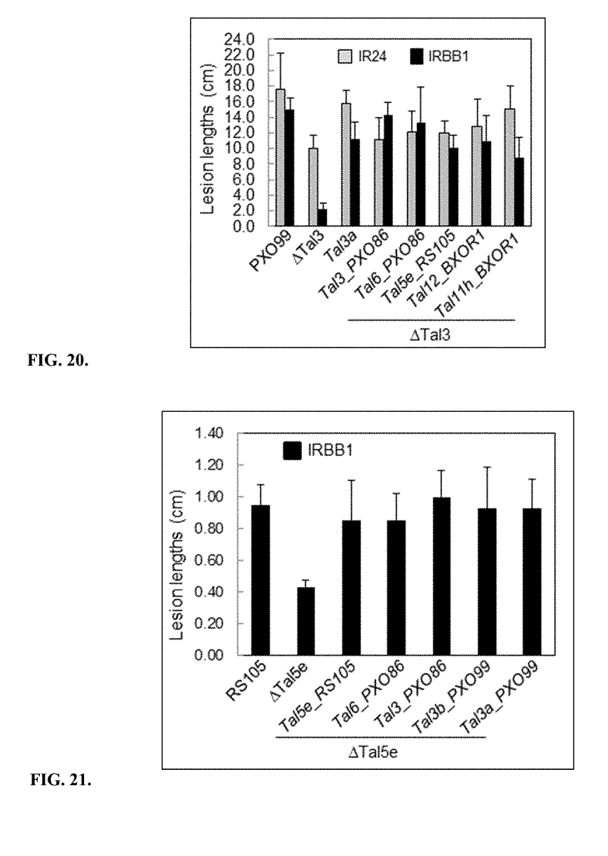

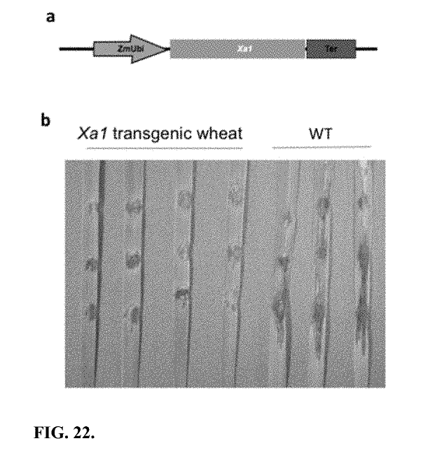

In the initial disease assay with the TALE cluster deletion mutants, the resistance in IRBB1 appeared when the clustered Tal3a and Tal3b were deleted and retained till remaining TALE clusters were deleted. We surmised that Xa1 might recognize TALEs and confer resistance against the pathogen only in absence of iTALE genes. To test the hypothesis, we introduced TALE genes (pthXo1, Tal4 and Tal9d) from PXO99 individually into PH, the TALE-free mutant of PXO99. The resulting TALE-containing strains induced strong HR in Xa1 transgenic Kitaake (FIG. 11). Similarly, Tal3a and Tal3b variants that contain the full-length C-termini due to domain swapping with PthXo1 also triggered HR in Xa1 transgenic plants (FIG. 11) and IRBB1 (FIG. 12). However, PthXo1 and AvrXa7 variants with the NLS mutated lost their abilities to trigger HR in IRBB1 and Xa1 transgenic plants; while addition of SV40 T-antigen NLS restored their activities, suggesting a nuclear site of action of XA1/TALEs (FIG. 13).

iTALE Tal3a Suppresses Xa1 Resistance not Through Interference with Xa1 Expression

To determine whether Xa1, like Xa27 and other “executor” R genes (15, 16, 21, 22), recognizes TALEs through its promoter-specific transcription activation and iTALE overcomes resistance by suppressing Xa1 induction, we made a construct expressing Xa1 coding sequence under the promoter of a rice ubiquitin gene (Os02g06640). The Ubi:Xa1 transgenic Kitaake lines (n=4) were completely resistant to ΔTal3 and the resistance was suppressed in presence of Tal3a (FIG. 14). The results indicate that the mode of action by iTALE is not through interference with Xa1 transcription. To characterize the molecular role of iTALE in suppression of Xa1 resistance in rice, three typical defense genes (peroxidase, PBZ and PR1) that are highly activated particularly during resistance response were checked using the quantitative RT-PCR approach. Xa1 was induced slightly by wounding and bacterial infection, in agreement with the previous study¹⁴. In a contrast, in the incompatible interaction (Xa1/ΔTal3) all three defense genes were highly activated relative to non-infection and compatible interaction (Xa1/PXO99), while Tal3a suppressed the activations (FIG. 15). The results indicate that iTALE overcomes Xa1 resistance partially through suppressing the activation of defense genes.

Functional iTALE Genes are Prevalent Among Xoo and Xoc Isolates

The indiscriminate recognition of TALEs by Xa1 suggests that Xa1 would be the broadest spectrum R gene known to date that is directed at bacterial blight and the only rice-derived R gene to bacterial streak. To assess the resistance spectrum of Xa1, Xa1 transgenic Kitaake plants were inoculated with thirty-six worldwide X. o. pv. oryzae strains. The plants were resistant to only seven field isolates but susceptible to the majority of thirty-six strains. The narrow resistance spectrum of Xa1 is hard to reconcile to the notion that Xa1 recognizes most, if not all, TALE genes and all examined X. o. pv. oryzae strains contain large numbers (15 to 16) of TALE genes (18, 23). In fact, no R gene has ever been found for X. o. pv. oryzicola pathogen, of which strains contain the highest number (e.g., 27 in BLS256) of TALE genes²⁴. It is conceivable to attribute this to the prevalence of iTALE genes in the majority of X. o. pv. oryzae and X. o. pv. oryzicola populations. For example, Tal3a (referred to as type A) and Tal3b (type B) types of iTALE genes exist in all three X. o. pv. oryzae and all nine X. o. pv. oryzicola strains sequenced and well annotated to date (FIG. 16) (18, 23, 24). The known iTALE genes (n=18) are highly conserved at the nucleotide level (>99% identity) and, if expressed, encode effectors that have nearly identical N-termini in both types and nearly identical C-termini in each type. The predicted iTALEs contain distinct central domains (FIG. 17).

We further assessed the prevalence of the two types of iTALE genes among thirty-six X. o. pv. oryzae strains using a PCR approach with type-specific primers. The seven strains incompatible to Xa1 contain either no detectable iTALE gene (3 strains) or only type B iTALE genes (4 strains). The remaining twenty-nine Xa1-compatible strains indeed contain iTALE genes of either only type A (3 strains) or B (6 strains) or of both type A and B (19 strains) (Table 4). The four Xa1-incompatible strains, including strain T7174, that contain B type iTALE genes may either not be expressed or are expressed at a level not adequate to efficiently suppress Xa1-mediated resistance. To investigate this possibility, the iTALE gene Tal3a or Tal3b from PXO99 or the T7174 iTALE gene Tal6 under the LacZ gene promoter were introduced into T7174. Introduction of each plasmid-borne iTALE gene enabled T7174 to overcome the resistance in IRBB1 (FIG. 18). We also cloned Tal3 (type A) and Tal6 (type B) from PXO86 (X. o. pv. oryzae), Tal11h (type B) and Tal12 (type A) from BROX1 and Tal5e from RS105, two X. o. pv. oryzicola strains. All five iTALE genes, when transferred into ΔTal3, were functional in suppression of Xa1-mediated resistance (i.e., in IRBB1 and Xa1-transgenic Kitaake) (FIG. 19A and FIG. 20). Furthermore, for X. o. pv. oryzicola pathogen, when Tal5e, the only iTALE gene in RS105, was inactivated, the mutant was incompatible on IRBB1, and transfer of Tal5e or any of the four iTALE genes from X. o. pv. oryzae enabled Xa1 compatibility to the RS105 mutant (FIG. 19B, 19C and FIG. 21). The results indicate that the type A and type B iTALE genes are evolutionarily conserved and functionally equivalent to contribute strain virulence by interfering with the R gene Xa1-mediated disease resistance against both X. o. pv. oryzae and X. o. pv. oryzicola.

TABLE 4
Xa1-mediated resistance spectrum to X. o. pv. oryzae field isolates.

| Country | | Disease | | |
|---|---|---|---|---|
| of origin | Strain | reactions^{a} | iTALE type A^{b} | iTALE type B^{b} |
| The Philippines | PXO61 | S | + | + |
| | PXO71 | S | + | + |
| | PXO79 | S | + | + |
| | PXO86 | S | + | + |
| | PXO99^{A} | S | + | + |
| | PXO112 | S | − | + |
| | PXO125 | S | + | + |
| Republic of | KXO85 | R | − | − |
| Korea | JW89011 | R | − | + |
| | K202 | S | + | + |
| Japan | T7174 | R | − | + |
| | H75373 | S | + | + |
| Thailand | Xoo2 | S | + | − |
| India | A3842 | S | + | + |
| | A3857 | S | + | + |
| | PbXo7 | S | + | − |
| Indonesia | IXO56 | S | + | + |
| Nepal | NXO 260 | S | + | + |
| Colombia | CIAT1185 | S | + | + |
| China | ZHE 173 | S | + | − |
| | C1 | S | − | + |
| | C3 | S | + | + |
| | C4 | S | + | + |
| | C5 | S | + | + |
| | C6 | S | − | + |
| | C7 | S | + | + |
| | GD1358 | S | + | + |
| | HB17 | S | + | + |
| | HB21 | S | − | + |
| | HLJ72 | S | − | + |
| | JS49-6 | R | − | − |
| | LN57 | S | − | + |
| | NX42 | S | + | + |
| Australia | Aust-2013 | R | − | + |
| | Aust-R3 | R | − | + |
| Cameroon | AXO1947 | R | − | − |

4. Disease reaction is characterized as “S” for susceptibility to bacterial infection when lesion lengths >5 cm and resistance as “R” when lesion lengths <5 cm in Xa1 transgenic Kitaake 12 days after inoculation.
5. “+” and “−” denotes the presence and absence of PCR product with type-specific primers for the two types (A and B) of iTALE genes on genomic DNA from individual strains.

### DISCUSSION

TALE associated host R genes have been previously identified in rice (Xa27, Xa10 and Xa23, xa13, xa25 and xa41), tomato (Bs4), pepper (Bs3, and Bs4C); all of them except one (Bs4) have been found to be involved in transcriptional activation (dominant R gene) or lack thereof (recessive alleles of the otherwise S genes) by the cognate full-length TALEs (14-16, 22, 25-29). Bs4, a constitutively expressed R gene encoding a nucleotide-binding leucine rich repeat protein in tomato, activates resistance including HR in response to the full-length TALE AvrBs4 as well as mutants derived from various truncations of C-terminus and truncations of large portion of central repetitive and C-terminal regions that lack the nuclear localization and transcription activation domains of AvrBs4, suggesting cytoplasmic perception of AvrBs4 by Bs4 in tomato (14, 30). On the other hand, AvrBs4 can also be recognized by an executor R gene, Bs4C, and trigger resistance in pepper. The recognition requires a match between promoter element of Bs4C and central repeats of AvrBs4 for tight expression of Bs4C, entailing a functionality of full-length AvrBs4 (29). In contrast, Xa1, a NBS-LRR type R gene unrelated to Bs4, recognizes all tested TALEs and initiates resistance in rice; the resistance elicitation requires the functional nuclear localization motif of TALEs. Furthermore, truncated TALEs (i.e. iTALEs) as loss-of-function mutants avoid triggering Xa1 resistance and are also as gain-of-function mutants able to suppress Xa1-resistance triggered by full-length TALEs, analogous to the dominant, negative regulators in host innate immunity.

Rice, evolutionarily speaking, has appeared to hit the jackpot in the acquisition of an R gene that recognizes all or most TAL effectors. From the pathogen stand point, exposure of multiple TALE targets to a cognate host R gene would be conundrum in that at least one TALE is critical for virulence in all strains. We show that two pathogens have evolved a potent adaptation to counteract the Xa1-controlled disease resistance in rice triggered by the large number of TALE genes of two pathogens using the very same genetic components. Understanding how one or two iTALEs efficiently mask the host immunity derived from recognition of multiple targets may enable engineering of more effective R genes that, for example, are less sensitive to the iTALE genes. Xa1 and derived R genes may be an efficient genetic source to combat several other important crop diseases (e.g., citrus canker, wheat blight) wherein the causative Xanthomonas agents possess TALE genes but not iTALE genes. In a broader light, the results reveal that there are lots of annotated and neglected “pseudogenes”, and the seemingly “pseudogenes” in a variety of bacterial genomes may warrant further examination.

### REFERENCES CITED

1. Jones, J. D. & Dangl, J. L. The plant immune system. Nature 444, 323-329 (2006).
2. Feng, F. & Zhou, J. Plant-bacterial pathogen interactions mediated by type III effectors. Curr. Opin. Plant Biol. 15, 469-476 (2012).
3. Flor, H. H. Current status of gene-for-gene concept. Annu. Rev. Phytopathol. 9, 275-296 (1971).
4. Jackson, R. W. et al. Identification of a pathogenicity island, which contains genes for virulence and avirulence, on a large native plasmid in the bean pathogen Pseudomonas syringae pathovar phaseolicola. Proc. Natl. Acad. Sci. U.S.A. 96, 10875-10880 (1999).
5. Tsiamis, G. et al. Cultivar-specific avirulence and virulence functions assigned to avrPphF in Pseudomonas syringae pv. phaseolicola, the cause of bean halo-blight disease. EMBO J. 19, 3204-3214 (2000).
6. White, F. F., Potnis, N., Jones, J. B. & Koebnik, R. The type III effectors of Xanthomonas. Mol. Plant. Pathol. 10, 749-766 (2009).
7. Boch, J. & Bonas, U. Xanthomonas AvrBs3 family-type III effectors: discovery and function. Annu. Rev. Phytopathol. 48, 419-436 (2010).
8. Bogdanove, A. J. & Voytas, D. F. TAL effectors: customizable proteins for DNA targeting. Science 333, 1843-1846 (2011).
9. White, F. F. & Yang, B. Host and pathogen factors controlling the rice-Xanthomonas oryzae interaction. Plant Physiol. 150, 1677-1686 (2009).
10. Cernadas, R. A. et al. Code-assisted discovery of TAL effector targets in bacterial leaf streak of rice reveals contrast with bacterial blight and a novel susceptibility gene. PLoS Pathog. 10, e1003972 (2014).
11. Doyle, E. L., Stoddard, B. L., Voytas, D. F. & Bogdanove, A. J. TAL effectors: highly adaptable phytobacterial virulence factors and readily engineered DNA-targeting proteins. Trends Cell Biol. 23, 390-398 (2013).
12. Boch, J., Bonas, U. & Lahaye, T. TAL effectors-pathogen strategies and plant resistance engineering. New Phytol. 204, 823-832 (2014).
13. Zhang, J., Yin, Z. & White, F. TAL effectors and the executor R genes. Front. Plant. Sci. 6, 641 (2015).
14. Schornack, S. et al. The tomato resistance protein Bs4 is a predicted non-nuclear TIR-NB-LRR protein that mediates defense responses to severely truncated derivatives of AvrBs4 and overexpressed AvrBs3. Plant J. 37, 46-60 (2004).
15. Tian, D. et al. The rice TAL effector-dependent resistance protein XA10 triggers cell death and calcium depletion in the endoplasmic reticulum. Plant Cell 26, 497-515 (2014).
16. Gu, K. et al. R gene expression induced by a type-III effector triggers disease resistance in rice. Nature 435, 1122-1125 (2005).
17. Yang, B., Sugio, A. & White, F. F. Os8N3 is a host disease-susceptibility gene for bacterial blight of rice. Proc. Natl. Acad. Sci. U.S.A. 103, 10503-10508 (2006).
18. Salzberg, S. L. et al. Genome sequence and rapid evolution of the rice pathogen Xanthomonas oryzae pv. oryzae PXO99A. BMC Genomics 9, 204 (2008).
19. Ogawa, T., Yamamoto, T., Khush, G. S., Mew, T. W. & Kaku, H. Near-isogenic lines as international differentials for resistance to bacterial blight of rice. Rice Genetics Newsletter 5, 106-107 (1988).
20. Yoshimura, S. et al. Expression of Xa1, a bacterial blight-resistance gene in rice, is induced by bacterial inoculation. Proc. Natl. Acad. Sci. U.S.A. 95, 1663-1668 (1998).
21. Romer, P. et al. Plant pathogen recognition mediated by promoter activation of the pepper Bs3 resistance gene. Science 318, 645-648 (2007).
22. Wang, C. et al. XA23 is an executor R protein and confers broad-spectrum disease resistance in rice. Mol. Plant. 8, 290-302 (2015).
23. Ochiai, H., Inoue, Y., Takeya, M., Sasaki, A. & Kaku, H. Genome sequence of Xanthomonas oryzae pv. Oryzae suggests contribution of large numbers of effector genes and insertion sequences to its race diversity. Jpn. Agric. Res. Quart. 39, 275-187 (2005).
24. Wilkins, K. E., Booher, N. J., Wang, L. & Bogdanove, A. J. TAL effectors and activation of predicted host targets distinguish Asian from African strains of the rice pathogen Xanthomonas oryzae pv. oryzicola while strict conservation suggests universal importance of five TAL effectors. Front. Plant. Sci. 6, 536 (2015).
25. Chu, Z. et al. Targeting xa13, a recessive gene for bacterial blight resistance in rice. Theor. Appl. Genet. 112, 455-461 (2006).
26. Liu, Q. et al. A paralog of the MtN3/saliva family recessively confers race-specific resistance to Xanthomonas oryzae in rice. Plant. Cell. Environ. 34, 1958-1969 (2011).
27. Hutin, M., Sabot, F., Ghesquiere, A., Koebnik, R. & Szurek, B. A knowledge-based molecular screen uncovers a broad-spectrum OsSWEET14 resistance allele to bacterial blight from wild rice. Plant J. 84, 694-703 (2015).
28. Romer, P. et al. Recognition of AvrBs3-like proteins is mediated by specific binding to promoters of matching pepper Bs3 alleles. Plant Physiol. 150, 1697-1712 (2009).
29. Strauss, T. et al. RNA-seq pinpoints a Xanthomonas TAL-effector activated resistance gene in a large-crop genome. Proc. Natl. Acad. Sci. U.S.A. 109, 19480-19485 (2012).
30. Ballvora, A. et al. Genetic mapping and functional analysis of the tomato Bs4 locus governing recognition of the Xanthomonas campestris pv. vesicatoria AvrBs4 protein. Mol. Plant Microbe Interact. 14, 629-638 (2001).
31. L. Zou, Y. Li, G. Chen, A non-marker mutagenesis strategy to generate poly-hrp Gene mutants in the rice pathogen Xanthomonas oryzae pv. oryzicola. Agricultural Sciences in China. 10, 1139-1150 (2011).
32. Ausubel, F., Brent, R., Kingston, R., Moore, D., Seidman, J., Struhl, K. in Current Protocols in Molecular Biology (Canada John Wiley and Sons, New York, 1993).
33. Yang, B. & White, F. F. Diverse members of the AvrBs3/PthA family of type III effectors are major virulence determinants in bacterial blight disease of rice. Mol. Plant Microbe Interact. 17, 1192-1200 (2004).
34. Zhang, Y. et al. A highly efficient rice green tissue protoplast system for transient gene expression and studying light/chloroplast-related processes. Plant. Methods 7, 30-4811-7-30 (2011).
35. Livak, K. J. & Schmittgen, T. D. Analysis of relative gene expression data using real-time quantitative PCR and the 2(-Delta Delta C(T)) Method. Methods 25, 402-408 (2001).
36. Hiei, Y., Ohta, S., Komari, T. & Kumashiro, T. Efficient transformation of rice (Oryza sativa L.) mediated by Agrobacterium and sequence analysis of the boundaries of the T-DNA. Plant J. 6, 271-282 (1994).
37. Yang, B. & Bogdanove, A. Inoculation and virulence assay for bacterial blight and bacterial leaf streak of rice. Methods Mol. Biol. 956, 249-255 (2013).

### Example 2

### Xa1 Transgenic Wheat Plants Confer Resistance to Wheat Bacterial Blight

As the most widely cultivated crop with the highest trading value, wheat (Triticum aestivum L.) provides about 20% of our daily calories and protein supply (FOA Stat. 2015, http://www.fao.org/faostat/en/#home). Like other corps, wheat also suffers yield losses due to biotic and abiotic stresses. The biotic stresses include fungal diseases such as leaf rust, bacterial diseases and insect pests. Bacterial blight of wheat, caused by Xanthomonas translucens is one of severe wheat bacterial diseases. The disease is also called bacterial leaf streak when occurring on leaves or black chaff when on the glumes. Yield losses to the blight could range as high as 40% in the most severely infected fields to generally 10% or less (e.g., in Idaho) (Forster et al. 1986). If severely inflicted, the wheat spikes may be sterile (Forster and Shaad 1988). Currently there is no effective control measures for wheat blight. Genetic resistance seems to be most effective, cost saving and environmentally friendly control measures. Unfortunately, there is no blight resistance gene been identified and molecularly cloned so far.

Wheat blight pathogen Xanthomonas translucens also contain multiple TAL effector genes though their role in pathogenesis of wheat blight is poorly known (Peng et al. 2016). It is possible that rice Xa1 when expressed in wheat can confer resistance to TAL effector-containing X. translucens strains, reducing disease symptom, for example water-soaking at the inoculation site similarly to bacterial blight of rice. To test such possibility, we generated transgenic wheat with Xa1 under the maize ubiquitin gene 1 promoter, and assessed the disease symptoms infected with X. translucens.

FIG. 22a illustrates the gene construct used for wheat transformation. Twenty-one independent transgenic wheat lines have been obtained through biolistic bombardment gene delivery system into wheat cultivar Bobwhite. Two lines that were confirmed to contain the transgene Xa1 were used to do bacterial infection through syringe infiltration with bacterial inoculum (OD600=0.15). Infiltrated plant leaves were measured for water-soaking at the inoculated sites 4 days post inoculation. The results clearly showed the less severe symptom in transgenic plants than wild type Bobwhite.

Transgenic wheat (T1 plants) containing rice disease resistance gene Xa1 confers resistance to wheat bacterial blight, caused by Xanthomonas translucens. Transgenic seedlings (20 days old) were infiltrated with bacterial inoculum and photographed 4 days after inoculation. Please note the water soaking spots were confined at the inoculation spots in transgenic plants while in wild type plant water soaking spread far beyond the inoculation spots.

### REFERENCES

Forster, R. L., Mihuta-Grimm, L., and Schaad, N. W. 1986. Black chaff of wheat and barley. University of Idaho, College of Agriculture. Current Information Series No. 784, p. 2. Forster, R. L., and Schaad, N. W. 1988. Control of black chaff of wheat with seed treatment and a foundation seed health program. Plant Disease 72:935-938.
Zhao Peng, Ying Hu, Jingzhong Xie, Neha Potnis, Alina Akhunova, Jeffrey Jones, Zhaohui Liu, Frank F. White, and Sanzhen Liu. 2016. Long read and single molecule DNA sequencing simplifies genome assembly and TAL effector gene analysis of Xanthomonas translucens. BMC Genomics 17:21.

TABLE OF SEQUENCES

| | | |
|---|---|---|
| SEQ ID NO: 1 | Nucleotide | Xa1 |
| SEQ ID NO: 2 | Protein | Xa1 |
| SEQ ID NO: 3 | Nucleotide | Xa2 |
| SEQ ID NO: 4 | Protein | Xa2 |
| SEQ ID NO: 5 | Nucleotide | iTAL3a |
| SEQ ID NO: 6 | Protein | iTAL3a |
| SEQ ID NO: 7 | Nucleotide | iTAL3b |
| SEQ ID NO: 8 | Protein | iTAL3b |

Xa1 genomic sequence SEQ ID NO: 1
ATGGAGGAGGTGGAAGCCGGTTGGCTGGAGGGCGGGATCAGGTGGCTGGCGGAGACCATCCTGGATAACCTGGAC
GCCGACAAGCTGGATGAATGGATTCGCCAGATTAGGCTCGCCGCTGACACCGAGAAGCTACGGGCTGAGATCGAG
AAGGTGGATGGGGTGGTGGCTGCCGTGAAGGGGAGGGCGATCGGGAACAGGTCGCTGGCCCGATCGCTCGGCCGT
CTCAGGGGGTTGCTGTACGACGCCGACGATGCGGTCGACGAGCTCGACTACTTCAGGCTCCAGCAGCAGGTCGAG
GGAGGAGTTACTACACGGTTTGAGGCTGAAGAGACGGTCGGAGATGGAGCAGAGGACGAGGACGATATTCCGATG
GACAATACTGATGTACCGGAGGCAGTGGCGGCAGGCAGCAGCAAGAAACGGTCCAAGGCATGGGAACACTTTACT
ACCGTAGAGTTCACTGCTGACGGGAAGGATTCTAAAGCACGGTGCAAGTACTGCCACAAGGACCTATGTTGCACA
TCTAAGAACGGGACATCAGCTTTGCGCAACCATCTCAATGTTTGCAAGAGGAAACGTGTAACAAGTACTGACCAA
CCGGTAAATCCATCAAGTGCCGGTGAGGGTGCATCAAATGCAACTGGTAATTCAGTTGGCAGAAAAAGGATGAGA
ATGGATGGGACTTCAACACACCACGAGGCAGTTAGCACGCACCCTTGGAACAAGGCTGAACTTTCCAACAGGATC
CAATGCATGACTCATCAGTTAGAAGAGGCTGTAAATGAGGTTATGAGGCTATGTCGATCCTCAAGTTCAAACCAG
AGTCGACAGGGTACACCACCGGCCACAAATGCAACAACATCGTCTTATCTTCCGGAGCCCATAGTGTATGGGAGG
GCTGCAGAGATGGAAACCATCAAACAGCTGATCATGAGCAATAGATCTAATGGCATAACCGTCCTGCCAATTGTA
GGCAATGGAGGGATAGGAAAAACCACTTTGGCGCAACTGGTCTGCAAAGATCTGGTAATTAAAAGTCAGTTTAAT
GTTAAGATATGGGTGTATGTATCTGATAAATTTGATGTAGTTAAGATTACAAGGCAGATTTTGGATCATGTCTCC
AACCAGAGCCACGAAGGAATAAGCAACCTTGATACGCTTCAGCAGGATCTTGAGGAACAAATGAAATCTAAGAAG
TTCCTCATTGTCTTAGATGATGTGTGGGAAATCCGTACAGATGACTGGAAAAAACTACTGGCTCCTTTAAGACCT
AATGATCAGGTGAATTCATCACAGGAAGAGGCAACAGGTAATATGATAATTTTGACAACTCGTATACAGAGTATT
GCCAAAAGTCTTGGAACAGTACAATCAATTAAGTTAGAAGCTCTGAAAGATGACGATATATGGTCACTATTTAAA
GTGCATGCTTTTGGTAATGATAAACATGATAGTAGTCCAGGCTTACAGGTTCTTGGGAAGCAAATTGCTAGCGAG
CTAAAAGGCAACCCACTGGCAGCAAAAACTGTGGGTTCACTATTAGGAACGAATCTTACCATCGATCATTGGGAT
AGCATTATAAAGAGTGAAGAATGGAAATCCCTGCAACAAGCTTATGGCATCATGCAAGCGCTGAAGTTGAGCTAT
GATCATCTATCCAACCCCTTACAGCAATGCGTCTCTTATTGTTCTCTTTTCCCCAAGGGTTATTCTTTCAGCAAA
GCACAACTAATACAAATATGGATTGCTCAAGGATTTGTGGAAGAATCCAGTGAGAAGTTGGAGCAGAAAGGATGG
AAATATCTAGCTGAGTTGGTAAATTCGGGTTTCCTTCAGCAAGTTGAAAGCACACGGTTTTCATCAGAATATTTT
GTTATGCACGATCTTATGCATGATTTAGCGCAAAAGGTTTCACAAACAGAATATGCAACTATAGATGGCTCAGAG
TGCACAGAGTTAGCCCCAAGTATACGCCATTTGTCAATAGTAACTGATTCTGCATACCGCAAGGAGAAATATAGA
AACATATCTCGTAATGAGGTGTTTGAGAAAAGGTTGATGAAAGTTAAGTCAAGGAGTAAGTTGAGGTCACTGGTA
TTAATTGGGCAATATGATTCTCATTTTTTTAAATATTTCAAAGATGCTTTCAAGGAAGCACAACATCTGCGACTG
CTGCAGATCACTGCAACTTATGCTGATTCTGATTCATTTCTCTCCAGTTTGGTAAATTCTACACATCTCCGGTAT
CTGAAAATTGTGACCGAAGAATCCGGCAGAACTTTGCCCCGATCTCTAAGGAAGTATTACCATCTTCAAGTACTA
GATATTGGCTATAGATTTGGAATTCCCCGTATATCTAATGATATAAATAATCTTCTCAGCCTGCGGCATCTTGTT
GCATATGATGAAGTGTGTTCTTCCATTGCTAACATTGGTAAAATGACCTCACTTCAGGAACTAGGCAATTTTATT
GTTCAGAATAATTTAAGTGGTTTTGAGGTGACACAATTGAAATCCATGAACAAGCTTGTACAACTTAGTGTGTCT
CAGCTTGAAAATGTTAGAACTCAGGAGGAGGCATGTGGGGCAAAACTGAAAGACAAACAACACTTAGAAAAGCTA
CATTTGTCCTGGAAGGATGCATGGAATGGATATGACAGTGACGAAAGCTATGAAGATGAATACGGCAGTGATATG
AATATAGAAACAGAAGGGGAGGAACTGTCAGTTGGTGATGCCAATGGTGCCCAAAGCTTACAACATCACAGTAAT
ATAAGCTCTGAACTTGCTTCAAGTGAGGTGCTCGAAGGTCTTGAACCACATCACGGCCTCAAGTATCTACGGATA
TCTGGGTATAATGGATCTACCTCCCCAACTTGGCTTCCTTCTTCACTTACCTGTCTGCAAACACTTCATCTAGAA
AAATGTGGAAAATGGCAAATACTTCCTTTAGAAAGGCTAGGGTTACTTGTAAAGCTCGTGTTGATCAAAATGAGG
AATGCAACAGAACTCTCAATCCCTTCACTGGAGGAGCTTGTGTTAATTGCATTGCCAAGCTTGAACACATGCTCC
TGCACTTCCATCAGGAACTTGAACTCCAGTTTAAAGGTTCTGAAAATTAAGAATTGCCCTGTACTGAAGGTATTT
CCCTTGTTTGAGATTTCCCAGAAATTTGAAATCGAGCGGACGTCGTCATGGTTGCCCCATCTTAGCAAGCTTACC
ATCTATAATTGTCCTCTTTCCTGTGTGCACAGTTCTCTGCCACCTTCCGCAATCAGTGGTTATGGAGAATATGGA
AGGTGTACCCTTCCGCAATCACTTGAGGAACTTTACATCCATGAGTATTCTCAAGAAACTCTGCAGCCCTGCTTT
TCAGGGAACCTCACTCTCCTGAGAAAATTACATGTACTGGGAAACTCAAATTTAGTGTCTCTGCAGCTCCATTCC
TGCACAGCACTCGAAGAGTTGATAATTCAAAGCTGTGAGTCTCTTAGTTCTCTGGATGGCTTGCAATTGCTTGGC
AATCTCAGGTTGCTGCGGGCACATAGATGCCTCAGTGGTCATGGAGAAGATGGAAGGTGTATCCTTCCGCAATCA
CTTGAGGAACTTTACATCCATGAGTATTCTCAAGAAACTCTGCAGCCCTGCTTTTCAGGGAACCTCACTCTCCTG
AGAAAATTACATGTACTGGGAAACTCAAATTTAGTGTCTCTGCAGCTCCATTCCTGCACAGCACTCGAAGAGTTG
ATAATTCAAAGCTGTGAGTCTCTTAGTTCTCTGGATGGCTTGCAATTGCTTGGCAATCTCAGGTTGCTGCGGGCA
CATAGATGCCTCAGTGGTCATGGAGAAGATGGAAGGTGTATCCTTCCGCAATCACTTGAGGAACTTTACATCCAT
GAGTATTCTCAAGAAACTCTGCAGCCCTGCTTTTCAGGGAACCTCACTCTCCTGAGAAAATTACATGTACTGGGA
AACTCAAATTTCGTGTCTCTGCAGCTCCATTCCTGCACAGCACTCGAAGAGTTGATAATTCAAAGCTGTGAGTCT
CTTAGTTCTCTGGATGGCTTGCAATTGCTTGGCAATCTCAGGTTGCTGCAGGCACATAGATGCCTCAGTGGTCAT
GGAGAAGATGGAAGGTGTATCCTTCCGCAATCACTTGAGGAACTTTACATCCATGAGTATTCTCAAGAAACTCTG
CAGCCCTGCTTTTCAGGGAACCTCACTCTCCTGAGAAAATTACATGTACTTGGAAACTCAAATTTAGTGTCTCTG
CAGCTCCATTCCTGCACAGCACTCGAAGAGTTGATAATTCAAAGCTGTGAGTCTCTTAGTTCTCTGGATGGCTTG
CAATTGCTTGGCAATCTCAGGTTGCTGCAGGCACATAGATGCCTCAGTGGTCATGGAGAAGATGGAAGGTGTATC
CTTCCGCAATCACTTGAGGAACTTTACATCCATGAGTATTCTCAAGAAACTCTGCAGCCCTGCTTTTCAGGGAAC
CTCACTCTCCTGAGAAAATTACATGTACTGGGAAACTCAAATTTAGTGTCTCTGCAGCTCCATTCCTGCACATCA
CTCGAAGAGTTGAAAATTCAAAGCTGTGAGTCTCTTAGTTCTCTGGATGGCTTGCAATTGCTTGGCAATCTCAGG
TTGCTGCAGGCACATAGATGCCTCAGTGGTCATGGAGAAGATGGAAGGTGTATCCTTCCGCAATCACTTGAGGAA
CTTTTCATCAGTGAGTATTCTCTAGAAACTCTGCAGCCCTGCTTCCTGACGAATCTCACCTGCTTAAAACAATTA
GAGGTATCAGGCACCACAAGTTTAAAATCTCTAGAACTGCAATCATGCACTGCACTCGAACATTTGAAGATTCAA
GGTTGTGCGTCGCTTGCTACATTGGAGGGGTTGCAATTCCTCCACGCCCTCAGGCATATGAAAGTATTCAGATGC
CCTGGCTTGCCTCCATATTTGGGGAGTTCGTCAGAGCAGGGCTATGAGCTATGCCCACGACTGGAAAGGCTCGAC
ATCGATGACCCCTCTATCCTTACCACGTCGTTCTGCAAGCACCTCACCTCCCTCCAACGCCTAGAGCTTAACTAT
TGCGGAAGTGAAGTGGCAAGACTAACGGATGAGCAAGAGAGAGCGCTTCAGCTCCTCACGTCCCTGCAAGAGCTC
CGGTTTAAGTATTGCTACAATCTCATAGATCTTCCTGCGGGGCTCCACAGCCTTCCCTCCCTCAAGAGGTTGGAG
ATCCGGAGTTGCAGGAGCATCGCGAGGCTGCCGGAGAAGGGCCTCCCACCTTCGTTCGAAGAACTGGATATCATC
GCTTGCAGTAATGAGCTAGCTCAGCAGTGCAGAACTCTAGCAAGCACTCTGAAGGTCAAAATTAATGGGGGATAT
GTGAACTGA
Xa1 protein SEQ ID NO: 2
MEEVEAGWLEGGIRWLAETILDNLDADKLDEWIRQIRLAADTEKLRAEIEKVDGVVAAVKGRAIGNRSLARSLGR
LRGLLYDADDAVDELDYFRLQQQVEGGVTTRFEAEETVGDGAEDEDDIPMDNTDVPEAVAAGSSKKRSKAWEHFT
TVEFTADGKDSKARCKYCHKDLCCTSKNGTSALRNHLNVCKRKRVTSTDQPVNPSSAGEGASNATGNSVGRKRMR
MDGTSTHHEAVSTHPWNKAELSNRIQCMTHQLEEAVNEVMRLCRSSSSNQSRQGTPPATNATTSSYLPEPIVYGR
AAEMETIKQLIMSNRSNGITVLPIVGNGGIGKTTLAQLVCKDLVIKSQFNVKIWVYVSDKEDVVKITRQILDHVS
NQSHEGISNLDTLQQDLEEQMKSKKFLIVLDDVWEIRTDDWKKLLAPLRPNDQVNSSQEEATGNMIILTTRIQSI
AKSLGTVQSIKLEALKDDDIWSLFKVHAFGNDKHDSSPGLQVLGKQIASELKGNPLAAKTVGSLLGTNLTIDHWD
SIIKSEEWKSLQQAYGIMQALKLSYDHLSNPLQQCVSYCSLFPKGYSFSKAQLIQIWIAQGFVEESSEKLEQKGW
KYLAELVNSGFLQQVESTRFSSEYFVMHDLMHDLAQKVSQTEYATIDGSECTELAPSIRHLSIVTDSAYRKEKYR
NISRNEVFEKRLMKVKSRSKLRSLVLIGQYDSHFFKYFKDAFKEAQHLRLLQITATYADSDSFLSSLVNSTHLRY
LKIVTEESGRTLPRSLRKYYHLQVLDIGYREGIPRISNDINNLLSLRHLVAYDEVCSSTANIGKMTSLQELGNFI
VQNNLSGFEVTQLKSMNKLVQLSVSQLENVRTQEEACGAKLKDKQHLEKLHLSWKDAWNGYDSDESYEDEYGSDM
NIETEGEELSVGDANGAQSLQHHSNISSELASSEVLEGLEPHHGLKYLRISGYNGSTSPTWLPSSLTCLQTLHLE
KCGKWQILPLERLGLLVKLVLIKMRNATELSIPSLEELVLIALPSLNTCSCTSIRNLNSSLKVLKIKNCPVLKVF
PLFEISQKFEIERTSSWLPHLSKLTIYNCPLSCVHSSLPPSAISGYGEYGRCTLPQSLEELYIHEYSQETLQPCF
SGNLTLLRKLHVLGNSNLVSLQLHSCTALEELIIQSCESLSSLDGLQLLGNLRLLRAHRCLSGHGEDGRCILPQS
LEELYIHEYSQETLQPCFSGNLTLLRKLHVLGNSNLVSLQLHSCTALEELIIQSCESLSSLDGLQLLGNLRLLRA
HRCLSGHGEDGRCILPQSLEELYIHEYSQETLQPCFSGNLTLLRKLHVLGNSNFVSLQLHSCTALEELIIQSCES
LSSLDGLQLLGNLRLLQAHRCLSGHGEDGRCILPQSLEELYIHEYSQETLQPCFSGNLTLLRKLHVLGNSNLVSL
QLHSCTALEELIIQSCESLSSLDGLQLLGNLRLLQAHRCLSGHGEDGRCILPQSLEELYIHEYSQETLQPCFSGN
LTLLRKLHVLGNSNLVSLQLHSCTSLEELKIQSCESLSSLDGLQLLGNLRLLQAHRCLSGHGEDGRCILPQSLEE
LEISEYSLETLQPCFLTNLTCLKQLEVSGTTSLKSLELQSCTALEHLKIQGCASLATLEGLQFLHALRHMKVERC
PGLPPYLGSSSEQGYELCPRLERLDIDDPSILTTSFCKHLTSLQRLELNYCGSEVARLTDEQERALQLLTSLQEL
RFKYCYNLIDLPAGLHSLPSLKRLEIRSCRSIARLPEKGLPPSFEELDIIACSNELAQQCRTLASTLKVKINGGY
VN
Xa2 Genomic Sequence SEQ ID NO: 3
ATGGAGGAGGTGGAAGCCGGTTGGCTGGAGGGCGGGATCAGGTGGCTGGCGGAGACCATCCTGGATAACCTGGAC
GCCGACAAGCTGGATGAATGGATTCGCCAGATTAGGCTCGCCGCTGACACCGAGAAGCTACGGGCTGAGATCGAG
AAGGTGGATGGGGTGGTGGCTGCCGTGAAGGGGAGGGCGATCGGGAACAGGTCGCTGGCCCGATCGCTCGGCCGT
CTCAGGGGGTTGCTGTACGACGCCGACGATGCGGTCGACGAGCTCGACTACTTCAGGCTCCAGCAGCAGGTCGAG
GGAGGAGTTACTACACGGTTTGAGGCTGAAGAGACGGTCGGAGATGGAGCAGAGGACGAGGACGATATTCCGATG
GACAATACTGATGTACCGGAGGCAGTGGCGGCAGGCAGCAGCAAGAAACGGTCCAAGGCATGGGAACACTTTACT
ACCGTAGAGTTCACTGCTGACGGGAAGGATTCTAAAGCACGGTGCAAGTACTGCCACAAGGACCTATGTTGCACA
TCTAAGAACGGGACATCAGCTTTGCGCAACCATCTCAATGTTTGCAAGAGGAAACGTGTAACAAGTACTGACCAA
CCGGTAAATCCATCAAGTGCCGGTGAGGGTGCATCAAATGCAACTGGTAATTCAGTTGGCAGAAAAAGGATGAGA
ATGGATGGGACTTCAACACACCACGAGGCAGTTAGCACGCACCCTTGGAACAAGGCTGAACTTTCCAACAGGATC
CAATGCATGACTCATCAGTTAGAAGAGGCTGTAAATGAGGTTATGAGGCTATGTCGATCCTCAAGTTCAAACCAG
AGTCGACAGGGTACACCACCGGCCACAAATGCAACAACATCGTCTTATCTTCCGGAGCCCATAGTGTATGGGAGG
GCTGCAGAGATGGAAACCATCAAACAGCTGATCATGAGCAATAGATCTAATGGCATAACCGTCCTGCCAATTGTA
GGCAATGGAGGGATAGGAAAAACCACTTTGGCGCAACTGGTCTGCAAAGATCTGGTAATTAAAAGTCAGTTTAAT
GTTAAGATATGGGTGTATGTATCTGATAAATTTGATGTAGTTAAGATTACAAGGCAGATTTTGGATCATGTCTCC
AACCAGAGCCACGAAGGAATAAGCAACCTTGATACGCTTCAGCAGGATCTTGAGGAACAAATGAAATCTAAGAAG
TTCCTCATTGTCTTAGATGATGTGTGGGAAATCCGTACAGATGACTGGAAAAAACTACTGGCTCCTTTAAGACCT
AATGATCAGGTGAATTCATCACAGGAAGAGGCAACAGGTAATATGATAATTTTGACAACTCGTATACAGAGTATT
GCCAAAAGTCTTGGAACAGTACAATCAATTAAGTTAGAAGCTCTGAAAGATGACGATATATGGTCACTATTTAAA
GTGCATGCTTTTGGTAATGATAAACATGATAGTAGTCCAGGCTTACAGGTTCTTGGGAAGCAAATTGCTAGCGAG
CTAAAAGGCAACCCACTGGCAGCAAAAACTGTGGGTTCACTATTAGGAACGAATCTTACCATCGATCATTGGGAT
AGCATTATAAAGAGTGAAGAATGGAAATCCCTGCAACAAGCTTATGGCATCATGCAAGCGCTGAAGTTGAGCTAT
GATCATCTATCCAACCCCTTACAGCAATGCGTCTCTTATTGTTCTCTTTTCCCCAAGGGTTATTCTTTCAGCAAA
GCACAACTAATACAAATATGGATTGCTCAAGGATTTGTGGAAGAATCCAGTGAGAAGTTGGAGCAGAAAGGATGG
AAATATCTAGCTGAGTTGGTAAATTCGGGTTTCCTTCAGCAAGTTGAAAGCACACGGTTTTCATCAGAATATTTT
GTTATGCACGATCTTATGCATGATTTAGCGCAAAAGGTTTCACAAACAGAATATGCAACTATAGATGGCTCAGAG
TGCACAGAGTTAGCCCCAAGTATACGCCATTTGTCAATAGTAACTGATTCTGCATACCGCAAGGAGAAATATAGA
AACATATCTCGTAATGAGGTGTTTGAGAAAAGGTTGATGAAAGTTAAGTCAAGGAGTAAGTTGAGGTCACTGGTA
TTAATTGGGCAATATGATTCTCATTTTTTTAAATATTTCAAAGATGCTTTCAAGGAAGCACAACATCTGCGACTG
CTGCAGATCACTGCAACTTATGCTGATTCTGATTCATTTCTCTCCAGTTTGGTAAATTCTACACATCTCCGGTAT
CTGAAAATTGTGACCGAAGAATCCGGCAGAACTTTGCCCCGATCTCTAAGGAAGTATTACCATCTTCAAGTACTA
GATATTGGCTATAGATTTGGAATTCCCCGTATATCTAATGATATAAATAATCTTCTCAGCCTGCGGCATCTTGTT
GCATATGATGAAGTGTGTTCTTCCATTGCTAACATTGGTAAAATGACCTCACTTCAGGAACTAGGCAATTTTATT
GTTCAGAATAATTTAAGTGGTTTTGAGGTGACACAATTGAAATCCATGAACAAGCTTGTACAACTTAGTGTGTCT
CAGCTTGAAAATGTTAGAACTCAGGAGGAGGCATGTGGGGCAAAACTGAAAGACAAACAACACTTAGAAAAGCTA
CATTTGTCCTGGAAGGATGCATGGAATGGATATGACAGTGACGAAAGCTATGAAGATGAATACGGCAGTGATATG
AATATAGAAACAGAAGGGGAGGAACTGTCAGTTGGTGATGCCAATGGTGCCCAAAGCTTACAACATCACAGTAAT
ATAAGCTCTGAACTTGCTTCAAGTGAGGTGCTCGAAGGTCTTGAACCACATCACGGCCTCAAGTATCTACGGATA
TCTGGGTATAATGGATCTACCTCCCCAACTTGGCTTCCTTCTTCACTTACCTGTCTGCAAACACTTCATCTAGAA
AAATGTGGAAAATGGCAAATACTTCCTTTAGAAAGGCTAGGGTTACTTGTAAAGCTCGTGTTGATCAAAATGAGG
AATGCAACAGAACTCTCAATCCCTTCACTGGAGGAGCTTGTGTTAATTGCATTGCCAAGCTTGAACACATGCTCC
TGCACTTCCATCAGGAACTTGAACTCCAGTTTAAAGGTTCTGAAAATTAAGAATTGCCCTGTACTGAAGGTATTT
CCCTTGTTTGAGATTTCCCAGAAATTTGAAATCGAGCGGACGTCGTCATGGTTGCCCCATCTTAGCAAGCTTACC
ATCTATAATTGTCCTCTTTCCTGTGTGCACAGTTCTCTGCCACCTTCCGCAATCAGTGGTTATGGAGAATATGGA
AGGTGTACCCTTCCGCAATCACTTGAGGAACTTTACATCCATGAGTATTCTCAAGAAACTCTGCAGCCCTGCTTT
TCAGGGAACCTCACTCTCCTGAGAAAATTACATGTACTGGGAAACTCAAATTTAGTGTCTCTGCAGCTCCATTCC
TGCACAGCACTCGAAGAGTTGATAATTCAAAGCTGTGAGTCTCTTAGTTCTCTGGATGGCTTGCAATTGCTTGGC
AATCTCAGGTTGCTGCAGGCACATAGATGCCTCAGTGGTCATGGAGAAGATGGAAGGTGTATCCTTCCGCAATCA
CTTGAGGAACTTTACATCCATGAGTATTCTCAAGAAACTCTGCAGCCCTGCTTTTCAGGGAACCTCACTCTCCTG
AGAAAATTACATGTAATGGGAAACTCAAATTTAGTGTCTCTGCAGCTCCATTCCTGCACAGCACTCGAAGAGTTG
ATAATTCAAAGCTGTGAGTCTCTTAGTTCTCTGGATGGCTTGCAATTGCTTGGCAATCTCAGGTTGCTGCGGGCA
CATAGATGCCTCAGTGGTCATGGAGAAGATGGAAGGTGTATCCTTCCGCAATCACTTGAGGAACTTTACATCCAT
GAGTATTCTCAAGAAACTCTGCAGCCCTGCTTTTCAGGGAACCTCACTCTCCTGAGAAAATTACATGTACTGGAA
AACTCAAATTTAGTGTCTCTGCAGCTCCATTCCTGCACAGCACTCGAAGAGTTGATAATTCAAAGCTGTGAGTCT
CTTAGTTCTCTGGATGGCTTGCAATTGCTTGGCAATCTCAGGTTGCTGCAGGCACATAGATGCCTCAGTGGTCAT
GGAGAAGATGGAAGGTGTATCCTTCCGCAATCACTTGAGGAACTTTACATCCATGAGTATTCTCAAGAAACTCTG
CAGCCCTGCTTTTCAGGGAACCTCACTCTCCTGAGAAAATTACATGTACTGGGAAACTCAAATTTAGTGTCTCTG
CAGCTCCATTCCTGCACATCACTCGAAGAGTTGAAAATTCAAAGCTGTGAGTCTCTTAGTTCTCTGGATGGCTTG
CAATTGCTTGGCAATCTCAGGTTGCTGCAGGCACATAGATGCCTCAGTGGTCATGGAGAAGATGGAAGGTGTATC
CTTCCGCAATCACTTGAGGAACTTTTCATCAGTGAGTATTCTCTAGAAACTCTGCAGCCCTGCTTCCTGACGAAT
CTCACCTGCTTAAAACAATTAGAGGTATCAGGCACCACAAGTTTAAAATCTCTAGAACTGCAATCATGCACTGCA
CTCGAACATTTGAAGATTCAAGGTTGTGCGTCGCTTGCTACATTGGAGGGGTTGCAATTCCTCCACGCCCTCAGG
CATATGAAAGTATTCAGATGCCCTGGCTTGCCTCCATATTTGGGGAGTTCGTCAGAGCAGGGCTATGAGCTATGC
CCACGACTGGAAAGGCTCGACATCGATGACCCCTCTATCCTTACCACGTCGTTCTGCAAGCACCTCACCTCCCTC
CAACGCCTAGAGCTTAACTATTGCGGAAGTGAAGTGGCAAGACTAACGGATGAGCAAGAGAGAGCGCTTCAGCTC
CTCACGTCCCTGCAAGAGCTCCGGTTTAAGTATTGCTACAATCTCATAGATCTTCCTGCGGGGCTCCACAGCCTT
CCCTCCCTCAAGAGGTTGGAGATCCGGAGTTGCAGGAGCATCGCGAGGCTGCCGGAGAAGGGCCTCCCACCTTCG
TTCGAAGAACTGGATATCATCGCTTGCAGTAATGAGCTAGCTCAGCAGTGCAGAACTCTAGCAAGCACTCTGAAG
GTCAAAATTAATGGGGGATATGTGAACTGA
Xa2 protein SEQ ID NO: 4
MEEVEAGWLEGGIRWLAETILDNLDADKLDEWIRQIRLAADTEKLRAEIEKVDGVVAAVKGRAIGNRSLARSLGR
LRGLLYDADDAVDELDYFRLQQQVEGGVTTRFEAEETVGDGAEDEDDIPMDNTDVPEAVAAGSSKKRSKAWEHFT
TVEFTADGKDSKARCKYCHKDLCCTSKNGTSALRNHLNVCKRKRVTSTDQPVNPSSAGEGASNATGNSVGRKRMR
MDGTSTHHEAVSTHPWNKAELSNRIQCMTHQLEEAVNEVMRLCRSSSSNQSRQGTPPATNATTSSYLPEPIVYGR
AAEMETIKQLIMSNRSNGITVLPIVGNGGIGKTTLAQLVCKDLVIKSQFNVKIWVYVSDKFDVVKITRQILDHVS
NQSHEGISNLDTLQQDLEEQMKSKKFLIVLDDVWEIRTDDWKKLLAPLRPNDQVNSSQEEATGNMIILTTRIQSI
AKSLGTVQSIKLEALKDDDIWSLFKVHAFGNDKHDSSPGLQVLGKQIASELKGNPLAAKTVGSLLGTNLTIDHWD
SIIKSEEWKSLQQAYGIMQALKLSYDHLSNPLQQCVSYCSLFPKGYSFSKAQLIQIWIAQGFVEESSEKLEQKGW
KYLAELVNSGFLQQVESTRFSSEYFVMHDLMHDLAQKVSQTEYATIDGSECTELAPSIRHLSIVTDSAYRKEKYR
NISRNEVFEKRLMKVKSRSKLRSLVLIGQYDSHFFKYFKDAFKEAQHLRLLQITATYADSDSFLSSLVNSTHLRY
LKIVTEESGRTLPRSLRKYYHLQVLDIGYREGIPRISNDINNLLSLRHLVAYDEVCSSTANIGKMTSLQELGNFI
VQNNLSGFEVTQLKSMNKLVQLSVSQLENVRTQEEACGAKLKDKQHLEKLHLSWKDAWNGYDSDESYEDEYGSDM
NIETEGEELSVGDANGAQSLQHHSNISSELASSEVLEGLEPHHGLKYLRISGYNGSTSPTWLPSSLTCLQTLHLE
KCGKWQILPLERLGLLVKLVLIKMRNATELSIPSLEELVLIALPSLNTCSCTSIRNLNSSLKVLKIKNCPVLKVF
PLFEISQKFEIERTSSWLPHLSKLTIYNCPLSCVHSSLPPSAISGYGEYGRCTLPQSLEELYIHEYSQETLQPCF
SGNLTLLRKLHVLGNSNLVSLQLHSCTALEELIIQSCESLSSLDGLQLLGNLRLLQAHRCLSGHGEDGRCILPQS
LEELYIHEYSQETLQPCFSGNLTLLRKLHVMGNSNLVSLQLHSCTALEELIIQSCESLSSLDGLQLLGNLRLLRA
HRCLSGHGEDGRCILPQSLEELYIHEYSQETLQPCFSGNLTLLRKLHVLENSNLVSLQLHSCTALEELIIQSCES
LSSLDGLQLLGNLRLLQAHRCLSGHGEDGRCILPQSLEELYIHEYSQETLQPCFSGNLTLLRKLHVLGNSNLVSL
QLHSCTSLEELKIQSCESLSSLDGLQLLGNLRLLQAHRCLSGHGEDGRCILPQSLEELFISEYSLETLQPCFLTN
LTCLKQLEVSGTTSLKSLELQSCTALEHLKIQGCASLATLEGLQFLHALRHMKVERCPGLPPYLGSSSEQGYELC
PRLERLDIDDPSILTTSFCKHLTSLQRLELNYCGSEVARLTDEQERALQLLTSLQELRFKYCYNLIDLPAGLHSL
PSLKRLEIRSCRSIARLPEKGLPPSFEELDIIACSNELAQQCRTLASTLKVKINGGYVN
iTAL3a genomic sequence SEQ ID NO: 5
ATGGATCCCATTCGTTCGCGCACGCCAAGTCCTGCCCGCGAGCCTCTGCCCGGACCCCAACCGGATAGGGTTCAG
CCGACTGCAGATCGTGGGGTGTCTGCGCCTGCTGGCAGCCCTCTGGATGGCTTGCCCGCTCGGCGGACGGTGTCC
CGGACCCGGCTGCCATCTCCCCCTGCCCCCTTGCCTGCGTTCTCGGCGGGCAGCTCCACCGATCGGCTCCGTCCG
TTCGATCCGTCGCTTCCTGATACATCGCTTTTTGATTCGATGCCTGCCGTCGGCACGCCTCATACAGAGGCTGCC
CCAGCAGACACTTCGCCGGCCGCGCAGGTGGATCTACTCACGCTCGCGACAGTGGCGCAGCACCACGAGGCACTG
GTGGGCCATGGGTTTACACACGCGCACATCGTTGCGCTCAGCCAACACCCGGCAGCGTTAGGGACCGTTGCTGTC
ACGTATCAAGACATAATCACGGCGTTGCCAGAGGCGACACACGAAGACATCGTTGGCGTCGGCAAACAGTTGTCC
GGCGCACGCGCCCTGGAGGCCTTGCTCACGAAGGCGGGGGAGTTGAGAGGTCCGCCGTTACAGTTGGACACAGGC
CAACTTCTCAAGATTGCAAGACGTGGCGGCGTGACCGCAGTGGAGGCAGTGCATGCATGGCGCAATGCACTGACG
GGTGCCCCCCTGAACCTGACCCCGGACCAAGTGGTGGCCATCGCCAGCAATAGTGGCGGCAAGCAGGCGCTGGAG
ACGGTGCAGCGGCTGTTGCCGGTGCTGTGCCAGGACCATGGCCTGACCCCGGACCAGGTCGTGGCCATCGCCAGC
CACGATGGCGGCAAGCAGGCGCTGGAGACGGTGCAGCGGCTGTTGCCGGTGCTGTGCCAGGACCATGGCCTGACC
CCGGACCAGGTGGTGGCCATCGCCAGCAATGGCGGCGGCAAGCAGGCGCTGGAGACGGTACTGTGTCAGGCCCAT
GGCCTGACCCCGGCGCAGGTGGTGGCCATCGCCAGCAATGGCGGCGGCAAGCAGGCGCTGGAGACGGTGCAGCGG
CTGTTGCCGGTGCTGTGCCAGGCCCATGGCCTGACCCTGGACCAGGTCGTGGCCATTGCCAGCAATGGCGGCGGC
AAGCAGGCGCTGGAGACGGTGCAGCGGCTGTTGCCGGTGCTGTGCCAGGCCCATGGCCTGACCCCGGACCAGGTG
GTGGCCATCGCCAGCCACGATGGCGGCAAGCAGGCGCTGGAGACGGTGCAGCGGCTGTTGCCGGTGCTGTGCCAG
GCCCATGGCCTGACCCCGGCCCAGGTGGTGGCCATCGCCAGCCACGATGGCGGCAAGCAGGCGCTGGAGACGGTG
CAGCGGCTGTTGCCGGTGCTGTGCCAGGCCCATGGTCTGACCCTGGACCAGGTCGTGGCCATTGCCAGCAATGGC
GGCGGCAAGCAGGCGCTGGAGACGGTGCAGCGGCTGTTGCCGGTGCTGTGCCAGGCCCATGGTCTGACCCCGGAC
CAGGTCGTGGCCATCGCCAGCCACGATGGCGGCAAGCAGGCGCTGGAGACGGTGCAGCGGCTGTTGCCGGTGCTG
TGCCAGGACCATGGTCTGACCCCGGCGCAGGTGGTGGCCATCGCCAGCAATAACGGCGGCAAGCAGGCGCTGGAG
ACGGTGCAGCGGCTGTTGCCGGTGCTGTGCCAGGCCCATGGTCTGACCCTGGACCAGGTCGTGGCCATTGCCAGC
AATGGCGGCGGCAAGCAGGCGCTGGAGACGGTGCAGCGGCTGTTGCCGGTGCTGTGCCAGGCCCATGGTCTGACC
CCGGACCAGGTCGTGGCCATCGCCAGCCACGATGGCGGCAAGCAGGCGCTGGAGACGGTGCAGCGGCTGTTGCCG
GTGCTGTGCCAGGACCATGGTCTGACCCCGGCGCAGGTGGTGGCCATCGCCAGCAATAACGGCGGCAAGCAGGCG
CTGGAGACGGTGCAGCGGCTGTTGCCGGTGCTGTGCCAGGCCCATGGCCTGACCCCGGACCAGGTGGTGGCCATC
GCCAGCCACGATGGCGGCAAGCAGGCGCTGGAGACGGTGCAGCGGCTGTTGCCGGTGCTGTGCCAGGCCCATGGC
CTGACCCCGGACCAGGTCGTGGCCATTGCCAGCAATGGCGGCGGCAAGCAGGCGCTGGAGACGGTGCAGCGGCTG
TTGCCGGTGCTGTGCCAGGCCCATGGTCTGACCCCGGACCAGGTCGTGGCCATCGCCAGCCACGATGGCGGCAAG
CAGGCGCTGGAGACGGTGCAGCGGCTGTTGCCGGTGCTGTGCCAGGACCATGGCCTGACCCCGGACCAGGTCGTG
GCCATCGCCAGCAATATTGGCGGCAAGCAGGCGCTGGAGACGGTGCAGCGGCTGTTGCCGGTGCTGTGCCAGGAC
CATGGCCTGACCCCGGACCAGGTCGTGGCCATCGCCAGCAATGGCGGCAAGCAGGCGCTGGAGAGCATTGTTGCC
CAGTTATCTCGCCCTGATCCGGCGTTGGCCGCGTTGACCAACGACCACCTCGTCGCCTTGGCCTGCCTCGGCGGA
CGTCCTGCCCTGGATGCAGTGAAAAAGGGATTGCCGCACGCGCCGGAATTGATCAGAAGAGTCAATAGCCGTATT
GGCGAACGCACGTCCCATCGCGTTGCCGACCTCGCGCACGTGGTGCGCGTGCTTGGTTTTTTCCAGAGCCACTCC
CACCCAGCGCAAGCATTCGATGACGCCATGACGCAGTTCGGGATGAGCAGGCACGGGTTGGTACAGCTCTTTCGC
AGAGTGGGCGTCACCGAATTCGAAGCCCGCTGCGGAACTATCCCCCCAGCCTCGCAGCGTTGGGACCGTATCCTC
CAGGCATCAGGGACGAAAAGGGCCAAACCGTCCCCTACTTCAGCTCAGACGCCGGATCAGGCGTCTTTGCATGCA
TTCCCCGACTCGCTGGAGCGTGACCTTGATGCGCCCAGCCCAATGCACGAGGGAGATCAGACGCGGGCAAGCAGA
CGTAAACGGTCCTGA
iTal3a protein SEQ ID NO: 6
MDPIRSRTPSPAREPLPGPQPDRVQPTADRGVSAPAGSPLDGLPARRTVSRTRLPSPPAPLPAFSAGSSTDRLRP
FDPSLPDTSLFD SMPAVGTPHTEAAPADTSPAAQVDLLTLATVAQHHEALVGHGFTHAHIVALSQHPAALGTVAV
TYQDIITALPEATHEDIVGVGKQLSGARALEALLTKAGELRGPPLQLDTGQLLKIARRGGVTAVEAVHAWRNALT
GAPLNLTPDQVVAIASNSGGKQALETVQRLLPVLCQDHGLTPDQVVAIASHDGGKQALETVQRLLPVLCQDHGLT
PDQVVAIASNGGGKQALETVLCQAHGLTPAQVVAIASNGGGKQALETVQRLLPVLCQAHGLTLDQVVAIASNGGG
KQALETVQRLLPVLCQAHGLTPDQVVAIASHDGGKQALETVQRLLPVLCQAHGLTPAQVVAIASHDGGKQALETV
QRLLPVLCQAHGLTLDQVVAIASNGGGKQALETVQRLLPVLCQAHGLTPDQVVAIASHDGGKQALETVQRLLPVL
CQDHGLTPAQVVAIASNNGGKQALETVQRLLPVLCQAHGLTLDQVVAIASNGGGKQALETVQRLLPVLCQAHGLT
PDQVVAIASHDGGKQALETVQRLLPVLCQDHGLTPAQVVAIASNNGGKQALETVQRLLPVLCQAHGLTPDQVVAI
ASHDGGKQALETVQRLLPVLCQAHGLTPDQVVAIASNGGGKQALETVQRLLPVLCQAHGLTPDQVVAIASHDGGK
QALETVQRLLPVLCQDHGLTPDQVVAIASNIGGKQALETVQRLLPVLCQDHGLTPDQVVAIASNGGKQALESIVA
QLSRPDPALAALTNDHLVALACLGGRPALDAVKKGLPHAPELIRRVNSRIGERTSHRVADLAHVVRVLGFFQSHS
HPAQAFDDAMTQFGMSRHGLVQLFRRVGVTEFEARCGTIPPASQRWDRILQASGTKRAKPSPTSAQTPDQASLHA
FPDSLERDLDAPSPMHEGDQTRASRRKRS*
iTal3b genomic sequence SEQ ID NO: 7
ATGGATCCCATTCGTTCGCGCACGCCAAGTCCTGCCCGCGAGCCTCTGCCCGGACCCCAACCGGATAGGGTTCAG
CCGACTGCAGATCGTGGGGTGTCTGCGCCTGCTGGCAGCCCTCTGGATGGCTTGCCCGCTCGGCGGACGGTGTCC
CGGACCCGGCTGCCATCTCCCCCTGCCCCCTTGCCTGCGTTCTCGGCGGGCAGCTCCACCGATCGGCTCCGTCCG
TTCGATCCGTCGCTTCCTGATACATCGCTTTTTGATTCGATGCCTGCCGTCGGCACGCCTCATACAGAGGCTGCC
CCAGCAGACACTTCGCCGGCCGCGCAGGTGGATCTACTCACGCTCGCGACAGTGGCGCAGCACCACGAGGCACTG
GTGGGCCATGGGTTTACACACGCGCACATCGTTGCGCTCAGCCAACACCCGGCAGCGTTAGGGACCGTTGCTGTC
ACGTATCAAGACATAATCACGGCGTTGCCAGAGGCGACACACGAAGACATCGTTGGCGTCGGCAAACAGTTGTCC
GGCGCACGCGCCCTGGAGGCCTTGCTCACGAAGGCGGGGGAGTTGAGAGGTCCGCCGTTACAGTTGGACACAGGC
CAACTTCTCAAGATTGCAAGACGTGGCGGCGTGACCGCAGTGGAGGCAGTGCATGCATGGCGCAATGCACTGACG
GGTGCCCCCCTGAACCTGACCCCGGACCAAGTGGTGGCCATCGCCAGCAATAGTGGCGGCAAGCAGGCGCTGGAG
ACGGTGCAGCGGCTGTTGCCGGTGCTGTGCCAGGACCATGGCCTGACCCCGGACCAGGTCGTGGCCATCGCCAGC
CACGATGGCGGCAAGCAGGCGCTGGAGACGGTGCAGCGGCTGTTGCCGGTGCTGTGCCAGGACCATGGCCTGACC
CCGGACCAGGTGGTGGCCATCGCCAGCAATGGCGGCGGCAAGCAGGCGCTGGAGACGGTGCAGCGGCTGTTGCCG
GTGCTGTGCCAGGACCATGGCCTGACCCCGGACCAGGTGGTGGCCATCGCCAGCAATGGCGGCGGCAAGCAGGCG
CTGGAGACGGTACTGTGCCAGGCCCATGGCCTGACCCCGGCGCAGGTGGTGGCCATCGCCAGCAATGGCGGCGGC
AAGCAGGCGCTGGAGACGGTGCAGCGGCTGTTGCCGGTGCTGTGCCAGGCCCATGGCCTGACCCTGGACCAGGTC
GTGGCCATTGCCAGCAATGGCGGCGGCAAGCAGGCGCTGGAGACGGTGCAGCGGCTGTTGCCGGTGCTGTGCCAG
GCCCATGGCCTGACCCCGGCGCAGGTGGTGGCCATCGCCAGCAATGGCGGCGGCAAGCAGGCGCTGGAGACGGTG
CAGCGGCTGTTGCCGGTGCTGTGCCAGGCCCATGGCCTGACCCCGGCCCAGGTGGTGGCCATCGCCAGCCACGAT
GGCGGCAAGCAGGCGCTGGAGACGGTGCAGCGGCTGTTGCCGGTGCTGTGCCAGGCCCATGGTCTGACCCTGGAC
CAGGTAGTGGCCATTGCCAGCCACGATGGCGGCAAGCAGGCGCTGGAGACGGTGCAGCGGCTGTTGCCGGTGCTG
TGCCAGGCCCATGGTCTGACCCTGGACCAGGTCGTGGCCATCGCCAGCCACGATGGCGGCAAGCAGGCGCTGGAG
ACGGTGCAGCGGCTGTTGCCGGTGCTGTGCCAGGACCATGGTCTGACCCCGGCGCAGGTGGTGGCCATCGCCAGC
AATAACGGCGGCAAGCAGGCGCTGGAGACGGTGCAGCGGCTGTTGCCGGTGCTGTGCCAGGACCATGGCCTGACC
CCGGACCAGGTGGTGGCCATCGCCAGCCACGATGGCGGCAAGCAGGCGCTGGAGACGATGCAGCGGCTGTTGCCG
GTGCTGTGCCAGGCCCATGGCCTGACCCCGGACCAGGTCGTGGCCATCGCCAGCAATGGCGGCGGCAAGCAGGCG
CTGGAGACGGTGCAGCGGCTGTTGCCGGTGCTGTGCCAGGCCCATGGCCTGACCCCGGACCAGGTCGTGGCCATC
GCCAGCCACGATGGCGGCAAGCAGGCGCTGGAGACGGTGCAGCGGCTGTTGCCGGTGCTGTGCCAGGACCATGGC
CTGACCCCGGACCAGGTCGTGGCCATCGCCAGCCACGATGGCGGCAAGCAGGCGCTGGAGACGGTGCAACGGCTG
TTGCAGGTGCTGTGCCAGGACCATGGCCTGACCCCGGACCAGGTCGTGGCCATCGCCAGCCACGATGGCGGCAAG
CAGGCGCTGGAGACGGTGCAACGGCTGTTGCCGGTGCTGTGCCAGGACCATGGCCTGACCCCGGACCAGGTCGTG
GCCATCGCCAGCCACGATGGCGGCAAGCAGGCGCTGGAGACGGTGCAGCGGCTGTTGCCGGTGCTGTGCCAGGAC
CATGGCCTGACCCCGGACCAGGTCGTGGCCATCGCCAGCAATGGCGGCAAGCAGGCGCTGGAGAGCATTGTTGCC
CAGTTATCTCGCCGTGATCCGGCGTTGGCCGCGTTGACCAACGACCAACTCGTCGCCTTGGCCTGCCTCGGCGGA
CGTCCTGCCCCGCATTCAAGGAAGAGGAAATCGCATGATTGA
iTal3b protein SEQ ID NO: 8
MDPIRSRTPSPAREPLPGPQPDRVQPTADRGVSAPAGSPLDGLPARRTVSRTRLPSPPAPLPAFSAGSSTDRLRP
FDPSLPDTSLFDSMPAVGTPHTEAAPADTSPAAQVDLLTLATVAQHHEALVGHGFTHAHIVALSQHPAALGTVAV
TYQDIITALPEATHEDIVGVGKQLSGARALEALLTKAGELRGPPLQLDTGQLLKIARRGGVTAVEAVHAWRNALT
GAPLNLTPDQVVAIASNSGGKQALETVQRLLPVLCQDHGLTPDQVVAIASHDGGKQALETVQRLLPVLCQDHGLT
PDQVVAIASNGGGKQALETVQRLLPVLCQDHGLTPDQVVAIASNGGGKQALETVLCQAHGLTPAQVVAIASNGGG
KQALETVQRLLPVLCQAHGLTLDQVVAIASNGGGKQALETVQRLLPVLCQAHGLTPAQVVAIASNGGGKQALETV
QRLLPVLCQAHGLTPAQVVAIASHDGGKQALETVQRLLPVLCQAHGLTLDQVVAIASHDGGKQALETVQRLLPVL
CQAHGLTLDQVVAIASHDGGKQALETVQRLLPVLCQDHGLTPAQVVAIASNNGGKQALETVQRLLPVLCQDHGLT
PDQVVAIASHDGGKQALETMQRLLPVLCQAHGLTPDQVVAIASNGGGKQALETVQRLLPVLCQAHGLTPDQVVAI
ASHDGGKQALETVQRLLPVLCQDHGLTPDQVVAIASHDGGKQALETVQRLLQVLCQDHGLTPDQVVAIASHDGGK
QALETVQRLLPVLCQDHGLTPDQVVAIASHDGGKQALETVQRLLPVLCQDHGLTPDQVVAIASNGGKQALESIVA
QLSRRDPALAALTNDQLVALACLGGRPAPHSRKRKSHD

What is claimed is:

1. A genetically modified plant with improved Xanthomonas tolerance, compared to the Xanthomonas tolerance of a corresponding plant with no such modification; said modified plant having modulated Xa1, Xa2, iTAL3a and/or iTAL3b activity.

2. The genetically modified plant of claim 1 wherein said modulated activity includes an increase in activity/expression of a Xa1, or Xa2 gene or protein encoded thereby.

3. The genetically modified plant of claim 1 wherein said modulated activity includes a decrease in activity/expression of iTAL3a and/or iTAL3b activity.

4. The genetically modified plant of claim 1, said plant comprising a heterologous nucleotide sequence for modulating Xanthomonas resistance comprising a member selected from:
(a) a polynucleotide, having at least about 95%, at least about 99%, about 99.5% or more sequence identity to SEQ ID NOS:1, 3, 5, or 7,
(b) a polynucleotide, or a complement thereof, encoding a polypeptide sequence of SEQ ID NO: 2, 4, 6, or 8, or a subsequence thereof, or a conservative variation thereof;
(c) a polynucleotide, or a complement thereof, that hybridizes under stringent conditions over substantially the entire length of a polynucleotide subsequence comprising at least 100 contiguous nucleotides of SEQ ID NO: 1, 3, 5 or 7, or that hybridizes to a polynucleotide sequence of (a) or (b); and, (d) a polynucleotide that is at least about 85% identical to a polynucleotide sequence of (a), (b) or (c) wherein polynucleotide includes at least one base change so as not to be the genomic sequence.

5. The plant of claim 4 wherein said resistance nucleic acid is operably linked to a heterologous promoter.

6. A modified plant with improved plant pathogen resistance particularly Xanthomonas tolerance compared to the Xanthomonas tolerance of a corresponding plant with no such modification; said modified plant comprising an antagonist, wherein in said antagonist reduces the expression/activity of Tal3a and/or Tal3b.

7. An isolated nucleic acid molecule, said molecule encoding a Xanthomonas resistance protein wherein said nucleic acid molecule comprises a nucleotide sequence selected from the group consisting of:
(a) a polynucleotide, having at least about 95%, at least about 99%, about 99.5% or more sequence identity to SEQ ID NOS:1, 3, 5, or 7,
(b) a polynucleotide, or a complement thereof, encoding a polypeptide sequence of SEQ ID NO: 2, 4, 6, or 8, or a subsequence thereof, or a conservative variation thereof;
(c) a polynucleotide, or a complement thereof, that hybridizes under stringent conditions over substantially the entire length of a polynucleotide subsequence comprising at least 100 contiguous nucleotides of SEQ ID NO: 1, 3, 5 or 7, or that hybridizes to a polynucleotide sequence of (a) or (b); and, (d) a polynucleotide that is at least about 85% identical to a polynucleotide sequence of (a), (b) or (c) wherein polynucleotide includes at least one base change so as not to be the genomic sequence and further wherein said nucleotide sequence encodes a protein for Xanthomonas resistance.

8. A vector comprising the nucleic acid molecule of claim 7.

9. A vector comprising the nucleic acid sequence of claim 7 operably linked to a heterologous promoter.

10. A plant cell having stably incorporated in its genome the vector of claim 9.

11. A plant cell having stably incorporated in its genome the nucleic acid molecule of claim 7.

12. The plant cell of claim 10, wherein said plant cell is selected from the group consisting of rice, pepper, tomato, beans, cotton, cucumber, cabbage, barley, oats, wheat, corn and citrus.

13. A method for conferring or improving Xanthomaonas resistance in a plant, said method comprising:
transforming said plant with a nucleic acid molecule comprising a heterologous sequence operably linked to a heterologous promoter that induces transcription of said heterologous sequence in a plant cell; and
regenerating stably transformed plants, wherein said heterologous sequence comprises a nucleic acid molecule that encodes one or more resistance protein sequences of Xa1, Xa2, iTAL3a and/or iTAL3b activity.

14. The method of claim 13 wherein said nucleic acid encodes one or more of: SEQ ID NO: 1, 3, 5 or 7.

15. The method of claim 13 wherein said nucleic acid sequence includes:
selected from the group consisting of:
(a) a polynucleotide, having at least about 95%, at least about 99%, about 99.5% or more sequence identity to SEQ ID NOS:1, 3, 5, or 7,
(b) a polynucleotide, or a complement thereof, encoding a polypeptide sequence of SEQ ID NO: 2, 4, 6, or 8, or a subsequence thereof, or a conservative variation thereof;
(c) a polynucleotide, or a complement thereof, that hybridizes under stringent conditions over substantially the entire length of a polynucleotide subsequence comprising at least 100 contiguous nucleotides of SEQ ID NO: 1, 3, 5 or 7, or that hybridizes to a polynucleotide sequence of (a) or (b); and, (d) a polynucleotide that is at least about 85% identical to a polynucleotide sequence of (a), (b) or (c).

16. The method of 13, wherein said plant is selected from the group consisting of rice, pepper, tomato, beans, cotton, cucumber, cabbage, barley, oats, wheat, corn and citrus.

17. An isolated polypeptide having resistance to Xanthomonas selected from the group consisting of:
(a) a polypeptide comprising at least 90% or 95% sequence identity to SEQ ID NO: 2, 4, 6, or 8 or fragment thereof (b) a polypeptide encoded by a nucleic acid of the present invention or fragment thereof, and (c) a polypeptide comprising a Xanthomonas resistance activity and comprising conserved structural domain motifs of the same.

18. A nucleotide construct comprising:
a nucleic acid molecule of claim 7, wherein said nucleic acid molecule is operably linked to a heterologous promoter that drives expression in a plant cell.

19. A method for conferring or improving Xanthomonas resistance of a plant, said method comprising:
stably introducing into the genome of a plant, at least one nucleotide construct comprising a resistance nucleic acid molecule operably linked to a heterologous promoter that drives expression in a plant cell, wherein said nucleic acid molecule encodes a polypeptide selected from the group consisting of: Xa1, Xa2, iTAL3a and/or iTAL3b activity.

20. The method of claim 19 wherein said nucleic acid molecule serves to decrease iTAL3a and/or iTAL3b activity.

21. The method of claim 19 wherein said construct serves to increase Xa1, and/or Xa2 activity.

22. A method of plant breeding for Xanthomonas resistance comprising:
identifying a plant with a resistance nucleic acid encoding an exogenous Xa1, Xa2, iTAL3a and/or iTAL3b protein, conservatively modified variants thereof, a Xa1, Xa2, iTAL3a and/or iTAL3b protein with one or more amino acid changes; or the protein product thereof;
selecting said resistant plant for use a parent plant;
crossing said parent plant with itself or a second plant, so that the Xanthomonas resistance trait is passed to progeny seed; and
harvesting progeny seed from said parent plant.

23. A plant or plant part produced by the method of claim 22.


SEQUENCE LISTING (95 sequences)

SEQ ID NO: 1 - DNA, 5409 bp, Oryza sativa
atggaggaggtggaagccggttggctggagggcgggatcaggtggctggcggagaccatcctggataacctggacgccgacaagctggatgaatggattcgccagattaggctcgccgctgacaccgagaagctacgggctgagatcgagaaggtggatggggtggtggctgccgtgaaggggagggcgatcgggaacaggtcgctggcccgatcgctcggccgtctcagggggttgctgtacgacgccgacgatgcggtcgacgagctcgactacttcaggctccagcagcaggtcgagggaggagttactacacggtttgaggctgaagagacggtcggagatggagcagaggacgaggacgatattccgatggacaatactgatgtaccggaggcagtggcggcaggcagcagcaagaaacggtccaaggcatgggaacactttactaccgtagagttcactgctgacgggaaggattctaaagcacggtgcaagtactgccacaaggacctatgttgcacatctaagaacgggacatcagctttgcgcaaccatctcaatgtttgcaagaggaaacgtgtaacaagtactgaccaaccggtaaatccatcaagtgccggtgagggtgcatcaaatgcaactggtaattcagttggcagaaaaaggatgagaatggatgggacttcaacacaccacgaggcagttagcacgcacccttggaacaaggctgaactttccaacaggatccaatgcatgactcatcagttagaagaggctgtaaatgaggttatgaggctatgtcgatcctcaagttcaaaccagagtcgacagggtacaccaccggccacaaatgcaacaacatcgtcttatcttccggagcccatagtgtatgggagggctgcagagatggaaaccatcaaacagctgatcatgagcaatagatctaatggcataaccgtcctgccaattgtaggcaatggagggataggaaaaaccactttggcgcaactggtctgcaaagatctggtaattaaaagtcagtttaatgttaagatatgggtgtatgtatctgataaatttgatgtagttaagattacaaggcagattttggatcatgtctccaaccagagccacgaaggaataagcaaccttgatacgcttcagcaggatcttgaggaacaaatgaaatctaagaagttcctcattgtcttagatgatgtgtgggaaatccgtacagatgactggaaaaaactactggctcctttaagacctaatgatcaggtgaattcatcacaggaagaggcaacaggtaatatgataattttgacaactcgtatacagagtattgccaaaagtcttggaacagtacaatcaattaagttagaagctctgaaagatgacgatatatggtcactatttaaagtgcatgcttttggtaatgataaacatgatagtagtccaggcttacaggttcttgggaagcaaattgctagcgagctaaaaggcaacccactggcagcaaaaactgtgggttcactattaggaacgaatcttaccatcgatcattgggatagcattataaagagtgaagaatggaaatccctgcaacaagcttatggcatcatgcaagcgctgaagttgagctatgatcatctatccaaccccttacagcaatgcgtctcttattgttctcttttccccaagggttattctttcagcaaagcacaactaatacaaatatggattgctcaaggatttgtggaagaatccagtgagaagttggagcagaaaggatggaaatatctagctgagttggtaaattcgggtttccttcagcaagttgaaagcacacggttttcatcagaatattttgttatgcacgatcttatgcatgatttagcgcaaaaggtttcacaaacagaatatgcaactatagatggctcagagtgcacagagttagccccaagtatacgccatttgtcaatagtaactgattctgcataccgcaaggagaaatatagaaacatatctcgtaatgaggtgtttgagaaaaggttgatgaaagttaagtcaaggagtaagttgaggtcactggtattaattgggcaatatgattctcatttttttaaatatttcaaagatgctttcaaggaagcacaacatctgcgactgctgcagatcactgcaacttatgctgattctgattcatttctctccagtttggtaaattctacacatctccggtatctgaaaattgtgaccgaagaatccggcagaactttgccccgatctctaaggaagtattaccatcttcaagtactagatattggctatagatttggaattccccgtatatctaatgatataaataatcttctcagcctgcggcatcttgttgcatatgatgaagtgtgttcttccattgctaacattggtaaaatgacctcacttcaggaactaggcaattttattgttcagaataatttaagtggttttgaggtgacacaattgaaatccatgaacaagcttgtacaacttagtgtgtctcagcttgaaaatgttagaactcaggaggaggcatgtggggcaaaactgaaagacaaacaacacttagaaaagctacatttgtcctggaaggatgcatggaatggatatgacagtgacgaaagctatgaagatgaatacggcagtgatatgaatatagaaacagaaggggaggaactgtcagttggtgatgccaatggtgcccaaagcttacaacatcacagtaatataagctctgaacttgcttcaagtgaggtgctcgaaggtcttgaaccacatcacggcctcaagtatctacggatatctgggtataatggatctacctccccaacttggcttccttcttcacttacctgtctgcaaacacttcatctagaaaaatgtggaaaatggcaaatacttcctttagaaaggctagggttacttgtaaagctcgtgttgatcaaaatgaggaatgcaacagaactctcaatcccttcactggaggagcttgtgttaattgcattgccaagcttgaacacatgctcctgcacttccatcaggaacttgaactccagtttaaaggttctgaaaattaagaattgccctgtactgaaggtatttcccttgtttgagatttcccagaaatttgaaatcgagcggacgtcgtcatggttgccccatcttagcaagcttaccatctataattgtcctctttcctgtgtgcacagttctctgccaccttccgcaatcagtggttatggagaatatggaaggtgtacccttccgcaatcacttgaggaactttacatccatgagtattctcaagaaactctgcagccctgcttttcagggaacctcactctcctgagaaaattacatgtactgggaaactcaaatttagtgtctctgcagctccattcctgcacagcactcgaagagttgataattcaaagctgtgagtctcttagttctctggatggcttgcaattgcttggcaatctcaggttgctgcgggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtactgggaaactcaaatttagtgtctctgcagctccattcctgcacagcactcgaagagttgataattcaaagctgtgagtctcttagttctctggatggcttgcaattgcttggcaatctcaggttgctgcgggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtactgggaaactcaaatttcgtgtctctgcagctccattcctgcacagcactcgaagagttgataattcaaagctgtgagtctcttagttctctggatggcttgcaattgcttggcaatctcaggttgctgcaggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtacttggaaactcaaatttagtgtctctgcagctccattcctgcacagcactcgaagagttgataattcaaagctgtgagtctcttagttctctggatggcttgcaattgcttggcaatctcaggttgctgcaggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtactgggaaactcaaatttagtgtctctgcagctccattcctgcacatcactcgaagagttgaaaattcaaagctgtgagtctcttagttctctggatggcttgcaattgcttggcaatctcaggttgctgcaggcacatagatgcctcagtggtcatggagaagatggaaggtgtatccttccgcaatcacttgaggaacttttcatcagtgagtattctctagaaactctgcagccctgcttcctgacgaatctcacctgcttaaaacaattagaggtatcaggcaccacaagtttaaaatctctagaactgcaatcatgcactgcactcgaacatttgaagattcaaggttgtgcgtcgcttgctacattggaggggttgcaattcctccacgccctcaggcatatgaaagtattcagatgccctggcttgcctccatatttggggagttcgtcagagcagggctatgagctatgcccacgactggaaaggctcgacatcgatgacccctctatccttaccacgtcgttctgcaagcacctcacctccctccaacgcctagagcttaactattgcggaagtgaagtggcaagactaacggatgagcaagagagagcgcttcagctcctcacgtccctgcaagagctccggtttaagtattgctacaatctcatagatcttcctgcggggctccacagccttccctccctcaagaggttggagatccggagttgcaggagcatcgcgaggctgccggagaagggcctcccaccttcgttcgaagaactggatatcatcgcttgcagtaatgagctagctcagcagtgcagaactctagcaagcactctgaaggtcaaaattaatgggggatatgtgaactga

SEQ ID NO: 2 - protein, 1802 aa, Oryza sativa
MEEVEAGWLEGGIRWLAETILDNLDADKLDEWIRQIRLAADTEKLRAEIEKVDGVVAAVKGRAIGNRSLARSLGRLRGLLYDADDAVDELDYFRLQQQVEGGVTTRFEAEETVGDGAEDEDDIPMDNTDVPEAVAAGSSKKRSKAWEHFTTVEFTADGKDSKARCKYCHKDLCCTSKNGTSALRNHLNVCKRKRVTSTDQPVNPSSAGEGASNATGNSVGRKRMRMDGTSTHHEAVSTHPWNKAELSNRIQCMTHQLEEAVNEVMRLCRSSSSNQSRQGTPPATNATTSSYLPEPIVYGRAAEMETIKQLIMSNRSNGITVLPIVGNGGIGKTTLAQLVCKDLVIKSQFNVKIWVYVSDKFDVVKITRQILDHVSNQSHEGISNLDTLQQDLEEQMKSKKFLIVLDDVWEIRTDDWKKLLAPLRPNDQVNSSQEEATGNMIILTTRIQSIAKSLGTVQSIKLEALKDDDIWSLFKVHAFGNDKHDSSPGLQVLGKQIASELKGNPLAAKTVGSLLGTNLTIDHWDSIIKSEEWKSLQQAYGIMQALKLSYDHLSNPLQQCVSYCSLFPKGYSFSKAQLIQIWIAQGFVEESSEKLEQKGWKYLAELVNSGFLQQVESTRFSSEYFVMHDLMHDLAQKVSQTEYATIDGSECTELAPSIRHLSIVTDSAYRKEKYRNISRNEVFEKRLMKVKSRSKLRSLVLIGQYDSHFFKYFKDAFKEAQHLRLLQITATYADSDSFLSSLVNSTHLRYLKIVTEESGRTLPRSLRKYYHLQVLDIGYRFGIPRISNDINNLLSLRHLVAYDEVCSSIANIGKMTSLQELGNFIVQNNLSGFEVTQLKSMNKLVQLSVSQLENVRTQEEACGAKLKDKQHLEKLHLSWKDAWNGYDSDESYEDEYGSDMNIETEGEELSVGDANGAQSLQHHSNISSELASSEVLEGLEPHHGLKYLRISGYNGSTSPTWLPSSLTCLQTLHLEKCGKWQILPLERLGLLVKLVLIKMRNATELSIPSLEELVLIALPSLNTCSCTSIRNLNSSLKVLKIKNCPVLKVFPLFEISQKFEIERTSSWLPHLSKLTIYNCPLSCVHSSLPPSAISGYGEYGRCTLPQSLEELYIHEYSQETLQPCFSGNLTLLRKLHVLGNSNLVSLQLHSCTALEELIIQSCESLSSLDGLQLLGNLRLLRAHRCLSGHGEDGRCILPQSLEELYIHEYSQETLQPCFSGNLTLLRKLHVLGNSNLVSLQLHSCTALEELIIQSCESLSSLDGLQLLGNLRLLRAHRCLSGHGEDGRCILPQSLEELYIHEYSQETLQPCFSGNLTLLRKLHVLGNSNFVSLQLHSCTALEELIIQSCESLSSLDGLQLLGNLRLLQAHRCLSGHGEDGRCILPQSLEELYIHEYSQETLQPCFSGNLTLLRKLHVLGNSNLVSLQLHSCTALEELIIQSCESLSSLDGLQLLGNLRLLQAHRCLSGHGEDGRCILPQSLEELYIHEYSQETLQPCFSGNLTLLRKLHVLGNSNLVSLQLHSCTSLEELKIQSCESLSSLDGLQLLGNLRLLQAHRCLSGHGEDGRCILPQSLEELFISEYSLETLQPCFLTNLTCLKQLEVSGTTSLKSLELQSCTALEHLKIQGCASLATLEGLQFLHALRHMKVFRCPGLPPYLGSSSEQGYELCPRLERLDIDDPSILTTSFCKHLTSLQRLELNYCGSEVARLTDEQERALQLLTSLQELRFKYCYNLIDLPAGLHSLPSLKRLEIRSCRSIARLPEKGLPPSFEELDIIACSNELAQQCRTLASTLKVKINGGYVN

SEQ ID NO: 3 - DNA, 5130 bp, Oryza sativa
atggaggaggtggaagccggttggctggagggcgggatcaggtggctggcggagaccatcctggataacctggacgccgacaagctggatgaatggattcgccagattaggctcgccgctgacaccgagaagctacgggctgagatcgagaaggtggatggggtggtggctgccgtgaaggggagggcgatcgggaacaggtcgctggcccgatcgctcggccgtctcagggggttgctgtacgacgccgacgatgcggtcgacgagctcgactacttcaggctccagcagcaggtcgagggaggagttactacacggtttgaggctgaagagacggtcggagatggagcagaggacgaggacgatattccgatggacaatactgatgtaccggaggcagtggcggcaggcagcagcaagaaacggtccaaggcatgggaacactttactaccgtagagttcactgctgacgggaaggattctaaagcacggtgcaagtactgccacaaggacctatgttgcacatctaagaacgggacatcagctttgcgcaaccatctcaatgtttgcaagaggaaacgtgtaacaagtactgaccaaccggtaaatccatcaagtgccggtgagggtgcatcaaatgcaactggtaattcagttggcagaaaaaggatgagaatggatgggacttcaacacaccacgaggcagttagcacgcacccttggaacaaggctgaactttccaacaggatccaatgcatgactcatcagttagaagaggctgtaaatgaggttatgaggctatgtcgatcctcaagttcaaaccagagtcgacagggtacaccaccggccacaaatgcaacaacatcgtcttatcttccggagcccatagtgtatgggagggctgcagagatggaaaccatcaaacagctgatcatgagcaatagatctaatggcataaccgtcctgccaattgtaggcaatggagggataggaaaaaccactttggcgcaactggtctgcaaagatctggtaattaaaagtcagtttaatgttaagatatgggtgtatgtatctgataaatttgatgtagttaagattacaaggcagattttggatcatgtctccaaccagagccacgaaggaataagcaaccttgatacgcttcagcaggatcttgaggaacaaatgaaatctaagaagttcctcattgtcttagatgatgtgtgggaaatccgtacagatgactggaaaaaactactggctcctttaagacctaatgatcaggtgaattcatcacaggaagaggcaacaggtaatatgataattttgacaactcgtatacagagtattgccaaaagtcttggaacagtacaatcaattaagttagaagctctgaaagatgacgatatatggtcactatttaaagtgcatgcttttggtaatgataaacatgatagtagtccaggcttacaggttcttgggaagcaaattgctagcgagctaaaaggcaacccactggcagcaaaaactgtgggttcactattaggaacgaatcttaccatcgatcattgggatagcattataaagagtgaagaatggaaatccctgcaacaagcttatggcatcatgcaagcgctgaagttgagctatgatcatctatccaaccccttacagcaatgcgtctcttattgttctcttttccccaagggttattctttcagcaaagcacaactaatacaaatatggattgctcaaggatttgtggaagaatccagtgagaagttggagcagaaaggatggaaatatctagctgagttggtaaattcgggtttccttcagcaagttgaaagcacacggttttcatcagaatattttgttatgcacgatcttatgcatgatttagcgcaaaaggtttcacaaacagaatatgcaactatagatggctcagagtgcacagagttagccccaagtatacgccatttgtcaatagtaactgattctgcataccgcaaggagaaatatagaaacatatctcgtaatgaggtgtttgagaaaaggttgatgaaagttaagtcaaggagtaagttgaggtcactggtattaattgggcaatatgattctcatttttttaaatatttcaaagatgctttcaaggaagcacaacatctgcgactgctgcagatcactgcaacttatgctgattctgattcatttctctccagtttggtaaattctacacatctccggtatctgaaaattgtgaccgaagaatccggcagaactttgccccgatctctaaggaagtattaccatcttcaagtactagatattggctatagatttggaattccccgtatatctaatgatataaataatcttctcagcctgcggcatcttgttgcatatgatgaagtgtgttcttccattgctaacattggtaaaatgacctcacttcaggaactaggcaattttattgttcagaataatttaagtggttttgaggtgacacaattgaaatccatgaacaagcttgtacaacttagtgtgtctcagcttgaaaatgttagaactcaggaggaggcatgtggggcaaaactgaaagacaaacaacacttagaaaagctacatttgtcctggaaggatgcatggaatggatatgacagtgacgaaagctatgaagatgaatacggcagtgatatgaatatagaaacagaaggggaggaactgtcagttggtgatgccaatggtgcccaaagcttacaacatcacagtaatataagctctgaacttgcttcaagtgaggtgctcgaaggtcttgaaccacatcacggcctcaagtatctacggatatctgggtataatggatctacctccccaacttggcttccttcttcacttacctgtctgcaaacacttcatctagaaaaatgtggaaaatggcaaatacttcctttagaaaggctagggttacttgtaaagctcgtgttgatcaaaatgaggaatgcaacagaactctcaatcccttcactggaggagcttgtgttaattgcattgccaagcttgaacacatgctcctgcacttccatcaggaacttgaactccagtttaaaggttctgaaaattaagaattgccctgtactgaaggtatttcccttgtttgagatttcccagaaatttgaaatcgagcggacgtcgtcatggttgccccatcttagcaagcttaccatctataattgtcctctttcctgtgtgcacagttctctgccaccttccgcaatcagtggttatggagaatatggaaggtgtacccttccgcaatcacttgaggaactttacatccatgagtattctcaagaaactctgcagccctgcttttcagggaacctcactctcctgagaaaattacatgtactgggaaactcaaatttagtgtctctgcagctccattcctgcacagcactcgaagagttgataattcaaagctgtgagtctcttagttctctggatggcttgcaattgcttggcaatctcaggttgctgcaggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtaatgggaaactcaaatttagtgtctctgcagctccattcctgcacagcactcgaagagttgataattcaaagctgtgagtctcttagttctctggatggcttgcaattgcttggcaatctcaggttgctgcgggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtactggaaaactcaaatttagtgtctctgcagctccattcctgcacagcactcgaagagttgataattcaaagctgtgagtctcttagttctctggatggcttgcaattgcttggcaatctcaggttgctgcaggcacatagatgcctcagtggtcatggagaagatggaaggtgtatccttccgcaatcacttgaggaactttacatccatgagtattctcaagaaactctgcagccctgcttttcagggaacctcactctcctgagaaaattacatgtactgggaaactcaaatttagtgtctctgcagctccattcctgcacatcactcgaagagttgaaaattcaaagctgtgagtctcttagttctctggatggcttgcaattgcttggcaatctcaggttgctgcaggcacatagatgcctcagtggtcatggagaagatggaaggtgtatccttccgcaatcacttgaggaacttttcatcagtgagtattctctagaaactctgcagccctgcttcctgacgaatctcacctgcttaaaacaattagaggtatcaggcaccacaagtttaaaatctctagaactgcaatcatgcactgcactcgaacatttgaagattcaaggttgtgcgtcgcttgctacattggaggggttgcaattcctccacgccctcaggcatatgaaagtattcagatgccctggcttgcctccatatttggggagttcgtcagagcagggctatgagctatgcccacgactggaaaggctcgacatcgatgacccctctatccttaccacgtcgttctgcaagcacctcacctccctccaacgcctagagcttaactattgcggaagtgaagtggcaagactaacggatgagcaagagagagcgcttcagctcctcacgtccctgcaagagctccggtttaagtattgctacaatctcatagatcttcctgcggggctccacagccttccctccctcaagaggttggagatccggagttgcaggagcatcgcgaggctgccggagaagggcctcccaccttcgttcgaagaactggatatcatcgcttgcagtaatgagctagctcagcagtgcagaactctagcaagcactctgaaggtcaaaattaatgggggatatgtgaactga

SEQ ID NO: 4 - protein, 1709 aa, Oryza sativa
MEEVEAGWLEGGIRWLAETILDNLDADKLDEWIRQIRLAADTEKLRAEIEKVDGVVAAVKGRAIGNRSLARSLGRLRGLLYDADDAVDELDYFRLQQQVEGGVTTRFEAEETVGDGAEDEDDIPMDNTDVPEAVAAGSSKKRSKAWEHFTTVEFTADGKDSKARCKYCHKDLCCTSKNGTSALRNHLNVCKRKRVTSTDQPVNPSSAGEGASNATGNSVGRKRMRMDGTSTHHEAVSTHPWNKAELSNRIQCMTHQLEEAVNEVMRLCRSSSSNQSRQGTPPATNATTSSYLPEPIVYGRAAEMETIKQLIMSNRSNGITVLPIVGNGGIGKTTLAQLVCKDLVIKSQFNVKIWVYVSDKFDVVKITRQILDHVSNQSHEGISNLDTLQQDLEEQMKSKKFLIVLDDVWEIRTDDWKKLLAPLRPNDQVNSSQEEATGNMIILTTRIQSIAKSLGTVQSIKLEALKDDDIWSLFKVHAFGNDKHDSSPGLQVLGKQIASELKGNPLAAKTVGSLLGTNLTIDHWDSIIKSEEWKSLQQAYGIMQALKLSYDHLSNPLQQCVSYCSLFPKGYSFSKAQLIQIWIAQGFVEESSEKLEQKGWKYLAELVNSGFLQQVESTRFSSEYFVMHDLMHDLAQKVSQTEYATIDGSECTELAPSIRHLSIVTDSAYRKEKYRNISRNEVFEKRLMKVKSRSKLRSLVLIGQYDSHFFKYFKDAFKEAQHLRLLQITATYADSDSFLSSLVNSTHLRYLKIVTEESGRTLPRSLRKYYHLQVLDIGYRFGIPRISNDINNLLSLRHLVAYDEVCSSIANIGKMTSLQELGNFIVQNNLSGFEVTQLKSMNKLVQLSVSQLENVRTQEEACGAKLKDKQHLEKLHLSWKDAWNGYDSDESYEDEYGSDMNIETEGEELSVGDANGAQSLQHHSNISSELASSEVLEGLEPHHGLKYLRISGYNGSTSPTWLPSSLTCLQTLHLEKCGKWQILPLERLGLLVKLVLIKMRNATELSIPSLEELVLIALPSLNTCSCTSIRNLNSSLKVLKIKNCPVLKVFPLFEISQKFEIERTSSWLPHLSKLTIYNCPLSCVHSSLPPSAISGYGEYGRCTLPQSLEELYIHEYSQETLQPCFSGNLTLLRKLHVLGNSNLVSLQLHSCTALEELIIQSCESLSSLDGLQLLGNLRLLQAHRCLSGHGEDGRCILPQSLEELYIHEYSQETLQPCFSGNLTLLRKLHVMGNSNLVSLQLHSCTALEELIIQSCESLSSLDGLQLLGNLRLLRAHRCLSGHGEDGRCILPQSLEELYIHEYSQETLQPCFSGNLTLLRKLHVLENSNLVSLQLHSCTALEELIIQSCESLSSLDGLQLLGNLRLLQAHRCLSGHGEDGRCILPQSLEELYIHEYSQETLQPCFSGNLTLLRKLHVLGNSNLVSLQLHSCTSLEELKIQSCESLSSLDGLQLLGNLRLLQAHRCLSGHGEDGRCILPQSLEELFISEYSLETLQPCFLTNLTCLKQLEVSGTTSLKSLELQSCTALEHLKIQGCASLATLEGLQFLHALRHMKVFRCPGLPPYLGSSSEQGYELCPRLERLDIDDPSILTTSFCKHLTSLQRLELNYCGSEVARLTDEQERALQLLTSLQELRFKYCYNLIDLPAGLHSLPSLKRLEIRSCRSIARLPEKGLPPSFEELDIIACSNELAQQCRTLASTLKVKINGGYVN

SEQ ID NO: 5 - DNA, 3015 bp, Xanthomonas oryzae
atggatcccattcgttcgcgcacgccaagtcctgcccgcgagcctctgcccggaccccaaccggatagggttcagccgactgcagatcgtggggtgtctgcgcctgctggcagccctctggatggcttgcccgctcggcggacggtgtcccggacccggctgccatctccccctgcccccttgcctgcgttctcggcgggcagctccaccgatcggctccgtccgttcgatccgtcgcttcctgatacatcgctttttgattcgatgcctgccgtcggcacgcctcatacagaggctgccccagcagacacttcgccggccgcgcaggtggatctactcacgctcgcgacagtggcgcagcaccacgaggcactggtgggccatgggtttacacacgcgcacatcgttgcgctcagccaacacccggcagcgttagggaccgttgctgtcacgtatcaagacataatcacggcgttgccagaggcgacacacgaagacatcgttggcgtcggcaaacagttgtccggcgcacgcgccctggaggccttgctcacgaaggcgggggagttgagaggtccgccgttacagttggacacaggccaacttctcaagattgcaagacgtggcggcgtgaccgcagtggaggcagtgcatgcatggcgcaatgcactgacgggtgcccccctgaacctgaccccggaccaagtggtggccatcgccagcaatagtggcggcaagcaggcgctggagacggtgcagcggctgttgccggtgctgtgccaggaccatggcctgaccccggaccaggtcgtggccatcgccagccacgatggcggcaagcaggcgctggagacggtgcagcggctgttgccggtgctgtgccaggaccatggcctgaccccggaccaggtggtggccatcgccagcaatggcggcggcaagcaggcgctggagacggtactgtgtcaggcccatggcctgaccccggcgcaggtggtggccatcgccagcaatggcggcggcaagcaggcgctggagacggtgcagcggctgttgccggtgctgtgccaggcccatggcctgaccctggaccaggtcgtggccattgccagcaatggcggcggcaagcaggcgctggagacggtgcagcggctgttgccggtgctgtgccaggcccatggcctgaccccggaccaggtggtggccatcgccagccacgatggcggcaagcaggcgctggagacggtgcagcggctgttgccggtgctgtgccaggcccatggcctgaccccggcccaggtggtggccatcgccagccacgatggcggcaagcaggcgctggagacggtgcagcggctgttgccggtgctgtgccaggcccatggtctgaccctggaccaggtcgtggccattgccagcaatggcggcggcaagcaggcgctggagacggtgcagcggctgttgccggtgctgtgccaggcccatggtctgaccccggaccaggtcgtggccatcgccagccacgatggcggcaagcaggcgctggagacggtgcagcggctgttgccggtgctgtgccaggaccatggtctgaccccggcgcaggtggtggccatcgccagcaataacggcggcaagcaggcgctggagacggtgcagcggctgttgccggtgctgtgccaggcccatggtctgaccctggaccaggtcgtggccattgccagcaatggcggcggcaagcaggcgctggagacggtgcagcggctgttgccggtgctgtgccaggcccatggtctgaccccggaccaggtcgtggccatcgccagccacgatggcggcaagcaggcgctggagacggtgcagcggctgttgccggtgctgtgccaggaccatggtctgaccccggcgcaggtggtggccatcgccagcaataacggcggcaagcaggcgctggagacggtgcagcggctgttgccggtgctgtgccaggcccatggcctgaccccggaccaggtggtggccatcgccagccacgatggcggcaagcaggcgctggagacggtgcagcggctgttgccggtgctgtgccaggcccatggcctgaccccggaccaggtcgtggccattgccagcaatggcggcggcaagcaggcgctggagacggtgcagcggctgttgccggtgctgtgccaggcccatggtctgaccccggaccaggtcgtggccatcgccagccacgatggcggcaagcaggcgctggagacggtgcagcggctgttgccggtgctgtgccaggaccatggcctgaccccggaccaggtcgtggccatcgccagcaatattggcggcaagcaggcgctggagacggtgcagcggctgttgccggtgctgtgccaggaccatggcctgaccccggaccaggtcgtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagagtcaatagccgtattggcgaacgcacgtcccatcgcgttgccgacctcgcgcacgtggtgcgcgtgcttggttttttccagagccactcccacccagcgcaagcattcgatgacgccatgacgcagttcgggatgagcaggcacgggttggtacagctctttcgcagagtgggcgtcaccgaattcgaagcccgctgcggaactatccccccagcctcgcagcgttgggaccgtatcctccaggcatcagggacgaaaagggccaaaccgtcccctacttcagctcagacgccggatcaggcgtctttgcatgcattccccgactcgctggagcgtgaccttgatgcgcccagcccaatgcacgagggagatcagacgcgggcaagcagacgtaaacggtcctga

SEQ ID NO: 6 - protein, 1004 aa, Xanthomonas oryzae
MDPIRSRTPSPAREPLPGPQPDRVQPTADRGVSAPAGSPLDGLPARRTVSRTRLPSPPAPLPAFSAGSSTDRLRPFDPSLPDTSLFDSMPAVGTPHTEAAPADTSPAAQVDLLTLATVAQHHEALVGHGFTHAHIVALSQHPAALGTVAVTYQDIITALPEATHEDIVGVGKQLSGARALEALLTKAGELRGPPLQLDTGQLLKIARRGGVTAVEAVHAWRNALTGAPLNLTPDQVVAIASNSGGKQALETVQRLLPVLCQDHGLTPDQVVAIASHDGGKQALETVQRLLPVLCQDHGLTPDQVVAIASNGGGKQALETVLCQAHGLTPAQVVAIASNGGGKQALETVQRLLPVLCQAHGLTLDQVVAIASNGGGKQALETVQRLLPVLCQAHGLTPDQVVAIASHDGGKQALETVQRLLPVLCQAHGLTPAQVVAIASHDGGKQALETVQRLLPVLCQAHGLTLDQVVAIASNGGGKQALETVQRLLPVLCQAHGLTPDQVVAIASHDGGKQALETVQRLLPVLCQDHGLTPAQVVAIASNNGGKQALETVQRLLPVLCQAHGLTLDQVVAIASNGGGKQALETVQRLLPVLCQAHGLTPDQVVAIASHDGGKQALETVQRLLPVLCQDHGLTPAQVVAIASNNGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASHDGGKQALETVQRLLPVLCQDHGLTPDQVVAIASNIGGKQALETVQRLLPVLCQDHGLTPDQVVAIASNGGKQALESIVAQLSRPDPALAALTNDHLVALACLGGRPALDAVKKGLPHAPELIRRVNSRIGERTSHRVADLAHVVRVLGFFQSHSHPAQAFDDAMTQFGMSRHGLVQLFRRVGVTEFEARCGTIPPASQRWDRILQASGTKRAKPSPTSAQTPDQASLHAFPDSLERDLDAPSPMHEGDQTRASRRKRS

SEQ ID NO: 7 - DNA, 2592 bp, Xanthomonas oryzae
atggatcccattcgttcgcgcacgccaagtcctgcccgcgagcctctgcccggaccccaaccggatagggttcagccgactgcagatcgtggggtgtctgcgcctgctggcagccctctggatggcttgcccgctcggcggacggtgtcccggacccggctgccatctccccctgcccccttgcctgcgttctcggcgggcagctccaccgatcggctccgtccgttcgatccgtcgcttcctgatacatcgctttttgattcgatgcctgccgtcggcacgcctcatacagaggctgccccagcagacacttcgccggccgcgcaggtggatctactcacgctcgcgacagtggcgcagcaccacgaggcactggtgggccatgggtttacacacgcgcacatcgttgcgctcagccaacacccggcagcgttagggaccgttgctgtcacgtatcaagacataatcacggcgttgccagaggcgacacacgaagacatcgttggcgtcggcaaacagttgtccggcgcacgcgccctggaggccttgctcacgaaggcgggggagttgagaggtccgccgttacagttggacacaggccaacttctcaagattgcaagacgtggcggcgtgaccgcagtggaggcagtgcatgcatggcgcaatgcactgacgggtgcccccctgaacctgaccccggaccaagtggtggccatcgccagcaatagtggcggcaagcaggcgctggagacggtgcagcggctgttgccggtgctgtgccaggaccatggcctgaccccggaccaggtcgtggccatcgccagccacgatggcggcaagcaggcgctggagacggtgcagcggctgttgccggtgctgtgccaggaccatggcctgaccccggaccaggtggtggccatcgccagcaatggcggcggcaagcaggcgctggagacggtgcagcggctgttgccggtgctgtgccaggaccatggcctgaccccggaccaggtggtggccatcgccagcaatggcggcggcaagcaggcgctggagacggtactgtgccaggcccatggcctgaccccggcgcaggtggtggccatcgccagcaatggcggcggcaagcaggcgctggagacggtgcagcggctgttgccggtgctgtgccaggcccatggcctgaccctggaccaggtcgtggccattgccagcaatggcggcggcaagcaggcgctggagacggtgcagcggctgttgccggtgctgtgccaggcccatggcctgaccccggcgcaggtggtggccatcgccagcaatggcggcggcaagcaggcgctggagacggtgcagcggctgttgccggtgctgtgccaggcccatggcctgaccccggcccaggtggtggccatcgccagccacgatggcggcaagcaggcgctggagacggtgcagcggctgttgccggtgctgtgccaggcccatggtctgaccctggaccaggtagtggccattgccagccacgatggcggcaagcaggcgctggagacggtgcagcggctgttgccggtgctgtgccaggcccatggtctgaccctggaccaggtcgtggccatcgccagccacgatggcggcaagcaggcgctggagacggtgcagcggctgttgccggtgctgtgccaggaccatggtctgaccccggcgcaggtggtggccatcgccagcaataacggcggcaagcaggcgctggagacggtgcagcggctgttgccggtgctgtgccaggaccatggcctgaccccggaccaggtggtggccatcgccagccacgatggcggcaagcaggcgctggagacgatgcagcggctgttgccggtgctgtgccaggcccatggcctgaccccggaccaggtcgtggccatcgccagcaatggcggcggcaagcaggcgctggagacggtgcagcggctgttgccggtgctgtgccaggcccatggcctgaccccggaccaggtcgtggccatcgccagccacgatggcggcaagcaggcgctggagacggtgcagcggctgttgccggtgctgtgccaggaccatggcctgaccccggaccaggtcgtggccatcgccagccacgatggcggcaagcaggcgctggagacggtgcaacggctgttgcaggtgctgtgccaggaccatggcctgaccccggaccaggtcgtggccatcgccagccacgatggcggcaagcaggcgctggagacggtgcaacggctgttgccggtgctgtgccaggaccatggcctgaccccggaccaggtcgtggccatcgccagccacgatggcggcaagcaggcgctggagacggtgcagcggctgttgccggtgctgtgccaggaccatggcctgaccccggaccaggtcgtggccatcgccagcaatggcggcaagcaggcgctggagagcattgttgcccagttatctcgccgtgatccggcgttggccgcgttgaccaacgaccaactcgtcgccttggcctgcctcggcggacgtcctgccccgcattcaaggaagaggaaatcgcatgattga

SEQ ID NO: 8 - protein, 863 aa, Xanthomonas oryzae
MDPIRSRTPSPAREPLPGPQPDRVQPTADRGVSAPAGSPLDGLPARRTVSRTRLPSPPAPLPAFSAGSSTDRLRPFDPSLPDTSLFDSMPAVGTPHTEAAPADTSPAAQVDLLTLATVAQHHEALVGHGFTHAHIVALSQHPAALGTVAVTYQDIITALPEATHEDIVGVGKQLSGARALEALLTKAGELRGPPLQLDTGQLLKIARRGGVTAVEAVHAWRNALTGAPLNLTPDQVVAIASNSGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVLCQAHGLTPAQVVAIASNGGGKQALETVQRLLPVLCQAHGLTLDQVVAIASNGGGKQALETVQRLLPVLCQAHGLTPAQVVAIASNGGGKQALETVQRLLPVLCQAHGLTPAQVVAIASHDGGKQALETVQRLLPVLCQAHGLTLDQVVAIASHDGGKQALETVQRLLPVLCQAHGLTLDQVVAIASHDGGKQALETVQRLLPVLCQDHGLTPAQVVAIASNNGGKQALETVQRLLPVLCQDHGLTPDQVVAIASHDGGKQALETMQRLLPVLCQAHGLTPDQVVAIASNGGGKQALETVQRLLPVLCQAHGLTPDQVVAIASHDGGKQALETVQRLLPVLCQDHGLTPDQVVAIASHDGGKQALETVQRLLQVLCQDHGLTPDQVVAIASHDGGKQALETVQRLLPVLCQDHGLTPDQVVAIASHDGGKQALETVQRLLPVLCQDHGLTPDQVVAIASNGGKQALESIVAQLSRRDPALAALTNDQLVALACLGGRPAPHSRKRKSHD

SEQ ID NO: 9 - DNA, 27 bp, Xanthomonas oryzae
aaacggtcccgatcggatcgtgctgtc

SEQ ID NO: 10 - protein, 9 aa, Xanthomonas oryzae
KRSRSDRAV

SEQ ID NO: 11 - DNA, 27 bp, Xanthomonas oryzae
aaacggtcctgatcggatcgtgctgtc

SEQ ID NO: 12 - DNA, 15 bp, Xanthomonas oryzae
ggacgtcctgccccg

SEQ ID NO: 13 - protein, 5 aa, Xanthomonas oryzae
GRPAP

SEQ ID NO: 14 - DNA, 30 bp, Xanthomonas oryzae
cattcaacgaagaggagctcgcatggttga

SEQ ID NO: 15 - protein, 10 aa, Xanthomonas oryzae
AFNEEELAWL

SEQ ID NO: 16 - DNA, 45 bp, Xanthomonas oryzae
ggacgtcctgccccgcattcaaggaagaggaaatcgcatgattga

SEQ ID NO: 17 - protein, 14 aa, Xanthomonas oryzae
GRPAPHSRKRKSHD

SEQ ID NO: 18 - protein, 286 aa, Xanthomonas oryzae
SIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTGASSRKRSRSDRAVTGPSAQHSFEVRVPEQRDALHLPLSWRVKRPRTRIGGGLPDPGTPIAADLAASSTVMWEQDAAPFAGAADDFPAFNEEELAWLMELLPQSGSVGGTI

SEQ ID NO: 19 - protein, 183 aa, Xanthomonas oryzae
SIVAQLSRPDPALAALTNDHLVALACLGGRPALDAVKKGLPHAPELIRRVNSRIGERTSHRVADLAHVVRVLGFFQSHSHPAQAFDDAMTQFGMSRHGLVQLFRRVGVTEFEARCGTIPPASQRWDRILQASGTKRAKPSPTSAQTPDQASLHAFPDSLERDLDAPSPMHEGDQTRASRRKRS

SEQ ID NO: 20 - protein, 42 aa, Xanthomonas oryzae
SIVAQLSRRDPALAALTNDQLVALACLGGRPAPHSRKRKSHD

SEQ ID NO: 21 - protein, 7 aa, Xanthomonas oryzae
TKRAKPS

SEQ ID NO: 22 - protein, 6 aa, Xanthomonas oryzae
SRRKRS

SEQ ID NO: 23 - protein, 6 aa, Xanthomonas oryzae
SRKRKS

SEQ ID NO: 24 - protein, 7 aa, Artificial Sequence
  Mutated sequence
TAAAAPS

SEQ ID NO: 25 - protein, 6 aa, Artificial Sequence
  Mutated sequence
SAAAAS

SEQ ID NO: 26 - protein, 14 aa, Artificial Sequence
  Mutated sequence
SAAAASRPKKKRKV

SEQ ID NO: 27 - protein, 7 aa, Xanthomonas oryzae
MKRAKPS

SEQ ID NO: 28 - protein, 7 aa, Xanthomonas oryzae
SRKRSRS

SEQ ID NO: 29 - protein, 8 aa, Xanthomonas oryzae
WRVKRPRT

SEQ ID NO: 30 - protein, 7 aa, Artificial Sequence
  Mutated sequence
MAAAAPS

SEQ ID NO: 31 - protein, 7 aa, Artificial Sequence
  Mutated sequence
SAAAAAS

SEQ ID NO: 32 - protein, 8 aa, Artificial Sequence
  Mutated sequence
WAAAAAAT

SEQ ID NO: 33 - protein, 8 aa, Simian virus 40
RPKKKRKV

SEQ ID NO: 34 - DNA, 32 bp, Artificial Sequence
  Primer
atatatctagacggcagtgatggcgaacggtt

SEQ ID NO: 35 - DNA, 32 bp, Artificial Sequence
  Primer
atatagagctctcgttggccaggcgcagctcg

SEQ ID NO: 36 - DNA, 32 bp, Artificial Sequence
  Primer
atatagagctcgccggcgacatcgcccaccgc

SEQ ID NO: 37 - DNA, 32 bp, Artificial Sequence
  Primer
tatatgtcgacaaaggtccgtgcggcatctgg

SEQ ID NO: 38 - DNA, 27 bp, Artificial Sequence
  Primer
tatatcccgggatgctggcggccagta

SEQ ID NO: 39 - DNA, 27 bp, Artificial Sequence
  Primer
tatataagcttcatgcattcgccgatt

SEQ ID NO: 40 - DNA, 27 bp, Artificial Sequence
  Primer
tatataagcttcactgcctccactgcg

SEQ ID NO: 41 - DNA, 27 bp, Artificial Sequence
  Primer
tatatgtcgacccacatctgcggcgca

SEQ ID NO: 42 - DNA, 28 bp, Artificial Sequence
  Primer
atacccgggcatggcggaatccggtgcg

SEQ ID NO: 43 - DNA, 29 bp, Artificial Sequence
  Primer
aatactagtaatcttgagaagttggcctg

SEQ ID NO: 44 - DNA, 29 bp, Artificial Sequence
  Primer
ataactagttcgcatgattgatggagcta

SEQ ID NO: 45 - DNA, 29 bp, Artificial Sequence
  Primer
ttagcatgctcgtacgcatgaaggctgga

SEQ ID NO: 46 - DNA, 35 bp, Artificial Sequence
  Primer
atatacccgggatgcattttttggcgaagggcact

SEQ ID NO: 47 - DNA, 35 bp, Artificial Sequence
  Primer
atatatctagaacatccgctggttgctgcgggcca

SEQ ID NO: 48 - DNA, 35 bp, Artificial Sequence
  Primer
atatatctagagtggacctgctcaagcgaatgatg

SEQ ID NO: 49 - DNA, 35 bp, Artificial Sequence
  Primer
tatatgtcgacttctggcgcaacttcggccaggca

SEQ ID NO: 50 - DNA, 33 bp, Artificial Sequence
  Primer
atatacccgggagcaatggccgcatgagccagg

SEQ ID NO: 51 - DNA, 33 bp, Artificial Sequence
  Primer
tatatgcatgcgccgccgcaagcgccgtcggcg

SEQ ID NO: 52 - DNA, 33 bp, Artificial Sequence
  Primer
tgcgccatgcatgcactgcctccactgcggtca

SEQ ID NO: 53 - DNA, 33 bp, Artificial Sequence
  Primer
tatatgtcgaccacaatcaatggcctgctgggc

SEQ ID NO: 54 - DNA, 35 bp, Artificial Sequence
  Primer
atatacccgggatggcaatgagatatggttgaacc

SEQ ID NO: 55 - DNA, 35 bp, Artificial Sequence
  Primer
tatatggatcctcaccgctgaaagtgcgtgctaat

SEQ ID NO: 56 - DNA, 35 bp, Artificial Sequence
  Primer
atataggatccgatcctggtacgcccatcgctgcc

SEQ ID NO: 57 - DNA, 34 bp, Artificial Sequence
  Primer
tatatgtcgaccgcagcaagcagcgcttgtggac

SEQ ID NO: 58 - DNA, 30 bp, Artificial Sequence
  Primer
ggacccggggtagggaccacagaccgctag

SEQ ID NO: 59 - DNA, 30 bp, Artificial Sequence
  Primer
ccaaagcttactgtcgaacgcaccttcggt

SEQ ID NO: 60 - DNA, 28 bp, Artificial Sequence
  Primer
tggaagcttgaccttgatgcgcctagcc

SEQ ID NO: 61 - DNA, 28 bp, Artificial Sequence
  Primer
tcctctagactgaggcaatagctccatc

SEQ ID NO: 62 - DNA, 35 bp, Artificial Sequence
  Primer
atatacccgggatgctcaagaacgatcgcctgctg

SEQ ID NO: 63 - DNA, 35 bp, Artificial Sequence
  Primer
tatatgcatgcacccgaatcctgggtgacacgggc

SEQ ID NO: 64 - DNA, 35 bp, Artificial Sequence
  Primer
atatagcatgcatttttcaccacttctgagaagcg

SEQ ID NO: 65 - DNA, 35 bp, Artificial Sequence
  Primer
tatatgtcgacccttgccgagagttcaagacctgg

SEQ ID NO: 66 - DNA, 31 bp, Artificial Sequence
  Primer
cgttggccgcgttgaccaacgaccacctcgt

SEQ ID NO: 67 - DNA, 55 bp, Artificial Sequence
  Primer
tataagcttcacttatcgtcatcgtccttgtaatcggaccgtttacgtctgcttg

SEQ ID NO: 68 - DNA, 31 bp, Artificial Sequence
  Primer
cgttggccgcgttgaccaacgaccaactcgt

SEQ ID NO: 69 - DNA, 61 bp, Artificial Sequence
  Primer
tataagcttcacttatcgtcatcgtccttgtaatcatcatgcgatttcctcttccttgaat

SEQ ID NO: 70 - DNA, 23 bp, Artificial Sequence
  Primer
cgacccggggcacccgtgtcacg

SEQ ID NO: 71 - DNA, 33 bp, Artificial Sequence
  Primer
atggatcctggcgcatcgccatcgccgctatgg

SEQ ID NO: 72 - DNA, 31 bp, Artificial Sequence
  Primer
tgggatccatcaggcatacctctttggagaa

SEQ ID NO: 73 - DNA, 31 bp, Artificial Sequence
  Primer
atgtcgactcatgctgcacaccaagccgtgg

SEQ ID NO: 74 - DNA, 39 bp, Artificial Sequence
  Primer
cggaggggttggatcctacgacacgcatcggtagatctg

SEQ ID NO: 75 - DNA, 37 bp, Artificial Sequence
  Primer
cgagggcccgggatccgtcgctcagatagtcccccga

SEQ ID NO: 76 - DNA, 17 bp, Artificial Sequence
  Primer
cagacgtaaacggtcct

SEQ ID NO: 77 - DNA, 20 bp, Artificial Sequence
  Primer
acgctgccaggtcggcaacc

SEQ ID NO: 78 - DNA, 18 bp, Artificial Sequence
  Primer
gacgtcctgccccgcatt

SEQ ID NO: 79 - DNA, 19 bp, Artificial Sequence
  Primer
ggacgtcgctcagatagtc

SEQ ID NO: 80 - DNA, 21 bp, Artificial Sequence
  Primer
tggtagtccacgccctaaacg

SEQ ID NO: 81 - DNA, 21 bp, Artificial Sequence
  Primer
ctggaaagttccgtggatgtc

SEQ ID NO: 82 - DNA, 41 bp, Artificial Sequence
  Primer
tgattacgaattcgagctaacaacttttctttttctgaatc

SEQ ID NO: 83 - DNA, 32 bp, Artificial Sequence
  Primer
tcattaccaaaagcatgcactttaaatagtga

SEQ ID NO: 84 - DNA, 32 bp, Artificial Sequence
  Primer
tggtcactatttaaagtgcatgcttttggtaa

SEQ ID NO: 85 - DNA, 42 bp, Artificial Sequence
  Primer
tagaggatccccgggtaccgtgacaatgcattggagcggatt

SEQ ID NO: 86 - DNA, 21 bp, Artificial Sequence
  Primer
aactgattactcggtggcttg

SEQ ID NO: 87 - DNA, 18 bp, Artificial Sequence
  Primer
tgtaaaacgacggccagt

SEQ ID NO: 88 - DNA, 18 bp, Artificial Sequence
  Primer
cgtcttcatcacctgcaa

SEQ ID NO: 89 - DNA, 19 bp, Artificial Sequence
  Primer
tcagcgtacgatagtagta

SEQ ID NO: 90 - DNA, 18 bp, Artificial Sequence
  Primer
ctcaagatgatcgaggac

SEQ ID NO: 91 - DNA, 18 bp, Artificial Sequence
  Primer
cgtcttcatcacctgcaa

SEQ ID NO: 92 - DNA, 18 bp, Artificial Sequence
  Primer
acgacataaacgggccac

SEQ ID NO: 93 - DNA, 19 bp, Artificial Sequence
  Primer
aggtgctaatgccatggct

SEQ ID NO: 94 - DNA, 20 bp, Artificial Sequence
  Primer
ctcagcacattccagcagat

SEQ ID NO: 95 - DNA, 20 bp, Artificial Sequence
  Primer
acagataggccggttgaaaa